(19) United States
(12) Patent Application Publication
(10) Pub. No.: US 2025/0201339 A1
(43) Pub. Date: Jun. 19, 2025

(54) USE OF COMBINED CD274 COPY NUMBER CHANGES AND TMB TO PREDICT RESPONSE TO IMMUNOTHERAPIES

(71) Applicant: Foundation Medicine, Inc., Boston, MA (US)

(72) Inventors: Richard Sheng Poe HUANG, Cary, NC (US); Karthikeyan MURUGESAN, Boston, MA (US)

(73) Assignee: Foundation Medicine, Inc., Boston, MA (US)

(21) Appl. No.: 18/845,602

(22) PCT Filed: Mar. 16, 2023
(86) PCT No.: PCT/US2023/064602

§ 371 Date: Sep. 10, 2024

Related U.S. Application Data
(60) Provisional application No. 63/321,612, filed on Mar. 18, 2022.

(51) Int. Cl. G16B 20/10; C12Q 1/6855; C12Q 1/6869; G01N 33/68; G16B 20/20; G16H 15/00; G16H 20/17

(52) U.S. Cl. CPC G16B 20/10; C12Q 1/6855; C12Q 1/6869; G01N 33/6893; G16B 20/20; G16H 15/00; G16H 20/17; C12Q 2600/106; G01N 2333/70532

(57) ABSTRACT

Provided herein are methods related to detecting cluster of differentiation 274 (CD274) copy number alterations and/or tumor mutational burden (TMB), as well as methods of treatment and uses related thereto. Detection of CD274 copy number alterations and/or TMB can be used to identify individuals having cancer that may benefit from treatment with an immunotherapy.

### CROSS-REFERENCE TO RELATED APPLICATIONS

This application claims the benefit of U.S. Provisional Application No. 63/321,612, filed Mar. 18, 2022, which is hereby incorporated by reference in its entirety.

### REFERENCE TO AN ELECTRONIC SEQUENCE LISTING

The contents of the electronic sequence listing (197102008740seqlist.xml; Size: 6,705 bytes; and Date of Creation: Mar. 7, 2023) are herein incorporated by reference in their entirety.

### TECHNICAL FIELD

Provided herein are methods related to detecting cluster of differentiation 274 (CD274) copy number alterations and/or tumor mutational burden (TMB) in cancer, as well as methods of diagnosis/treatment and uses related thereto.

### BACKGROUND

Immunotherapies, such as immune checkpoint inhibitors (ICIs) have been approved for use in multiple tumor types, and are incorporated into the National Comprehensive Cancer Network (NCCN) guidelines, influencing real world clinical management of patients with cancer (Vaddepally et al., Cancers (2020) 12, 738). Despite this, only an estimated 12.5% of eligible patients (based on PD-L1 positivity) are reported to respond to ICI treatment (Haslam et al., JAMA Netw Open 2 (2019) e192535).

PD-L1 expression as detected by immunohistochemistry (IHC) has identified a subset of tumors more responsive to ICI treatment (Topalian et al., N. Engl. J. Med. 2012), and is a US Food and Drug Administration (FDA) approved companion diagnostic (CDx) in multiple tumor types (U.S. FDA, List of Cleared or Approved Companion Diagnostic Devices [In Vitro and Imaging Tools]; available at the website: www.fda.gov/medical-devices/vitro-diagnostics/list-cleared-or-approved-companion-diagnostic-devices-vitro-and-imaging-tools). However, PD-L1 IHC testing is complex and remains insufficient to consistently predict responses to ICI treatment (Jardim et al., Cancer Cell (2021) 39, 154-173; Remon et al., BMC Medicine (2017); Garon, N. Engl. J. Med. (2017); Huang et al., Mod. Pathol. (2020)).

Other biomarkers, such as high tumor mutational burden (TMB-High) and microsatellite instability-high (MSI-H) have also been used to select patients for treatment with ICIs. In particular, TMB-High (e.g., with a TMB greater than or equal to 10 mutations/Megabase [mut/Mb]) and MSI-H solid tumor patients are eligible to receive ICI treatment based on two pan-solid tumor approvals (Subbiah et al., Ann. Oncol. (2020) 31, 1115-1118; Marcus et al., Clin. Cancer Res (2019) 25, 3753-3758). However, the clinical outcomes of ICI treatment in these biomarker positive patients is varied (Huang et al., Mod. Pathol. (2020); Strickler et al., Clin. Cancer Res. (2021) 27, 1236 LP-1241).

Thus, there is a need in the art for improved predictive biomarkers of response to immunotherapies, such as ICIs, in cancer to guide the treatment of cancer patients.

All references cited herein, including patents, patent applications and publications, are hereby incorporated by reference in their entirety. To the extent that any reference incorporated by reference conflicts with the instant disclosure, the instant disclosure shall control.

### SUMMARY OF THE INVENTION

In one aspect, provided herein is a method of identifying an individual having a cancer who may benefit from a treatment comprising an anti-cancer therapy, the method comprising detecting in one or more samples from the individual a cluster of differentiation 274 (CD274) gene copy number gain and a high tumor mutational burden (TMB), wherein detection of the CD274 gene copy number gain and the high TMB in the one or more samples identifies the individual as one who may benefit from a treatment comprising an anti-cancer therapy.

In another aspect, provided herein is a method of selecting a treatment for an individual having a cancer, the method comprising detecting in one or more samples from the individual a CD274 gene copy number gain and a high TMB, wherein detection of the CD274 gene copy number gain and the high TMB in the one or more samples identifies the individual as one who may benefit from a treatment comprising an anti-cancer therapy.

In another aspect, provided herein is a method of identifying one or more treatment options for an individual having a cancer, the method comprising: (a) detecting in one or more samples from the individual a CD274 gene copy number gain and a high TMB; and (b) generating a report comprising one or more treatment options identified for the individual based, at least in part, on detection of the CD274 gene copy number gain and the high TMB in the one or more samples, wherein the one or more treatment options comprise an anti-cancer therapy.

In another aspect, provided herein is a method of identifying one or more treatment options for an individual having a cancer, the method comprising: (a) acquiring knowledge of a CD274 gene copy number gain and a high TMB in one or more samples from the individual; and (b) generating a report comprising one or more treatment options identified for the individual based, at least in part, on said knowledge, wherein the one or more treatment options comprise an anti-cancer therapy.

In some embodiments, the report further indicates the presence or absence of the CD274 gene copy number gain and the high TMB in the one or more samples.

In another aspect, provided herein is a method of selecting a treatment for an individual having a cancer, comprising acquiring knowledge of a CD274 gene copy number gain and a high TMB in one or more samples from the individual, wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

In another aspect, provided herein is a method of predicting survival of an individual having a cancer, comprising acquiring knowledge of a CD274 gene copy number gain and a high TMB in one or more samples from the individual, wherein responsive to the acquisition of said knowledge, the individual is predicted to have longer survival when treated with a treatment comprising an anti-cancer therapy, as compared to survival of an individual whose cancer does not comprise a CD274 gene copy number gain and/or a high TMB.

In another aspect, provided herein is a method of predicting survival of an individual having a cancer treated with a treatment comprising an anti-cancer therapy, the method comprising acquiring knowledge of a CD274 gene copy number gain and a high TMB in one or more samples from the individual, wherein responsive to the acquisition of said knowledge, the individual is predicted to have longer survival when treated with a treatment comprising an anti-cancer therapy, as compared to an individual whose cancer does not exhibit a CD274 gene copy number gain and/or a high TMB.

In another aspect, provided herein is a method of treating or delaying progression of a cancer in an individual, comprising: (a) acquiring knowledge of a CD274 gene copy number gain and a high TMB in one or more samples from an individual having a cancer; and (b) responsive to said knowledge, administering to the individual an effective amount of a treatment that comprises an anti-cancer therapy.

In another aspect, provided herein is a method of treating or delaying progression of a cancer in an individual, comprising administering to an individual having a cancer an effective amount of a treatment that comprises an anti-cancer therapy, wherein the anti-cancer therapy is administered responsive to acquiring knowledge of a CD274 gene copy number gain and a high TMB in one or more samples from the individual.

In another aspect, provided herein is a method of monitoring, evaluating or screening an individual having a cancer, comprising acquiring knowledge of a CD274 gene copy number gain and a high TMB in one or more samples from the individual, wherein responsive to the acquisition of said knowledge, the individual is predicted to have an improved response to a treatment comprising an anti-cancer therapy and/or longer survival when treated with a treatment comprising an anti-cancer therapy, as compared to an individual whose cancer does not comprise a CD274 gene copy number gain and/or a high TMB.

In another aspect, provided herein is a method for monitoring progression or recurrence of a cancer in an individual, the method comprising: (a) detecting the presence or absence of a CD274 gene copy number gain and a high TMB in one or more samples obtained from the individual at a first time point; (b) detecting the presence or absence of a CD274 gene copy number gain and a high TMB in one or more samples obtained from the individual at a second time point after the first time point; and (c) providing an assessment of cancer progression or cancer recurrence in the individual based, at least in part, on the presence or absence of the CD274 gene copy number gain and the high TMB in the one or more samples obtained from the individual at the first and/or second time points.

In some embodiments, which may be combined with any of the preceding aspects or embodiments, the presence of the CD274 gene copy number gain and the high TMB in the one or more samples obtained from the individual at the first and/or second time points identifies the individual as having decreased risk of cancer progression or cancer recurrence when treated with a treatment comprising an anti-cancer therapy. In some embodiments, the method further comprises selecting a treatment, administering a treatment, adjusting a treatment, adjusting a dose of a treatment, or applying a treatment to the individual based, at least in part, on detecting the presence of the CD274 gene copy number gain and the high TMB in the one or more samples obtained from the individual at the first and/or second time points, wherein the treatment comprises an anti-cancer therapy.

In another aspect, provided herein is a method of identifying a candidate treatment for a cancer in an individual in need thereof, comprising: performing DNA sequencing on one or more samples obtained from the individual to determine a sequencing mutation profile, wherein the sequencing mutation profile identifies the presence of a CD274 gene copy number gain and a high TMB in the one or more samples; and selecting a treatment for the individual based, at least in part, on the sequencing mutation profile, wherein the treatment comprises an anti-cancer therapy.

In some embodiments, the presence of the CD274 gene copy number gain and the high TMB in the one or more samples identifies the individual as one who may benefit from a treatment comprising an anti-cancer therapy. In some embodiments, the presence of the CD274 gene copy number gain and the high TMB in the one or more samples predicts the individual to have longer survival when treated with a treatment comprising an anti-cancer therapy, as compared to survival of an individual whose cancer does not comprise a CD274 gene copy number gain and a high TMB. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next-generation sequencing (NGS).

In another aspect, provided herein is a method of treating or delaying progression of a cancer in an individual, comprising: (a) detecting in one or more samples from an individual having a cancer a CD274 gene copy number gain and a high TMB; and (b) administering to the individual an effective amount of a treatment that comprises an anti-cancer therapy.

In some embodiments, which may be combined with any of the preceding aspects or embodiments, the anti-cancer therapy is any anti-cancer therapy or agent known in the art or provided herein. In some embodiments, which may be combined with any of the preceding aspects or embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method of identifying an individual having a cancer who may benefit from a treatment comprising an immunotherapy, the method comprising detecting in one or more samples from the individual a cluster of differentiation 274 (CD274) gene copy number gain and a high tumor mutational burden (TMB), wherein detection of the CD274 gene copy number gain and the high TMB in the one or more samples identifies the individual as one who may not benefit from a treatment comprising an anti-cancer therapy and may benefit from an immunotherapy. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method of selecting a treatment for an individual having a cancer, the method comprising detecting in one or more samples from the individual a CD274 gene copy number gain and a high TMB, wherein detection of the CD274 gene copy number gain and the high TMB in the one or more samples identifies the individual as one who may not benefit from a treatment comprising an anti-cancer therapy and may benefit from an immunotherapy. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method of selecting a treatment for an individual having a cancer, comprising acquiring knowledge of a CD274 gene copy number gain and a high TMB in one or more samples from the individual, wherein responsive to the acquisition of said knowledge the individual is identified as unlikely to respond to a treatment that comprises an anti-cancer therapy, and as likely to respond to an immunotherapy. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method of predicting survival of an individual having a cancer, comprising acquiring knowledge of a CD274 gene copy number gain and a high TMB in one or more samples from the individual, wherein responsive to the acquisition of said knowledge, the individual is predicted to have shorter survival when treated with a treatment comprising an anti-cancer therapy and longer survival when treated with a treatment comprising an immunotherapy, as compared to survival of an individual whose cancer does not comprise a CD274 gene copy number gain and/or a high TMB. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method of predicting survival of an individual having a cancer treated with a treatment comprising an anti-cancer therapy, the method comprising acquiring knowledge of a CD274 gene copy number gain and a high TMB in one or more samples from the individual, wherein responsive to the acquisition of said knowledge, the individual is predicted to have shorter survival when treated with a treatment comprising an anti-cancer therapy and longer survival when treated with a treatment comprising an immunotherapy, as compared to an individual whose cancer does not exhibit a CD274 gene copy number gain and/or a high TMB. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method of monitoring, evaluating or screening an individual having a cancer, comprising acquiring knowledge of a CD274 gene copy number gain and a high TMB in one or more samples from the individual, wherein responsive to the acquisition of said knowledge, the individual is predicted to have an improved response to a treatment comprising an immunotherapy and/or longer survival when treated with a treatment comprising an immunotherapy, as compared to a corresponding individual treated with an anti-cancer therapy. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method for monitoring progression or recurrence of a cancer in an individual, the method comprising: (a) detecting the presence or absence of a CD274 gene copy number gain and a high TMB in one or more samples obtained from the individual at a first time point; (b) detecting the presence or absence of a CD274 gene copy number gain and a high TMB in one or more samples obtained from the individual at a second time point after the first time point; and (c) providing an assessment of cancer progression or cancer recurrence in the individual based, at least in part, on the presence or absence of the CD274 gene copy number gain and the high TMB in the one or more samples obtained from the individual at the first and/or second time points. In some embodiments, which may be combined with any of the preceding aspects or embodiments, the presence of the CD274 gene copy number gain and the high TMB in the one or more samples obtained from the individual at the first and/or second time points identifies the individual as having increased risk of cancer progression or cancer recurrence when treated with a treatment comprising an anti-cancer therapy, and decreased risk of cancer progression or recurrence when treated with a treatment comprising an immunotherapy. In some embodiments, the method further comprises selecting a treatment, administering a treatment, adjusting a treatment, adjusting a dose of a treatment, or applying a treatment to the individual based, at least in part, on detecting the presence of the CD274 gene copy number gain and the high TMB in the one or more samples obtained from the individual at the first and/or second time points, wherein the treatment comprises an immunotherapy. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method of identifying a candidate treatment for a cancer in an individual in need thereof, comprising: performing DNA sequencing on one or more samples obtained from the individual to determine a sequencing mutation profile, wherein the sequencing mutation profile identifies the presence of a CD274 gene copy number gain and a high TMB in the one or more samples; and selecting a treatment for the individual based, at least in part, on the sequencing mutation profile, wherein the treatment comprises an immunotherapy. In some embodiments, the presence of the CD274 gene copy number gain and the high TMB in the one or more samples identifies the individual as one who may not benefit from a treatment comprising an anti-cancer therapy and may benefit from an immunotherapy. In some embodiments, the presence of the CD274 gene copy number gain and the high TMB in the one or more samples predicts the individual to have longer survival when treated with a treatment comprising an immunotherapy, as compared to survival of a corresponding individual treated with an anti-cancer therapy. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next-generation sequencing (NGS). In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a method of identifying an individual having a cancer who may benefit from a treatment comprising an immunotherapy, the method comprising detecting in one or more samples from the individual a cluster of differentiation 274 (CD274) gene copy number gain and a high tumor mutational burden (TMB), wherein detection of the CD274 gene copy number gain and the high TMB in the one or more samples identifies the individual as one who may benefit from a treatment comprising an immunotherapy.

In another aspect, provided herein is a method of selecting a treatment for an individual having a cancer, the method comprising detecting in one or more samples from the individual a CD274 gene copy number gain and a high TMB, wherein detection of the CD274 gene copy number gain and the high TMB in the one or more samples identifies the individual as one who may benefit from a treatment comprising an immunotherapy.

In another aspect, provided herein is a method of identifying one or more treatment options for an individual having a cancer, the method comprising: (a) detecting in one or more samples from the individual a CD274 gene copy number gain and a high TMB; and (b) generating a report comprising one or more treatment options identified for the individual based, at least in part, on detection of the CD274 gene copy number gain and the high TMB in the one or more samples, wherein the one or more treatment options comprise an immunotherapy.

In another aspect, provided herein is a method of identifying one or more treatment options for an individual having a cancer, the method comprising: (a) acquiring knowledge of a CD274 gene copy number gain and a high TMB in one or more samples from the individual; and (b) generating a report comprising one or more treatment options identified for the individual based, at least in part, on said knowledge, wherein the one or more treatment options comprise an immunotherapy.

In some embodiments, the report further indicates the presence or absence of the CD274 gene copy number gain and the high TMB in the one or more samples.

In another aspect, provided herein is a method of selecting a treatment for an individual having a cancer, comprising acquiring knowledge of a CD274 gene copy number gain and a high TMB in one or more samples from the individual, wherein responsive to the acquisition of said knowledge: (i) the individual is classified as a candidate to receive a treatment comprising an immunotherapy; and/or (ii) the individual is identified as likely to respond to a treatment that comprises an immunotherapy.

In another aspect, provided herein is a method of predicting survival of an individual having a cancer, comprising acquiring knowledge of a CD274 gene copy number gain and a high TMB in one or more samples from the individual, wherein responsive to the acquisition of said knowledge, the individual is predicted to have longer survival when treated with a treatment comprising an immunotherapy, as compared to survival of an individual whose cancer does not comprise a CD274 gene copy number gain and/or a high TMB.

In another aspect, provided herein is a method of predicting survival of an individual having a cancer treated with a treatment comprising an immunotherapy, the method comprising acquiring knowledge of a CD274 gene copy number gain and a high TMB in one or more samples from the individual, wherein responsive to the acquisition of said knowledge, the individual is predicted to have longer survival when treated with a treatment comprising an immunotherapy, as compared to an individual whose cancer does not exhibit a CD274 gene copy number gain and/or a high TMB.

In another aspect, provided herein is a method of treating or delaying progression of a cancer in an individual, comprising: (a) acquiring knowledge of a CD274 gene copy number gain and a high TMB in one or more samples from an individual having a cancer; and (b) responsive to said knowledge, administering to the individual an effective amount of a treatment that comprises an immunotherapy.

In another aspect, provided herein is a method of treating or delaying progression of a cancer in an individual, comprising administering to an individual having a cancer an effective amount of a treatment that comprises an immunotherapy, wherein the immunotherapy is administered responsive to acquiring knowledge of a CD274 gene copy number gain and a high TMB in one or more samples from the individual.

In another aspect, provided herein is a method of monitoring, evaluating or screening an individual having a cancer, comprising acquiring knowledge of a CD274 gene copy number gain and a high TMB in one or more samples from the individual, wherein responsive to the acquisition of said knowledge, the individual is predicted to have an improved response to a treatment comprising an immunotherapy and/or longer survival when treated with a treatment comprising an immunotherapy, as compared to an individual whose cancer does not comprise a CD274 gene copy number gain and/or a high TMB.

In another aspect, provided herein is a method for monitoring progression or recurrence of a cancer in an individual, the method comprising: (a) detecting the presence or absence of a CD274 gene copy number gain and a high TMB in one or more samples obtained from the individual at a first time point; (b) detecting the presence or absence of a CD274 gene copy number gain and a high TMB in one or more samples obtained from the individual at a second time point after the first time point; and (c) providing an assessment of cancer progression or cancer recurrence in the individual based, at least in part, on the presence or absence of the CD274 gene copy number gain and the high TMB in the one or more samples obtained from the individual at the first and/or second time points.

In some embodiments, the presence of the CD274 gene copy number gain and the high TMB in the one or more samples obtained from the individual at the first and/or second time points identifies the individual as having decreased risk of cancer progression or cancer recurrence when treated with a treatment comprising an immunotherapy. In some embodiments, the method further comprises selecting a treatment, administering a treatment, adjusting a treatment, adjusting a dose of a treatment, or applying a treatment to the individual based, at least in part, on detecting the presence of the CD274 gene copy number gain and the high TMB in the one or more samples obtained from the individual at the first and/or second time points, wherein the treatment comprises an immunotherapy.

In another aspect, provided herein is a method of identifying a candidate treatment for a cancer in an individual in need thereof, comprising: performing DNA sequencing on one or more samples obtained from the individual to determine a sequencing mutation profile, wherein the sequencing mutation profile identifies the presence of a CD274 gene copy number gain and a high TMB in the one or more samples; and selecting a treatment for the individual based, at least in part, on the sequencing mutation profile, wherein the treatment comprises an immunotherapy.

In some embodiments, which may be combined with any of the preceding aspects or embodiments, the presence of the CD274 gene copy number gain and the high TMB in the one or more samples identifies the individual as one who may benefit from a treatment comprising an immunotherapy. In some embodiments, the presence of the CD274 gene copy number gain and the high TMB in the one or more samples predicts the individual to have longer survival when treated with a treatment comprising an immunotherapy, as compared to survival of an individual whose cancer does not comprise a CD274 gene copy number gain and a high TMB. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next-generation sequencing (NGS).

In another aspect, provided herein is a method of treating or delaying progression of a cancer in an individual, comprising: (a) detecting in one or more samples from an individual having a cancer a CD274 gene copy number gain and a high TMB; and (b) administering to the individual an effective amount of a treatment that comprises an immunotherapy.

In some embodiments, which may be combined with any of the preceding aspects or embodiments, the CD274 gene copy number gain in a sample from the individual comprises a CD274 gene copy number of any of at least +1, at least +2, at least +3, at least +4, at least +5, at least +6, at least +7, at least +8, or more, as compared to the ploidy of the sample from the individual. In some embodiments, the CD274 gene copy number gain in a sample from the individual comprises a CD274 gene copy number of any of at least +2, at least +3, at least +4, at least +5, at least +6, at least +7, at least +8, or more, as compared to the ploidy of the sample from the individual. In some embodiments, the CD274 gene copy number gain in a sample from the individual comprises a CD274 gene copy number of at least +2, as compared to the ploidy of the sample from the individual. In some embodiments, the ploidy of the sample from the individual is monoploid, diploid, triploid, tetraploid.

In some embodiments, which may be combined with any of the preceding aspects or embodiments, the CD274 gene copy number gain in a sample from the individual comprises a CD274 gene copy number of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is monoploid; the CD274 gene copy number gain in a sample from the individual comprises a CD274 gene copy number of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is diploid; the CD274 gene copy number gain in a sample from the individual comprises a CD274 gene copy number of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is triploid; or the CD274 gene copy number gain in a sample from the individual comprises a CD274 gene copy number of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is tetraploid. In some embodiments, which may be combined with any of the preceding aspects or embodiments, the CD274 gene copy number is any of at least four, at least five, at least six, or more. In some embodiments, which may be combined with any of the preceding aspects or embodiments, the CD274 gene copy number is at least four.

In some embodiments, which may be combined with any of the preceding aspects or embodiments, acquiring knowledge of a CD274 gene copy number gain and a high TMB comprises detecting the CD274 gene copy number gain and the high TMB in the one or more samples. In some embodiments, which may be combined with any of the preceding aspects or embodiments, the CD274 gene copy number gain is detected by fluorescence in situ hybridization (FISH), comprehensive genomic profiling (CGP), comparative genomic hybridization (CGH), sequencing, or any combination thereof. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next-generation sequencing (NGS).

In some embodiments, which may be combined with any of the preceding aspects or embodiments, detecting a CD274 gene copy number gain comprises: (a) providing a plurality of nucleic acid molecules obtained from a sample from an individual having a cancer, wherein the plurality of nucleic acid molecules comprises nucleic acid molecules corresponding to a CD274 gene, or a portion thereof; (b) optionally, ligating one or more adapters onto one or more nucleic acid molecules from the plurality of nucleic acid molecules; (c) optionally, amplifying the one or more ligated nucleic acid molecules from the plurality of nucleic acid molecules; (d) optionally, capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more sequence reads of the plurality of sequence reads correspond to a CD274 gene, or a portion thereof; (f) analyzing the plurality of sequence reads for the presence or absence of a CD274 gene copy number gain; and (g) based on the analyzing step, detecting the presence or absence of a CD274 gene copy number gain in the sample. In some embodiments, the sequencer comprises a next-generation sequencer.

In some embodiments, which may be combined with any of the preceding aspects or embodiments, detecting a CD274 gene copy number gain comprises: (a) providing a sample from an individual having a cancer, wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a CD274 gene or a portion thereof in said library to produce an enriched sample; (e) sequencing the enriched sample, thereby producing a plurality of sequence reads; (f) analyzing the plurality of sequence reads for the presence or absence of a CD274 gene copy number gain; and (g) detecting, based on the analyzing step, the presence or absence of a CD274 gene copy number gain in the sample from the individual.

In some embodiments, which may be combined with any of the preceding aspects or embodiments, the one or more adapters comprise amplification primers, flow cell adapter sequences, substrate adapter sequences, sample index sequences, or unique molecular identifier (UMI) sequences. In some embodiments, which may be combined with any of the preceding aspects or embodiments, the selectively enriching comprises: (a) combining one or more bait molecules with the library, thereby hybridizing the one or more bait molecules to one or more nucleic acid molecules comprising nucleotide sequences corresponding to a CD274 gene or a portion thereof, and producing nucleic acid hybrids; and (b) isolating the nucleic acid hybrids to produce the enriched sample. In some embodiments, which may be combined with any of the preceding aspects or embodiments, the amplified nucleic acid molecules are captured by hybridization with one or more bait molecules. In some embodiments, which may be combined with any of the preceding aspects or embodiments, the amplifying comprises performing a polymerase chain reaction (PCR) amplification technique, a non-PCR amplification technique, or an isothermal amplification technique. In some embodiments, which may be combined with any of the preceding aspects or embodiments, the method further comprises selectively enriching for one or more nucleic acid molecules in the sample comprising nucleotide sequences corresponding to a CD274 gene or a portion thereof; wherein the selectively enriching produces an enriched sample. In some embodiments, the selectively enriching comprises: (a) combining one or more bait molecules with the sample, thereby hybridizing the one or more bait molecules to one or more nucleic acid molecules in the sample comprising nucleotide sequences corresponding to a CD274 gene or a portion thereof and producing nucleic acid hybrids; and (b) isolating the nucleic acid hybrids to produce the enriched sample. In some embodiments, which may be combined with any of the preceding aspects or embodiments, the one or more bait molecules comprise a capture nucleic acid molecule configured to hybridize to a nucleotide sequence corresponding to a CD274 gene or a portion thereof. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, which may be combined with any of the preceding aspects or embodiments, the one or more bait molecules are conjugated to an affinity reagent or to a detection reagent. In some embodiments, the affinity reagent is an antibody, an antibody fragment, or biotin, or wherein the detection reagent is a fluorescent marker. In some embodiments, which may be combined with any of the preceding aspects or embodiments, the capture nucleic acid molecule comprises a DNA, RNA, or mixed DNA/RNA molecule. In some embodiments, which may be combined with any of the preceding aspects or embodiments, the selectively enriching comprises amplifying the one or more nucleic acid molecules comprising nucleotide sequences corresponding to a CD274 gene or a portion thereof using a polymerase chain reaction (PCR) to produce an enriched sample. In some embodiments, which may be combined with any of the preceding aspects or embodiments, the method further comprises sequencing the enriched sample. In some embodiments, which may be combined with any of the preceding aspects or embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample.

In some embodiments, which may be combined with any of the preceding aspects or embodiments, the analyzing step comprises: (a) generating a copy number model based on the plurality of sequence reads; and (b) determining a CD274 gene copy number based on the copy number model. In some embodiments, the copy number model is generated by: (a) aligning the plurality of sequence reads against a reference genome; (b) normalizing sequence coverage distribution of the aligned plurality of sequence reads against a control sample; and (c) generating segmentation data for the normalized plurality of sequence reads. In some embodiments, which may be combined with any of the preceding aspects or embodiments, the analyzing step comprises: determining coverage ratio data, allele fraction data, and segmentation data for one or more gene loci within one or more subgenomic intervals of the plurality of sequence reads, wherein the one or more gene loci comprise CD274; identifying a plurality of segments based on the segmentation data; determining copy numbers for the plurality of segments based on the coverage ratio data, the allele fraction data, the segmentation data, and a copy number model; detecting the presence or absence of a CD274 copy number gain based on a copy number of a segment of the plurality of segments corresponding to CD274. In some embodiments, a CD274 copy number gain is detected when the copy number for the segment of the plurality of segments corresponding to CD274 is greater than or equal to ploidy of the sample. In some embodiments, a CD274 copy number gain is detected when the copy number for the segment of the plurality of segments corresponding to CD274 is greater than ploidy of the sample. In some embodiments, the coverage ratio data is determined by aligning a plurality of sequence reads from the sample and from a control sample to a reference genome, and determining a number of sequence reads that overlap each of the one or more gene loci within the one or more subgenomic intervals in the sample and in the control sample. In some embodiments, the control sample is a paired normal sample, a process-matched control sample, or a panel of normal control sample. In some embodiments, the copy number model: (a) predicts a copy number for CD274 based on coverage ratio data and allele fraction data; (b) predicts a sample purity and ploidy for the sample; (c) outputs segmentation data; and any combination thereof. In some embodiments, the segmentation data is generated by aligning a plurality of sequence reads from the sample to a reference genome, and processing the aligned sequence read data, coverage ratio data, and allele fraction data to determine a number of segments required to account for the aligned sequence read data. In some embodiments, each segment has the same copy number. In some embodiments, the method comprises processing the aligned sequence read data, coverage ratio data, and allele fraction data using a pruned exact linear time (PELT) method to determine a number of segments required to account for the aligned sequence read data, wherein each segment has a same copy number. In some embodiments, the reference genome is a human genome. In some embodiments, the segmentation data is generated using a circular binary segmentation (CBS) method. In some embodiments, the CD274 gene copy number is determined based on a summary statistic of the copy number of all genomic segments overlapping a CD274 gene. In some embodiments, the summary statistic comprises the mean, median, maximum or mode of the copy number of all genomic segments overlapping a CD274 gene. In some embodiments, the copy number model is a genome-wide copy number model. In some embodiments, the segmentation comprises whole-genome segmentation. In some embodiments, each segment has an equal copy number. In some embodiments, the method further comprises calling a CD274 copy number gain based on a copy number for the segment of the plurality of segments corresponding to CD274 that is greater than or equal to ploidy of the sample, or based on detecting the presence or absence of a CD274 copy number gain. In some embodiments, the method further comprises calling a CD274 copy number gain based on a copy number for the segment of the plurality of segments corresponding to CD274 that is greater than ploidy of the sample.

In some embodiments, which may be combined with any of the preceding aspects or embodiments, the CD274 gene copy number gain is detected by fluorescence in situ hybridization (FISH) based on a ratio of fluorescence signal from a FISH probe that binds to CD274 and from a control FISH probe. In some embodiments, the control FISH probe is a centromere enumeration probe for chromosome 9 (CEP9). In some embodiment, the CD274 gene copy number gain comprises a ratio of fluorescence signal from a FISH probe that binds to CD274 to fluorescence signal from the control FISH probe of any of at least 2, at least 3, at least 4, at least 5, at least 6 or more.

In some embodiments, which may be combined with any of the preceding aspects or embodiments, the high TMB is detected in a sample from the individual by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next-generation sequencing (NGS). In some embodiments, which may be combined with any of the preceding aspects or embodiments, TMB is assessed based on about 0.79 megabases (Mb) of sequenced DNA. In some embodiments, which may be combined with any of the preceding aspects or embodiments, TMB is assessed based on about 0.80 Mb of sequenced DNA. In some embodiments, which may be combined with any of the preceding aspects or embodiments, TMB is assessed based on between about 0.83 Mb and about 1.14 Mb of sequenced DNA. In some embodiments, which may be combined with any of the preceding aspects or embodiments, TMB is assessed based on about 1.1 Mb of sequenced DNA. In some embodiments, which may be combined with any of the preceding aspects or embodiments, TMB is assessed based on up to about 1.24 Mb of sequenced DNA. In some embodiments, which may be combined with any of the preceding aspects or embodiments, TMB is assessed based on up to about 1.1 Mb of sequenced DNA. In some embodiments, which may be combined with any of the preceding aspects or embodiments, the high TMB comprises a TMB of at least about 5 mutations/Megabase (mut/Mb) or at least about 10 mut/Mb. In some embodiments, which may be combined with any of the preceding aspects or embodiments, the cancer comprises a TMB of any of at least about 5 mut/Mb, at least about 10 mut/Mb, at least about 20 mut/Mb, at least about 30 mut/Mb, at least about 40 mut/Mb, at least about 50 mut/Mb, at least about 60 mut/Mb, at least about 70 mut/Mb, at least about 80 mut/Mb, at least about 90 mut/Mb, at least about 100 mut/Mb, at least about 110 mut/Mb, at least about 120 mut/Mb, at least about 130 mut/Mb, at least about 140 mut/Mb, at least about 150 mut/Mb, or more.

In some embodiments, which may be combined with any of the preceding aspects or embodiments, the method further comprises generating a report, wherein the report: (a) indicates the presence of the CD274 gene copy number gain and high TMB in the one or more samples from the individual; and/or (b) indicates a treatment or one or more treatment options identified or selected for the individual based, at least in part, on the presence of the CD274 gene copy number gain and high TMB in the one or more samples from the individual, wherein the treatment or the one or more treatment options comprise an immunotherapy. In some embodiments, which may be combined with any of the preceding aspects or embodiments, the method further comprises generating a molecular profile for the individual, based, at least in part, on detecting or acquiring knowledge of the CD274 gene copy number gain and/or high TMB. In some embodiments, the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile for the individual further comprises results from a nucleic acid sequencing-based test. In some embodiments, the method further comprises selecting a treatment, administering a treatment, or applying a treatment to the individual based on the generated molecular profile, wherein the treatment comprises an immunotherapy. In some embodiments, which may be combined with any of the preceding aspects or embodiments, the method further comprises generating a report, wherein the report comprises the molecular profile for the individual. In some embodiments, the report further comprises information on a treatment or one or more treatment options identified or selected for the individual based, at least in part, on the molecular profile for the individual, wherein the treatment or one or more treatment options comprise an immunotherapy. In some embodiments, which may be combined with any of the preceding aspects or embodiments, the method further comprises providing the report to the individual, a caregiver, a healthcare provider, a physician, an oncologist, an electronic medical record system, a hospital, a clinic, a third-party payer, an insurance company, or a government office.

In some embodiments, which may be combined with any of the preceding aspects or embodiments, the method further comprises assessing microsatellite instability status of the cancer in a sample from the individual. In some embodiments, which may be combined with any of the preceding aspects or embodiments, the report further indicates the microsatellite instability status of the cancer. In some embodiments, which may be combined with any of the preceding aspects or embodiments, the molecular profile further indicates the microsatellite instability status of the cancer. In some embodiments, which may be combined with any of the preceding aspects or embodiments, microsatellite instability is assessed by sequencing, a polymerase chain reaction (PCR) amplification technique, a non-PCR amplification technique, an isothermal amplification technique, a capillary electrophoresis method, immunohistochemistry, and any combination thereof. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next-generation sequencing (NGS). In some embodiments, which may be combined with any of the preceding aspects or embodiments, the cancer has high microsatellite instability (MSI-high). In some embodiments, which may be combined with any of the preceding aspects or embodiments, the cancer is microsatellite stable.

In some embodiments, which may be combined with any of the preceding aspects or embodiments, the method further comprises assessing expression of PD-L1 protein in a sample from the individual. In some embodiments, which may be combined with any of the preceding aspects or embodiments, the report further indicates the PD-L1 protein expression status of the cancer. In some embodiments, which may be combined with any of the preceding aspects or embodiments, the molecular profile further indicates the PD-L1 protein expression status of the cancer. In some embodiments, which may be combined with any of the preceding aspects or embodiments, PD-L1 protein expression is determined using an immunohistochemistry assay. In some embodiments, the immunohistochemistry assay is a DAKO PD-L1 22C3 assay. In some embodiments, which may be combined with any of the preceding aspects or embodiments, PD-L1 expression is assessed based on a tumor proportion score (TPS). In some embodiments, the cancer is PD-L1 positive. In some embodiments, the cancer has a TPS of at least about 1%. In some embodiments, the cancer has a TPS of between about 1% and about 49%. In some embodiments, the cancer has a TPS of at least about 50%. In some embodiments, the cancer has a TPS of at least about 1%, at least about 25%, at least about 50%, or at least about 75%. In some embodiments, the cancer has a TPS of at least about 1%, at least about 5%, at least about 10%, at least about 20%, at least about 30%, at least about 40%, at least about 50%, at least about 60%, at least about 70%, at least about 80%, at least about 90%, at least about 95%, at least about 99%, or 100%. In some embodiments, the cancer is PD-L1 negative. In some embodiments, the cancer has a TPS of less than 1%. In some embodiments, which may be combined with any of the preceding aspects or embodiments, PD-L1 expression is assessed based on a combined positive score (CPS). In some embodiments, the cancer is PD-L1 positive. In some embodiments, the cancer has a CPS of at least about 1 or at least about 10. In some embodiments, the cancer is PD-L1 negative. In some embodiments, the cancer has a CPS of less than 1. In some embodiments, the immunohistochemistry assay is a VENTANA SP 142 assay. In some embodiments, which may be combined with any of the preceding aspects or embodiments, PD-L1 expression is assessed based on the proportion of tumor area occupied by PD-L1-expressing tumor-infiltrating immune cells of any intensity (IC), or the percentage of PD-L1-expressing tumor cells of any intensity (TC). In some embodiments, the cancer is PD-L1 positive. In some embodiments, the cancer has a TC or IC of at least about 1%. In some embodiments, the cancer is PD-L1 negative. In some embodiments, the cancer has a TC or IC of less than 1%.

In some embodiments, which may be combined with any of the preceding aspects or embodiments, the method further comprises acquiring knowledge of or detecting in a sample from the individual a base substitution, a short insertion/deletion (indel), a copy number alteration, or a genomic rearrangement in one or more genes; optionally wherein the one or more genes comprise CD274, EGFR and/or ALK.

In some embodiments, which may be combined with any of the preceding aspects or embodiments, the individual is a human.

In some embodiments, which may be combined with any of the preceding aspects or embodiments, the cancer is a carcinoma, a sarcoma, a lymphoma, a leukemia, a myeloma, a germ cell cancer, or a blastoma. In some embodiments, which may be combined with any of the preceding aspects or embodiments, the cancer is a solid tumor. In some embodiments, which may be combined with any of the preceding aspects or embodiments, the cancer is a hematologic malignancy. In some embodiments, which may be combined with any of the preceding aspects or embodiments, the cancer is a B cell cancer, multiple myeloma, melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, which may be combined with any of the preceding aspects or embodiments, the cancer is a non-small cell lung cancer. In some embodiments, which may be combined with any of the preceding aspects or embodiments, the cancer is a non-squamous non-small cell lung cancer. In some embodiments, which may be combined with any of the preceding aspects or embodiments, the CD274 gene copy number gain in a sample from the individual comprises a CD274 gene copy number of at least +2, as compared to the ploidy of the sample from the individual, optionally wherein the ploidy of the sample is diploid; and wherein the cancer comprises a TMB of at least about 10 mut/Mb. In some embodiments, which may be combined with any of the preceding aspects or embodiments, the CD274 gene copy number gain in a sample from the individual comprises a CD274 gene copy number of at least 4, wherein the ploidy of the sample from the individual is diploid; and wherein the cancer comprises a TMB of at least about 10 mut/Mb. In some embodiments, which may be combined with any of the preceding aspects or embodiments, administering a treatment comprising an immunotherapy to the individual results in survival of the individual for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 25 months, or more, measured from the start of treatment with the immunotherapy. In some embodiments, which may be combined with any of the preceding aspects or embodiments, administering a treatment comprising an immunotherapy to a plurality of individuals results in a median overall survival of the individuals in the plurality of at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 25 months, or more, measured from the start of treatment with the immunotherapy. In some embodiments, the immunotherapy is an immune checkpoint inhibitor.

In some embodiments, which may be combined with any of the preceding aspects or embodiments, the cancer is a Stage I, Stage II, Stage III, Stage IV, or unknown stage cancer. In some embodiments, which may be combined with any of the preceding aspects or embodiments, the individual has a history smoking, or does not have a history of smoking. In some embodiments, which may be combined with any of the preceding aspects or embodiments, the cancer is metastatic. In some embodiments, which may be combined with any of the preceding aspects or embodiments, the individual has an Eastern Cooperative Oncology Group status of any of 0, 1, 2, 3, or more. In some embodiments, which may be combined with any of the preceding aspects or embodiments, the cancer does not comprise any oncogenic alterations in an EGFR and/or an ALK gene. In some embodiments, which may be combined with any of the preceding aspects or embodiments, the cancer is wild type for oncogenic alterations in an EGFR and/or an ALK gene. In some embodiments, the oncogenic alterations comprise base substitutions, insertions/deletions, copy number alterations, or rearrangements. In some embodiments, which may be combined with any of the preceding aspects or embodiments, the individual was previously treated with a first-line treatment for the cancer with an anti-VEGF chemotherapy combination treatment, an EGFR tyrosine kinase inhibitor, a platinum-based chemotherapy, or a single agent chemotherapy.

In some embodiments, which may be combined with any of the preceding aspects or embodiments, the immunotherapy is an immune checkpoint inhibitor, a cancer vaccine, a cell-based therapy, a T cell receptor (TCR)-based therapy, an adjuvant immunotherapy, a cytokine immunotherapy, or an oncolytic virus therapy. In some embodiments, which may be combined with any of the preceding aspects or embodiments, the immunotherapy is a monotherapy. In some embodiments, which may be combined with any of the preceding aspects or embodiments, the immune checkpoint inhibitor is a first-line immune checkpoint inhibitor. In some embodiments, which may be combined with any of the preceding aspects or embodiments, the immune checkpoint inhibitor is a second-line immune checkpoint inhibitor. In some embodiments, which may be combined with any of the preceding aspects or embodiments, the immune checkpoint inhibitor is a PD-1- or a PD-L1-targeted agent. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab.

In some embodiments, which may be combined with any of the preceding aspects or embodiments, the cancer in the individual has not been previously treated with an immune checkpoint inhibitor.

In some embodiments, which may be combined with any of the preceding aspects or embodiments, the treatment or the one or more treatment options comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the method further comprises obtaining the one or more samples from the individual. In some embodiments, the one or more samples are obtained or derived from the cancer. In some embodiments, one or more samples from the individual comprise a tissue biopsy sample, a liquid biopsy sample, or a normal control. In some embodiments, one or more samples from the individual are from a tumor biopsy, tumor specimen, or circulating tumor cell. In some embodiments, one or more samples from the individual are a liquid biopsy sample comprising blood, plasma, cerebrospinal fluid, sputum, stool, urine, or saliva. In some embodiments, one or more samples from the individual comprise cells and/or nucleic acids from the cancer. In some embodiments, one or more samples from the individual comprise at least 20% tumor cell nuclear area. In some embodiments, one or more samples from the individual comprise mRNA, DNA, circulating tumor DNA (ctDNA), cell-free DNA, or cell-free RNA from the cancer. In some embodiments, one or more samples from the individual are a liquid biopsy sample comprising circulating tumor cells (CTCs). In some embodiments, one or more samples from the individual are a liquid biopsy sample comprising cell-free DNA (cfDNA), circulating tumor DNA (ctDNA), or any combination thereof.

In another aspect, provided herein is a system for identifying an individual having a cancer who may benefit from a treatment comprising an anti-cancer therapy, the system comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from one or more samples obtained from an individual having a cancer; (b) analyze the plurality of sequence reads for the presence of a CD274 gene copy number gain and a high TMB; and (c) detect, based on the analyzing, the presence of the CD274 gene copy number gain and high TMB in the one or more samples; wherein detecting the CD274 gene copy number gain and high TMB identifies the individual as one who may benefit from a treatment comprising an anti-cancer therapy.

In another aspect, provided herein is a non-transitory computer readable storage medium comprising one or more programs executable by one or more computer processors for performing a method for identifying an individual having a cancer who may benefit from a treatment comprising an anti-cancer therapy, the method comprising: (a) obtaining, using the one or more processors, a plurality of sequence reads of one or more nucleic acid molecules, wherein the one or more nucleic acid molecules are derived from one or more samples obtained from an individual having a cancer; (b) analyzing, using the one or more processors, the plurality of sequence reads for the presence of a CD274 gene copy number gain and a high TMB; and (c) detecting, using the one or more processors and based on the analyzing, the CD274 gene copy number gain and high TMB in the one or more samples; wherein detecting the CD274 gene copy number gain and high TMB identifies the individual as one who may benefit from a treatment comprising an anti-cancer therapy.

In some embodiments, which may be combined with any of the preceding aspects or embodiments, the anti-cancer therapy is any anti-cancer therapy or agent known in the art or provided herein. In some embodiments, which may be combined with any of the preceding aspects or embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a system for identifying an individual having a cancer who may benefit from a treatment comprising an immunotherapy, the system comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from one or more samples obtained from an individual having a cancer; (b) analyze the plurality of sequence reads for the presence of a CD274 gene copy number gain and a high TMB; and (c) detect, based on the analyzing, the presence of the CD274 gene copy number gain and high TMB in the one or more samples; wherein detecting the CD274 gene copy number gain and high TMB identifies the individual as one who may not benefit from a treatment comprising an anti-cancer therapy and may benefit from an immunotherapy. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a non-transitory computer readable storage medium comprising one or more programs executable by one or more computer processors for performing a method for identifying an individual having a cancer who may benefit from a treatment comprising an immunotherapy, the method comprising: (a) obtaining, using the one or more processors, a plurality of sequence reads of one or more nucleic acid molecules, wherein the one or more nucleic acid molecules are derived from one or more samples obtained from an individual having a cancer; (b) analyzing, using the one or more processors, the plurality of sequence reads for the presence of a CD274 gene copy number gain and a high TMB; and (c) detecting, using the one or more processors and based on the analyzing, the CD274 gene copy number gain and high TMB in the one or more samples; wherein detecting the CD274 gene copy number gain and high TMB identifies the individual as one who may not benefit from a treatment comprising an anti-cancer therapy and may benefit from an immunotherapy. In some embodiments, the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

In another aspect, provided herein is a system for identifying an individual having a cancer who may benefit from a treatment comprising an immunotherapy, the system comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from one or more samples obtained from an individual having a cancer; (b) analyze the plurality of sequence reads for the presence of a CD274 gene copy number gain and a high TMB; and (c) detect, based on the analyzing, the presence of the CD274 gene copy number gain and high TMB in the one or more samples; wherein detecting the CD274 gene copy number gain and high TMB identifies the individual as one who may benefit from a treatment comprising an immunotherapy.

In another aspect, provided herein is a non-transitory computer readable storage medium comprising one or more programs executable by one or more computer processors for performing a method for identifying an individual having a cancer who may benefit from a treatment comprising an immunotherapy, the method comprising: (a) obtaining, using the one or more processors, a plurality of sequence reads of one or more nucleic acid molecules, wherein the one or more nucleic acid molecules are derived from one or more samples obtained from an individual having a cancer; (b) analyzing, using the one or more processors, the plurality of sequence reads for the presence of a CD274 gene copy number gain and a high TMB; and (c) detecting, using the one or more processors and based on the analyzing, the CD274 gene copy number gain and high TMB in the one or more samples; wherein detecting the CD274 gene copy number gain and high TMB identifies the individual as one who may benefit from a treatment comprising an immunotherapy.

In some embodiments, which may be combined with any of the preceding aspects or embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next-generation sequencing (NGS).

In some embodiments, which may be combined with any of the preceding aspects or embodiments, the analyzing step comprises: (a) generating a copy number model based on the plurality of sequence reads; and (b) determining a CD274 gene copy number based on the copy number model. In some embodiments, the copy number model is generated by: (a) aligning the plurality of sequence reads against a reference genome; (b) normalizing sequence coverage distribution of the aligned plurality of sequence reads against a control sample; and (c) generating segmentation data for the normalized plurality of sequence reads. In some embodiments, which may be combined with any of the preceding aspects or embodiments, the analyzing step comprises: determining coverage ratio data, allele fraction data, and segmentation data for one or more gene loci within one or more subgenomic intervals of the plurality of sequence reads, wherein the one or more gene loci comprise CD274; identifying a plurality of segments based on the segmentation data; determining copy numbers for the plurality of segments based on the coverage ratio data, the allele fraction data, the segmentation data, and a copy number model; detecting the presence or absence of a CD274 copy number gain based on a copy number of a segment of the plurality of segments corresponding to CD274. In some embodiments, a CD274 copy number gain is detected when the copy number for the segment of the plurality of segments corresponding to CD274 is greater than or equal to ploidy of the sample. In some embodiments, a CD274 copy number gain is detected when the copy number for the segment of the plurality of segments corresponding to CD274 is greater than ploidy of the sample. In some embodiments, the coverage ratio data is determined by aligning a plurality of sequence reads from the sample and from a control sample to a reference genome, and determining a number of sequence reads that overlap each of the one or more gene loci within the one or more subgenomic intervals in the sample and in the control sample. In some embodiments, the control sample is a paired normal sample, a process-matched control sample, or a panel of normal control sample. In some embodiments, the copy number model: (a) predicts a copy number for CD274 based on coverage ratio data and allele fraction data; (b) predicts a sample purity and ploidy for the sample; (c) outputs segmentation data; and any combination thereof. In some embodiments, the segmentation data is generated by aligning a plurality of sequence reads from the sample to a reference genome, and processing the aligned sequence read data, coverage ratio data, and allele fraction data to determine a number of segments required to account for the aligned sequence read data. In some embodiments, each segment has the same copy number. In some embodiments, the analyzing comprises processing the aligned sequence read data, coverage ratio data, and allele fraction data using a pruned exact linear time (PELT) method to determine a number of segments required to account for the aligned sequence read data, wherein each segment has a same copy number. In some embodiments, the reference genome is a human genome. In some embodiments, the segmentation data is generated using a circular binary segmentation (CBS) method. In some embodiments, the CD274 gene copy number is determined based on a summary statistic of the copy number of all genomic segments overlapping a CD274 gene. In some embodiments, the summary statistic comprises the mean, median, maximum or mode of the copy number of all genomic segments overlapping a CD274 gene. In some embodiments, the copy number model is a genome-wide copy number model. In some embodiments, the segmentation comprises whole-genome segmentation. In some embodiments, each segment has an equal copy number. In some embodiments, the analyzing further comprises calling a CD274 copy number gain based on a copy number for the segment of the plurality of segments corresponding to CD274 that is greater than or equal to ploidy of the sample, or based on detecting the presence or absence of a CD274 copy number gain. In some embodiments, the analyzing further comprises calling a CD274 copy number gain based on a copy number for the segment of the plurality of segments corresponding to CD274 is greater than ploidy of the sample.

In some embodiments, which may be combined with any of the preceding aspects or embodiments, the CD274 gene copy number gain comprises a CD274 gene copy number of any of at least +1, at least +2, at least +3, at least +4, at least +5, at least +6, at least +7, at least +8, or more, as compared to the ploidy of a sample from the individual. In some embodiments, which may be combined with any of the preceding aspects or embodiments, the CD274 gene copy number gain comprises a CD274 gene copy number of any of at least +2, at least +3, at least +4, at least +5, at least +6, at least +7, at least +8, or more, as compared to the ploidy of a sample from the individual. In some embodiments, which may be combined with any of the preceding aspects or embodiments, the CD274 gene copy number gain comprises a CD274 gene copy number of at least +2, as compared to the ploidy of a sample from the individual. In some embodiments, which may be combined with any of the preceding aspects or embodiments, the ploidy of the sample from the individual is monoploid, diploid, triploid, or tetraploid.

In some embodiments, which may be combined with any of the preceding aspects or embodiments, the CD274 gene copy number gain in a sample from the individual comprises a CD274 gene copy number of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is monoploid; the CD274 gene copy number gain in a sample from the individual comprises a CD274 gene copy number of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is diploid; the CD274 gene copy number gain in a sample from the individual comprises a CD274 gene copy number of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is triploid; or the CD274 gene copy number gain in a sample from the individual comprises a CD274 gene copy number of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is tetraploid. In some embodiments, which may be combined with any of the preceding aspects or embodiments, the CD274 gene copy number is any of at least four, at least five, at least six, or more. In some embodiments, which may be combined with any of the preceding aspects or embodiments, the CD274 copy number is at least four.

In some embodiments, which may be combined with any of the preceding aspects or embodiments, TMB is assessed based on about 0.79 megabases (Mb) of sequenced DNA. In some embodiments, which may be combined with any of the preceding aspects or embodiments, TMB is assessed based on about 0.80 Mb of sequenced DNA. In some embodiments, which may be combined with any of the preceding aspects or embodiments, TMB is assessed based on between about 0.83 Mb and about 1.14 Mb of sequenced DNA. In some embodiments, which may be combined with any of the preceding aspects or embodiments, TMB is assessed based on about 1.1 Mb of sequenced DNA. In some embodiments, which may be combined with any of the preceding aspects or embodiments, TMB is assessed based on up to about 1.24 Mb of sequenced DNA. In some embodiments, which may be combined with any of the preceding aspects or embodiments, TMB is assessed based on up to about 1.1 Mb of sequenced DNA. In some embodiments, which may be combined with any of the preceding aspects or embodiments, the high TMB comprises a TMB of at least about 5 mutations/Megabase (mut/Mb), or at least about 10 mut/Mb. In some embodiments, which may be combined with any of the preceding aspects or embodiments, the cancer comprises a TMB of any of at least about 5 mut/Mb, at least about 10 mut/Mb, at least about 20 mut/Mb, at least about 30 mut/Mb, at least about 40 mut/Mb, at least about 50 mut/Mb, at least about 60 mut/Mb, at least about 70 mut/Mb, at least about 80 mut/Mb, at least about 90 mut/Mb, at least about 100 mut/Mb, at least about 110 mut/Mb, at least about 120 mut/Mb, at least about 130 mut/Mb, at least about 140 mut/Mb, at least about 150 mut/Mb, or more.

In some embodiments, which may be combined with any of the preceding aspects or embodiments, the individual is administered a treatment based, at least in part, on detecting the CD274 gene copy number gain and high TMB in the one or more samples.

In some embodiments, which may be combined with any of the preceding aspects or embodiments, the one or more program instructions when executed by the one or more processors are further configured to generate, based at least in part on the detecting, a molecular profile for the individual.

In some embodiments, which may be combined with any of the preceding aspects or embodiments, the method further comprises generating, based at least in part on the detecting, a molecular profile for the individual.

In some embodiments, which may be combined with any of the preceding aspects or embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, which may be combined with any of the preceding aspects or embodiments, the molecular profile further comprises results from a nucleic acid sequencing-based test.

In some embodiments, which may be combined with any of the preceding aspects or embodiments, the one or more program instructions when executed by the one or more processors are further configured to generate a report, wherein the report indicates the presence of the CD274 gene copy number gain and high TMB in the one or more samples.

In some embodiments, which may be combined with any of the preceding aspects or embodiments, the one or more program instructions when executed by the one or more processors are further configured to generate a report, wherein the report comprises the molecular profile.

In some embodiments, which may be combined with any of the preceding aspects or embodiments, the method further comprises generating a report, wherein the report indicates the presence of the CD274 gene copy number gain and high TMB in the one or more samples.

In some embodiments, which may be combined with any of the preceding aspects or embodiments, the method further comprises generating a report, wherein the report comprises the molecular profile.

In some embodiments, which may be combined with any of the preceding aspects or embodiments, the one or more program instructions when executed by the one or more processors are further configured to transmit the report to the individual, a caregiver, a healthcare provider, a physician, an oncologist, an electronic medical record system, a hospital, a clinic, a third-party payer, an insurance company, or a government office.

In some embodiments, which may be combined with any of the preceding aspects or embodiments, the method further comprises transmitting the report to the individual, a caregiver, a healthcare provider, a physician, an oncologist, an electronic medical record system, a hospital, a clinic, a third-party payer, an insurance company, or a government office.

In some embodiments, which may be combined with any of the preceding aspects or embodiments, the report is transmitted via a computer network or a peer-to-peer connection.

In some embodiments, which may be combined with any of the preceding aspects or embodiments, the individual is administered a treatment based, at least in part, on the report.

In some embodiments, which may be combined with any of the preceding aspects or embodiments, the individual is administered a treatment based, at least in part, on the molecular profile for the individual.

In some embodiments, which may be combined with any of the preceding aspects or embodiments, the treatment comprises an immunotherapy. In some embodiments, the immunotherapy is an immune checkpoint inhibitor, a cancer vaccine, a cell-based therapy, a T cell receptor (TCR)-based therapy, an adjuvant immunotherapy, a cytokine immunotherapy, or an oncolytic virus therapy. In some embodiments, the immunotherapy is a monotherapy. In some embodiments, the immune checkpoint inhibitor is a first-line immune checkpoint inhibitor. In some embodiments, the immune checkpoint inhibitor is a second-line immune checkpoint inhibitor. In some embodiments, the immune checkpoint inhibitor is a PD-1- or a PD-L1-targeted agent. In some embodiments, the immune checkpoint inhibitor is a PD-1 inhibitor, a PD-L1-inhibitor, or a CTLA-4 inhibitor. In some embodiments, the immune checkpoint inhibitor comprises one or more of nivolumab, pembrolizumab, cemiplimab, dostarlimab, atezolizumab, avelumab, durvalumab, or ipilimumab.

In some embodiments, which may be combined with any of the preceding aspects or embodiments, the individual is a human.

In some embodiments, which may be combined with any of the preceding aspects or embodiments, the cancer is a carcinoma, a sarcoma, a lymphoma, a leukemia, a myeloma, a germ cell cancer, or a blastoma. In some embodiments, which may be combined with any of the preceding aspects or embodiments, the cancer is a solid tumor or a hematologic malignancy. In some embodiments, which may be combined with any of the preceding aspects or embodiments, the cancer is a B cell cancer, multiple myeloma, melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, which may be combined with any of the preceding aspects or embodiments, the cancer is a non-small cell lung cancer. In some embodiments, which may be combined with any of the preceding aspects or embodiments, the cancer is a non-squamous non-small cell lung cancer. In some embodiments, which may be combined with any of the preceding aspects or embodiments, the CD274 gene copy number gain comprises a CD274 gene copy number of at least +2, as compared to the ploidy of a sample from the individual; and wherein the cancer comprises a TMB of at least about 10 mut/Mb. In some embodiments, which may be combined with any of the preceding aspects or embodiments, the CD274 gene copy number gain comprises a CD274 gene copy number of at least 4, wherein the ploidy of a sample from the individual is diploid; and wherein the cancer comprises a TMB of at least about 10 mut/Mb.

In some embodiments, which may be combined with any of the preceding aspects or embodiments, the cancer does not comprise any oncogenic alterations in an EGFR and/or an ALK gene. In some embodiments, which may be combined with any of the preceding aspects or embodiments, the cancer is wild type for oncogenic alterations in an EGFR and/or an ALK gene. In some embodiments, the oncogenic alterations comprise base substitutions, insertions/deletions, copy number alterations, or rearrangements.

In another aspect, provided herein is an anti-cancer therapy for use in a method of treating or delaying progression of cancer in an individual, wherein the method comprises administering the anti-cancer therapy to an individual having a cancer, wherein a CD274 gene copy number gain and a high TMB are detected in one or more samples obtained from the individual.

In another aspect, provided herein is an anti-cancer therapy for use in the manufacture of a medicament for treating or delaying progression of cancer in an individual, wherein the medicament is to be administered to an individual having a cancer, wherein a CD274 gene copy number gain and a high TMB are detected in one or more samples obtained from the individual.

In another aspect, provided herein is an immunotherapy for use in a method of treating or delaying progression of cancer in an individual, wherein the method comprises administering the immunotherapy to an individual having a cancer, wherein a CD274 gene copy number gain and a high TMB are detected in one or more samples obtained from the individual.

In another aspect, provided herein is an immunotherapy for use in the manufacture of a medicament for treating or delaying progression of cancer in an individual, wherein the medicament is to be administered to an individual having a cancer, wherein a CD274 gene copy number gain and a high TMB are detected in one or more samples obtained from the individual.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows the demographic and clinical features of non-squamous non-small cell lung cancer (NSCLC) patients in a real-world clinico-genomic cohort in the study described in Example 1. ICI=immune checkpoint inhibitor; IQR=inter-quartile range; ECOG=Eastern Cooperative Oncology Group.

FIGS. 2A-2D show the overall survival (OS) of non-squamous NSCLC patients in the study described in Example 1. OS was measured from the start of second-line immune checkpoint inhibitor (ICI) monotherapy, and was stratified by CD274 copy number (CN) relative to specimen ploidy. FIG. 2A shows the OS for patients with a CD274 CN≥specimen ploidy +1 compared to patients with a CD274 CN<specimen ploidy +1. The median OS (mOS) of patients with a CD274 CN≥specimen ploidy +1(N=93) was 9.6 (7.6-16.2) months, while patients with a CD274 CN<specimen ploidy +1 (N=528) had a mOS of 8.8 (6.9-11.2) months. The hazard ratio (HR) for the CD274 CN≥specimen ploidy +1 group was 0.8 (95% confidence interval [CI]=0.6-1.0, p=0.09). FIG. 2B shows the OS for patients with a CD274 CN≥specimen ploidy +2 compared to patients with a CD274 CN<specimen ploidy +2. The mOS of patients with a CD274 CN≥specimen ploidy +2 (N=29) was 16.1 (CI=8.9-37.3) months, while patients with a CD274 CN<specimen ploidy +2 (N=592) had a mOS of 8.6 (CI=7.1-10.9) months. The HR for the CD274 CN≥specimen ploidy +2 group was 0.6 (CI=0.4-1.0; p=0.05). FIG. 2C shows the OS for patients with a CD274 CN>specimen ploidy +3 compared to patients with a CD274 CN<specimen ploidy +3. The mOS of patients with a CD274 CN≥specimen ploidy +3 (N=15) was 14.8 (CI=8.9-NA) months, while patients with a CD274 CN<specimen ploidy +3 (N=606) had a mOS of 8.8 (CI=7.3-11) months. The HR for the CD274 CN≥specimen ploidy +3 group was 0.8 (CI=0.4-1.5; p=0.5). FIG. 2D shows the OS for patients with a CD274 CN≥specimen ploidy +4 compared to patients with a CD274 CN<specimen ploidy +4. The mOS of patients with a CD274 CN≥specimen ploidy +4 (N=9) was 8.94 (CI=3.8-NA) months, while patients with a CD274 CN<specimen ploidy +4 (N=612) had a mOS of 8.9 (CI=7.3-11.2) months. The HR for the CD274 CN≥specimen ploidy +4 group was 0.8 (CI=0.4-1.9; p=0.7).

FIG. 3 shows a comparison of demographic and clinical characteristics between non-squamous NSCLC patients in the study described in Example 1 with a CD274 CN≥specimen ploidy +2 and with a CD274 CN<specimen ploidy +2.

FIG. 4 shows a comparison of ICI therapy-associated biomarkers between non-squamous NSCLC patients in the study described in Example 1 with CD274 CN≥specimen ploidy +2 and with CD274 CN<specimen ploidy +2. The p values were estimated using the two-sided Fisher's exact test. TPS=tumor proportion score; TMB=tumor mutational burden; MSI=microsatellite instability.

FIG. 5 shows the distribution of PD-L1 immunohistochemistry (IHC) status of non-squamous NSCLC patients in the study described in Example 1 across the indicated CD274 CN≥gain and loss thresholds. A total of 124 out of 621 patients in the clinico-genomic cohort described in Example 1 had data on PD-L1 IHC status, as measured by the tumor proportion score (TPS).

FIG. 6 shows the OS of ICI-treated non-squamous NSCLC patients in the study described in Example 1, stratified by tumor mutational burden (TMB; at a threshold of 10 mut/Mb) and CD274 CN group (at a copy number gain threshold of 2). The mOS of patients with CD274 CN<specimen ploidy +2 and TMB low (N=330) was 7.7 (CI=6.3-10.9) months, the mOS of patients with CD274 CN<specimen ploidy +2 and TMB high (N=262) was 9.5 (CI=7.1-13.2) months, the mOS of patients with CD274 CN≥specimen ploidy +2 and TMB low (N=9) was 9.3 (CI=1.3-NA) months, and the mOS of patients with CD274 CN≥specimen ploidy +2 and TMB high (N=20) was 24.9 (CI=11.1-NA) months (p=0.04).

FIG. 7 shows the distribution of PD-L1 IHC status of non-squamous NSCLC patients in the study described in Example 1 across TMB (at a threshold of 10 mut/Mb) and CD274 CN (at a CN gain threshold of 2) groups. A total of 124 out of 621 patients in the clinico-genomic cohort described in Example 1 had data on PD-L1 IHC status, as determined by TPS.

FIGS. 8A-8C show the OS of non-squamous NSCLC patients in the study described in Example 1. The OS is shown from start of second-line ICI monotherapy, and is stratified by CD274 CN loss relative to specimen ploidy. FIG. 8A shows the OS for patients with a CD274 CN≤specimen ploidy −1 compared to patients with a CD274 CN>specimen ploidy −1. The mOS of patients with a CD274 CN>specimen ploidy −1 (N=322) was 9.6 (CI=7.9-12.8) months, while patients with a CD274 CN<specimen ploidy −1 (N=299) had a mOS of 7.5 (CI=5.9-11.3) months. The HR for the CD274 CN>specimen ploidy −1 group was 0.9 ([0.7-1.1], p=0.3). FIG. 8B shows the OS for patients with a CD274 CN<specimen ploidy −2 compared to patients with a CD274 CN>specimen ploidy −2. The mOS of patients with a CD274 CN>specimen ploidy −2 (N=548) was 9.3 (CI=7.5-11.5) months, while patients with a CD274 CN<specimen ploidy −2 (N=73) had a mOS of 6.7 (CI=4.9-14.2) months. The HR for the CD274 CN>specimen ploidy −2 group was 0.96 (CI=0.7-1.3; p=0.8). FIG. 8C shows the OS for patients with a CD274 CN<specimen ploidy −3 compared to patients with a CD274 CN>specimen ploidy −3. The mOS of patients with a CD274 CN>specimen ploidy −3 (N=614) was 8.9 (CI=7.4-11.2) months, while patients with a CD274 CN<specimen ploidy −3 (N=7) had a mOS of 2.3 (CI=0.4-NA) months. The HR for the CD274 CN>specimen ploidy −3 group was 1.3 (CI=0.5-3.5; p=0.6).

FIG. 9 depicts an exemplary device, in accordance with some embodiments.

FIG. 10 depicts an exemplary system, in accordance with some embodiments.

FIG. 11 depicts a block diagram of an exemplary process for detecting a CD274 copy number gain and a high TMB, in accordance with some embodiments.

FIG. 12 provides a non-limiting example of a process flowchart for detecting and calling CD274 copy number (CN) changes according to one instance of the disclosed methods.

### DETAILED DESCRIPTION

The present disclosure relates generally to detection of cluster of differentiation 274 (CD274) copy number alterations and/or tumor mutational burden (TMB) in cancer, as well as methods of treatment, and uses related thereto.

The present disclosure describes the results of comprehensive genomic profiling (CGP) of cancer patients treated with immunotherapy (e.g., immune checkpoint inhibitors). These analyses showed that, unexpectedly, a synergistic effect existed between CD274 gene copy number gains and high TMB in cancer (e.g., non-squamous non-small cell lung cancer) on responses to such treatment. For example, patients with a CD274 gene copy number greater than or equal to specimen ploidy +2 and a high TMB (of at least about 10 mutations/Megabase) had a median overall survival (mOS) of about 24.9 months, which was unexpectedly longer when compared to patients without both a CD274 gene copy number gain (e.g., a CD274 gene copy number greater than or equal to specimen ploidy +2) and a high TMB. See, e.g., Example 1. Accordingly, without wishing to be bound by theory, it is thought that the presence of a CD274 gene copy number gain and a high TMB in a cancer (e.g., in one or more samples from an individual having cancer) may identify cancer patients who are likely to respond to treatment with immunotherapies, such as immune checkpoint inhibitors, e.g., as described herein.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

As used herein, the terms “cluster of differentiation 274” or “CD274” refer to a gene encoding a programmed death-ligand 1 (PD-L1) mRNA or a PD-L1 polypeptide. CD274 is a gene located on chromosome 9p24.1. CD274 is also known as B7-H, B7-H1, B7H1, PD-L1, PDCD1LG1, PDL1. In some embodiments, a CD274 gene is a human CD274 gene.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2-O-methyl-, 2-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(O)R, P(0)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

The term “detection” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy including an immunotherapy, such as a checkpoint inhibitor). In some embodiments, a biomarker is a collection of genes and/or a collective number of mutations/alterations (e.g., somatic mutations) in a collection of genes, for example, a biomarker may comprise a CD274 copy number change (e.g., a CD274 copy number gain) and/or tumor mutational burden status (e.g., high tumor mutational burden). Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

The term “segmentation” (or “sequence segmentation”), as used herein, refers to a process for partitioning of sequence read data into a number of non-overlapping segments that cover all sequence read data points, such that each segment of a plurality of segments is as homogeneous as possible and all sequence reads associated with a given segment have the same copy number. In some instances, segmentation may be performed by processing aligned sequence read data (or other sequencing-related data, e.g., coverage data, allele frequency data, etc., derived from the sequence read data) using any of a variety of methods known to those of skill in the art (see., e.g., Braun and Miller (1998), “Statistical methods for DNA sequence segmentation”, Statistical Science 13(2):142-162). Examples of segmentation methods include, but are not limited to, circular binary segmentation (CBS) methods, maximum likelihood methods, hidden Markov chain methods, walking Markov methods, Bayesian methods, long-range correlation methods, change point methods, or any combination thereof.

A “reference sample,” “reference cell,” “reference tissue,” “control sample,” “control cell,” or “control tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent, and in some embodiments stop) cancer cell infiltration into peripheral organs; inhibit (i.e., slow to some extent, and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”, and the like) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient, individual or subject herein is a human.

As used herein, “administering” (and grammatical variations thereof such as “administration” or “administer”, and the like) refers to a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules, including, but not limited to, single or multiple administrations over various time-points, bolus administration, and pulse infusion are contemplated herein.

The terms “concurrently” or “in combination” are used herein to refer to administration of two or more therapeutic agents, where at least part of the administration overlaps in time. Accordingly, concurrent administration includes a dosing regimen wherein the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker described herein. In certain embodiments, the manufacture or kit is promoted, distributed, or sold as a unit for performing the methods described herein.

The phrase “based on” when used herein means that the information about one or more biomarkers is used to inform a treatment decision, information provided on a package insert, or marketing/promotional guidance, etc.

### III. Methods, Systems, and Devices

In some aspects, provided herein are methods for identifying an individual having a cancer who may benefit from a treatment comprising an immunotherapy. In other aspects, provided herein are methods for selecting a therapy or treatment for an individual having a cancer. In other aspects, provided herein are methods for identifying one or more treatment options for an individual having a cancer. In other aspects, provided herein are methods for predicting survival of an individual having a cancer. In other aspects, provided herein are methods for predicting survival of an individual having a cancer treated with a treatment comprising an immunotherapy. In other aspects, provided herein are methods for treating or delaying progression of cancer. In other aspects, provided herein are methods for monitoring, evaluating or screening an individual having a cancer. In other aspects, provided herein are methods for assessing a CD274 gene copy number alteration, such as a CD274 gene copy number gain, and/or a high tumor mutational burden in a cancer in an individual. In other aspects, provided herein are methods for detecting a CD274 gene copy number alteration, such as a CD274 gene copy number gain, and/or a high tumor mutational burden in one or more samples from an individual having a cancer. In other aspects, provided herein are methods for detecting the presence or absence of a cancer and a CD274 gene copy number alteration, such as a CD274 gene copy number gain, and/or a high tumor mutational burden in an individual. In other aspects, provided herein are methods for monitoring progression or recurrence of a cancer in an individual. In other aspects, provided herein are methods for identifying a candidate treatment for a cancer in an individual in need thereof.

In some embodiments of any of the methods provided herein, the methods comprise detecting the presence or absence of a CD274 gene copy number alteration, such as a CD274 gene copy number gain, and/or a high tumor mutational burden, in one or more samples from an individual. In other embodiments of any of the methods provided herein, the methods comprise acquiring knowledge of the presence or absence of a CD274 gene copy number alteration, such as a CD274 gene copy number gain, and/or a high tumor mutational burden in one or more samples from an individual. In some embodiments, detection of a CD274 gene copy number alteration, such as a CD274 gene copy number gain, and a high tumor mutational burden in the sample(s) identifies the individual as one who may benefit from the treatment comprising an immunotherapy. In some embodiments, the methods further comprise generating a report comprising one or more treatment options identified for the individual based at least in part on detection of the CD274 gene copy number alteration, such as a CD274 gene copy number gain, and high tumor mutational burden in the sample(s), wherein the one or more treatment options comprise an immunotherapy. In some embodiments, the methods further comprise generating a report comprising one or more treatment options identified for the individual based at least in part on knowledge of the presence of the CD274 gene copy number alteration, such as a CD274 gene copy number gain, and high tumor mutational burden, in the sample(s) from the individual, wherein the one or more treatment options comprise an immunotherapy. In some embodiments, responsive to the acquisition of knowledge of the presence of the CD274 gene copy number alteration, such as a CD274 gene copy number gain, and/or high tumor mutational burden in the sample(s) from the individual: (i) the individual is classified as a candidate to receive a treatment comprising an immunotherapy; and/or (ii) the individual is identified as likely to respond to a treatment that comprises an immunotherapy. In some embodiments, responsive to the acquisition of knowledge of the presence of the CD274 gene copy number alteration, such as a CD274 gene copy number gain, and/or high tumor mutational burden in the sample(s) from the individual, the individual is predicted to have longer survival when treated with a treatment comprising an immunotherapy, as compared to survival of an individual whose cancer does not comprise a CD274 gene copy number alteration, such as a CD274 gene copy number gain, and/or a high tumor mutational burden. In some embodiments, responsive to the acquisition of knowledge of the presence of the CD274 gene copy number alteration, such as a CD274 gene copy number gain, and/or high tumor mutational burden in the sample(s) from the individual, the method comprises administering to the individual an effective amount of a treatment that comprises an immunotherapy. In some embodiments, responsive to the acquisition of knowledge of the presence of the CD274 gene copy number alteration, such as a CD274 gene copy number gain, and/or high tumor mutational burden in the sample(s) from the individual, the individual is predicted to have decreased risk of cancer progression or cancer recurrence when treated with a treatment comprising an immunotherapy, as compared to an individual whose cancer does not comprise a CD274 gene copy number alteration, such as a CD274 gene copy number gain, and/or a high tumor mutational burden. In some embodiments, responsive to the acquisition of knowledge of the presence of the CD274 gene copy number alteration, such as a CD274 gene copy number gain, and/or high tumor mutational burden in the sample(s) from the individual, the individual is predicted to have an improved response to a treatment comprising an immunotherapy and/or longer survival when treated with a treatment comprising an immunotherapy, as compared to an individual whose cancer does not comprise a CD274 gene copy number alteration, such as a CD274 gene copy number gain, and/or a high tumor mutational burden. In some embodiments, the methods provided herein comprise providing an assessment of a CD274 gene copy number alteration, such as a CD274 gene copy number gain, and/or a high tumor mutational burden, e.g., in an individual or in one or more samples from an individual. In some embodiments, the methods provided herein comprise detecting the CD274 gene copy number alteration, such as a CD274 gene copy number gain, and/or high tumor mutational burden, in one or more samples from an individual, and administering to the individual an effective amount of a treatment that comprises an immunotherapy.

In other aspects, provided herein are systems and non-transitory computer readable storage media. In some embodiments, the systems and non-transitory computer readable storage media provided herein are for (e.g., are configured for) identifying an individual having a cancer who may benefit from a treatment comprising an immunotherapy. In some embodiments, the systems and non-transitory computer readable storage media provided herein are for (e.g., are configured for) detecting, in one or more samples from an individual having cancer, a CD274 gene copy number alteration, such as a CD274 gene copy number gain, and/or a high tumor mutational burden. In some embodiments, detecting the CD274 gene copy number alteration, such as a CD274 gene copy number gain, and high tumor mutational burden in the sample(s) from the individual identifies the individual as one who may benefit from a treatment comprising an immunotherapy.

### A. CD274 Copy Number Alterations and Detection Methods

Certain aspects of the present disclosure relate to CD274 gene copy number alterations, such as CD274 gene copy number gains.

As demonstrated herein, CD274 gene copy number alterations, such as CD274 gene copy number gains, in cancer with high tumor mutational burden may be predictive of increased survival and/or increased likelihood of response when treated with an immunotherapy, such as an immune checkpoint inhibitor. See, e.g., Example 1. Accordingly, in some embodiments, provided herein are methods that comprise acquiring knowledge of or detecting a CD274 gene copy number alteration, such as a CD274 gene copy number gain, in a cancer. In some embodiments, the methods of the disclosure also comprise acquiring knowledge of or detecting a high tumor mutational burden in the cancer (see, e.g., Section B, below).

As used herein “cluster of differentiation 274” or “CD274” refer to a gene encoding a CD274 mRNA, or a Programmed death-ligand 1 (PD-L1) polypeptide. CD274 is also known as PD-L1, PDL1, B7-H1, B7-H, CD274 molecule, PDCD1L1, PDCD1LG1, cluster of differentiation 274, programmed death-ligand 1, and B7 homolog 1. In some embodiments, a CD274 gene is a human CD274 gene. An exemplary CD274 gene is represented by NCBI Gene ID No. 29126. In some embodiments, a CD274 gene is located at chromosomal coordinates chr9:5,450,542-5,470,554 according to human genome version hg19. An exemplary CD274 mRNA sequence is represented by NCBI Ref. Seq. NM_014143, provided below as SEQ ID NO: 1:

(SEQ ID NO: 1)
AGTTCTGCGCAGCTTCCCGAGGCTCCGCACCAGCCGCGCTTCTGT
CCGCCTGCAGGGCATTCCAGAAAGATGAGGATATTTGCTGTCTTT
ATATTCATGACCTACTGGCATTTGCTGAACGCATTTACTGTCACG
GTTCCCAAGGACCTATATGTGGTAGAGTATGGTAGCAATATGACA
ATTGAATGCAAATTCCCAGTAGAAAAACAATTAGACCTGGCTGCA
CTAATTGTCTATTGGGAAATGGAGGATAAGAACATTATTCAATTT
GTGCATGGAGAGGAAGACCTGAAGGTTCAGCATAGTAGCTACAGA
CAGAGGGCCCGGCTGTTGAAGGACCAGCTCTCCCTGGGAAATGCT
GCACTTCAGATCACAGATGTGAAATTGCAGGATGCAGGGGTGTAC
CGCTGCATGATCAGCTATGGTGGTGCCGACTACAAGCGAATTACT
GTGAAAGTCAATGCCCCATACAACAAAATCAACCAAAGAATTTTG
GTTGTGGATCCAGTCACCTCTGAACATGAACTGACATGTCAGGCT
GAGGGCTACCCCAAGGCCGAAGTCATCTGGACAAGCAGTGACCAT
CAAGTCCTGAGTGGTAAGACCACCACCACCAATTCCAAGAGAGAG
GAGAAGCTTTTCAATGTGACCAGCACACTGAGAATCAACACAACA
ACTAATGAGATTTTCTACTGCACTTTTAGGAGATTAGATCCTGAG
GAAAACCATACAGCTGAATTGGTCATCCCAGAACTACCTCTGGCA
CATCCTCCAAATGAAAGGACTCACTTGGTAATTCTGGGAGCCATC
TTATTATGCCTTGGTGTAGCACTGACATTCATCTTCCGTTTAAGA
AAAGGGAGAATGATGGATGTGAAAAAATGTGGCATCCAAGATACA
AACTCAAAGAAGCAAAGTGATACACATTTGGAGGAGACGTAATCC
AGCATTGGAACTTCTGATCTTCAAGCAGGGATTCTCAACCTGTGG
TTTAGGGGTTCATCGGGGCTGAGCGTGACAAGAGGAAGGAATGGG
CCCGTGGGATGCAGGCAATGTGGGACTTAAAAGGCCCAAGCACTG
AAAATGGAACCTGGCGAAAGCAGAGGAGGAGAATGAAGAAAGATG
GAGTCAAACAGGGAGCCTGGAGGGAGACCTTGATACTTTCAAATG
CCTGAGGGGCTCATCGACGCCTGTGACAGGGAGAAAGGATACTTC
TGAACAAGGAGCCTCCAAGCAAATCATCCATTGCTCATCCTAGGA
AGACGGGTTGAGAATCCCTAATTTGAGGGTCAGTTCCTGCAGAAG
TGCCCTTTGCCTCCACTCAATGCCTCAATTTGTTTTCTGCATGAC
TGAGAGTCTCAGTGTTGGAACGGGACAGTATTTATGTATGAGTTT
TTCCTATTTATTTTGAGTCTGTGAGGTCTTCTTGTCATGTGAGTG
TGGTTGTGAATGATTTCTTTTGAAGATATATTGTAGTAGATGTTA
CAATTTTGTCGCCAAACTAAACTTGCTGCTTAATGATTTGCTCAC
ATCTAGTAAAACATGGAGTATTTGTAAGGTGCTTGGTCTCCTCTA
TAACTACAAGTATACATTGGAAGCATAAAGATCAAACCGTTGGTT
GCATAGGATGTCACCTTTATTTAACCCATTAATACTCTGGTTGAC
CTAATCTTATTCTCAGACCTCAAGTGTCTGTGCAGTATCTGTTCC
ATTTAAATATCAGCTTTACAATTATGTGGTAGCCTACACACATAA
TCTCATTTCATCGCTGTAACCACCCTGTTGTGATAACCACTATTA
TTTTACCCATCGTACAGCTGAGGAAGCAAACAGATTAAGTAACTT
GCCCAAACCAGTAAATAGCAGACCTCAGACTGCCACCCACTGTCC
TTTTATAATACAATTTACAGCTATATTTTACTTTAAGCAATTCTT
TTATTCAAAAACCATTTATTAAGTGCCCTTGCAATATCAATCGCT
GTGCCAGGCATTGAATCTACAGATGTGAGCAAGACAAAGTACCTG
TCCTCAAGGAGCTCATAGTATAATGAGGAGATTAACAAGAAAATG
TATTATTACAATTTAGTCCAGTGTCATAGCATAAGGATGATGCGA
GGGGAAAACCCGAGCAGTGTTGCCAAGAGGAGGAAATAGGCCAAT
GTGGTCTGGGACGGTTGGATATACTTAAACATCTTAATAATCAGA
GTAATTTTCATTTACAAAGAGAGGTCGGTACTTAAAATAACCCTG
AAAAATAACACTGGAATTCCTTTTCTAGCATTATATTTATTCCTG
ATTTGCCTTTGCCATATAATCTAATGCTTGTTTATATAGTGTCTG
GTATTGTTTAACAGTTCTGTCTTTTCTATTTAAATGCCACTAAAT
TTTAAATTCATACCTTTCCATGATTCAAAATTCAAAAGATCCCAT
GGGAGATGGTTGGAAAATCTCCACTTCATCCTCCAAGCCATTCAA
GTTTCCTTTCCAGAAGCAACTGCTACTGCCTTTCATTCATATGTT
CTTCTAAAGATAGTCTACATTTGGAAATGTATGTTAAAAGCACGT
ATTTTTAAAATTTTTTTCCTAAATAGTAACACATTGTATGTCTGC
TGTGTACTTTGCTATTTTTATTTATTTTAGTGTTTCTTATATAGC
AGATGGAATGAATTTGAAGTTCCCAGGGCTGAGGATCCATGCCTT
CTTTGTTTCTAAGTTATCTTTCCCATAGCTTTTCATTATCTTTCA
TATGATCCAGTATATGTTAAATATGTCCTACATATACATTTAGAC
AACCACCATTTGTTAAGTATTTGCTCTAGGACAGAGTTTGGATTT
GTTTATGTTTGCTCAAAAGGAGACCCATGGGCTCTCCAGGGTGCA
CTGAGTCAATCTAGTCCTAAAAAGCAATCTTATTATTAACTCTGT
ATGACAGAATCATGTCTGGAACTTTTGTTTTCTGCTTTCTGTCAA
GTATAAACTTCACTTTGATGCTGTACTTGCAAAATCACATTTTCT
TTCTGGAAATTCCGGCAGTGTACCTTGACTGCTAGCTACCCTGTG
CCAGAAAAGCCTCATTCGTTGTGCTTGAACCCTTGAATGCCACCA
GCTGTCATCACTACACAGCCCTCCTAAGAGGCTTCCTGGAGGTTT
CGAGATTCAGATGCCCTGGGAGATCCCAGAGTTTCCTTTCCCTCT
TGGCCATATTCTGGTGTCAATGACAAGGAGTACCTTGGCTTTGCC
ACATGTCAAGGCTGAAGAAACAGTGTCTCCAACAGAGCTCCTTGT
GTTATCTGTTTGTACATGTGCATTTGTACAGTAATTGGTGTGACA
GTGTTCTTTGTGTGAATTACAGGCAAGAATTGTGGCTGAGCAAGG
CACATAGTCTACTCAGTCTATTCCTAAGTCCTAACTCCTCCTTGT
GGTGTTGGATTTGTAAGGCACTTTATCCCTTTTGTCTCATGTTTC
ATCGTAAATGGCATAGGCAGAGATGATACCTAATTCTGCATTTGA
TTGTCACTTTTTGTACCTGCATTAATTTAATAAAATATTCTTATT
TATTTTGTTACTTGGTACACCAGCATGTCCATTTTCTTGTTTATT
TTGTGTTTAATAAAATGTTCAGTTTAACATCCCA

An exemplary amino acid sequence of a PD-L1 polypeptide is represented by NCBI Ref. Seq. NP_054862, provided below as SEQ ID NO: 2:

(SEQ ID NO: 2)
MRIFAVFIFMTYWHLLNAFTVTVPKDLYVVEYGSNMTIECKFPVE
KQLDLAALIVYWEMEDKNIIQFVHGEEDLKVQHSSYRQRARLLKD
QLSLGNAALQITDVKLQDAGVYRCMISYGGADYKRITVKVNAPYN
KINQRILVVDPVTSEHELTCQAEGYPKAEVIWTSSDHQVLSGKTT
TTNSKREEKLFNVTSTLRINTTINEIFYCTFRRLDPEENHTAELV
IPELPLAHPPNERTHLVILGAILLCLGVALIFIFRLRKGRMMDVK
KCGIQDTNSKKQSDTHLEET

CD274 gene copy number alterations refer to a variation in the copy number of a CD274 gene (e.g., a gain or loss of one or more copies of a CD274 gene) in a tumor or cancerous cell, that results in a variation from the copy number of a CD274 gene in a normal cell, e.g., a variation from two copies of a CD274 gene in a normal human somatic cell. Thus, a CD274 copy number gain refers to a gain of one or more copies of a CD274 gene in a tumor or cancerous cell, that results in a variation from the copy number of a CD274 gene in a normal cell, e.g., a variation from two copies of a CD274 gene in a normal human somatic cell. In some embodiments, a CD274 copy number gain refers to a gain of at least one CD274 gene copy in a tumor or cancerous cell, as compared to two copies of a CD274 gene in a normal human somatic cell (i.e., a total of at least three copies of a CD274 gene in the tumor or cancerous cell, as compared to two copies of a CD274 gene in a normal human somatic cell). CD274 gene copy number alterations may occur due to structural variations in the genome, such as duplications or deletions of genomic segments, as well as other chromosomal rearrangements and variations.

CD274 gene copy number alterations, such as CD274 gene copy number gains, in cancer may be assessed using any suitable method known in the art. Exemplary and non-limiting methods for detecting CD274 gene copy number alterations, such as CD274 gene copy number gains, include fluorescence in situ hybridization (FISH), comprehensive genomic profiling (CGP), comparative genomic hybridization (CGH), sequencing, microarray based-methods, amplification-based methods, or any combination thereof. In some embodiments, CD274 gene copy number alterations, such as CD274 gene copy number gains, are detected in vitro. Certain methods for detecting CD274 gene copy number alterations, such as CD274 gene copy number gains, are described in further detail below as non-limiting examples.

### (i) FISH

In some embodiments, FISH analysis is used to assess CD274 gene copy number alterations, such as CD274 gene copy number gains, in a cancer (e.g., in a sample from a cancer). Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, CD274 gene copy number alterations, such as CD274 gene copy number gains, in a cancer (e.g., in a sample from a cancer) are assessed using FISH according to any suitable method known in the art, such as the methods described in Inoue et al., JAMA Network Open (2020) 3(9):e2011818.

In some embodiments, CD274 gene copy number alterations, such as CD274 gene copy number gains, in a cancer (e.g., in a sample from a cancer) are assessed using a FISH probe that binds to a CD274 gene. In some embodiments, the FISH probe that binds to a CD274 gene is detectably labeled, e.g. fluorescently labeled. In some embodiments, CD274 gene copy number alterations, such as CD274 gene copy number gains, are assessed based on the amount of signal, e.g., fluorescent signal, corresponding to the FISH probe that binds to a CD274 gene, e.g. during visualization. In some embodiments, CD274 gene copy number alterations, such as CD274 gene copy number gains, are assessed relative to a control FISH probe. In some embodiments, the control FISH probe binds to a control locus (e.g., a control gene, chromosome or chromosome segment, DNA segment, centromere or centromere segment, and the like). In some embodiments, the control FISH probe binds to centromeric DNA. In some embodiments, the control FISH probe is a centromere enumeration probe, e.g., corresponding to any of human chromosomes 1, 2, 3, 4, 5, 6, 7, 8, 9, 10, 11, 12, 13, 14, 15, 16, 17, 18, 19, 20, 21, 22, X, Y or any combination thereof. In some embodiments, the control FISH probe is a CEP1, CEP2, CEP3, CEP4, CEP5, CEP6, CEP7, CEP8, CEP9, CEP10, CEP11, CEP12, CEP13, CEP14, CEP15, CEP16, CEP17, CEP18, CEP19, CEP20, CEP21, CEP22, CEPX, or CEPY FISH probe, or any combination thereof. In some embodiments, the control FISH probe is a CEP9 probe. In some embodiments, the control FISH probe is detectably labeled, e.g. fluorescently labeled. In some embodiments, CD274 gene copy number alterations, such as CD274 gene copy number gains, are assessed based on the ratio of the amount of signal, e.g., fluorescent signal, corresponding to the FISH probe that binds to a CD274 gene, to the amount of signal, e.g., fluorescent signal, corresponding to the control probe, such as any control probe known in the art or described herein. In some embodiments, CD274 gene copy number alterations, such as CD274 gene copy number gains, are assessed based on the ratio of the amount of signal, e.g., fluorescent signal, corresponding to the FISH probe that binds to a CD274 gene, to the amount of signal, e.g., fluorescent signal, corresponding to a centromere enumeration probe for chromosome 9 (e.g., a CEP9 probe). In some embodiments, the CD274 gene copy number gain in the cancer results in a ratio of signal from the FISH probe that binds to CD274 to signal from the control FISH probe (e.g., a centromere enumeration probe for chromosome 9, such as a CEP9 probe) of any of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments, the CD274 gene copy number gain in the cancer results in a ratio of signal from the FISH probe that binds to CD274 to signal from the control FISH probe (e.g., a centromere enumeration probe for chromosome 9, such as a CEP9 probe) of at least 1.5. In some embodiments, the CD274 gene copy number gain in the cancer results in a ratio of signal from the FISH probe that binds to CD274 to signal from the control FISH probe (e.g., a centromere enumeration probe for chromosome 9, such as a CEP9 probe) of at least 2. In some embodiments, a ratio of signal from the FISH probe that binds to CD274 to signal from the control FISH probe (e.g., a centromere enumeration probe for chromosome 9, such as a CEP9 probe) of at least 2 corresponds to a CD274 gene copy number of at least 4 in a diploid cancer (e.g., in a diploid cancer or tumor sample).

### (ii) CGH

In some embodiments, CD274 gene copy number alterations, such as CD274 gene copy number gains, in a cancer (e.g., in a sample from a cancer) are assessed using an array-based method, such as array-based comparative genomic hybridization (CGH) methods.

In array-based CGH methods, a first sample of nucleic acids (e.g., from a sample, such as from a cancer, a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, e.g., which have been spotted on the array. In some embodiments, the microarray is a whole genome microarray, e.g., it comprises DNA fragments or oligonucleotides with nucleotide sequences that cover the entire genome. In some embodiments, the microarray is a targeted microarray, e.g., it comprises DNA fragments or oligonucleotides with nucleotide sequences that cover a particular segment of the genome, such as a particular chromosome or chromosomal segment, such as chromosome 9 or a segment of chromosome 9 including the CD274 locus. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. In some embodiments, where there are gene copy number alterations (e.g., a CD274 copy number alteration, such as a CD274 copy number gain), differences in the ratio of the signals from the two labels are detected and the ratio provides a measure of the gene copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a cancer, a tumor, or a tissue or liquid biopsy) is labeled and hybridized to a second array (with identical content) and absolute signals are read. Copy number differences are calculated based on absolute signals from the two arrays.

In some embodiments, CD274 gene copy number alterations, such as CD274 gene copy number gains, are assessed based on the ratio of the amount of signal from a first sample of nucleic acids from a cancer labeled with a first label, to the amount of signal from a second sample of control nucleic acids (e.g., such as from a healthy cell/tissue) labeled with a second label, wherein the ratio is assessed within CGH array(s) (e.g., as described above) at one or more positions comprising DNA fragments or oligonucleotides corresponding to chromosome 9 or portions thereof, or CD274 or portions thereof. In some embodiments, the ratio in a cancer having a CD274 gene copy number gain is any of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments, the ratio is at least 1.5. In some embodiments, the ratio is at least 2.

(iii) Amplification-Based Methods

In some embodiments, CD274 gene copy number alterations, such as CD274 gene copy number gains, in a cancer (e.g., in a sample from a cancer) are assessed using an amplification-based method, such as a PCR method, e.g. quantitative PCR (qPCR) or digital droplet PCR (ddPCR), and the like.

In some embodiments, CD274 gene copy number alterations, such as CD274 gene copy number gains, in a cancer (e.g., in a sample from a cancer) are assessed using a qPCR or ddPCR method. In qPCR, a test locus (e.g., CD274) and a reference locus (e.g., a control locus, e.g., such as a housekeeping gene, actin, tubulin, etc.) are amplified by polymerase chain reaction. The amount of target DNA amplified during the PCR cycles is quantified using probes that detect the PCR amplification product as it accumulates during PCR cycles. Such probes include, without limitation, SYBR Green, which is a double-stranded DNA binding dye, and TaqMan, which is fluorogenic probe that includes a sequence-specific oligonucleotide probe conjugated to a fluorescent reporter dye and a quencher dye. Copy number alterations (e.g., CD274 gene copy number alterations, such as CD274 gene copy number gains) may be assessed based on the amount of signal (e.g., fluorescence signal) from amplification of the test locus (e.g., CD274) relative to signal (e.g., fluorescence signal) from amplification of the reference locus. When the copy number of the reference locus is known, the absolute copy number of the test locus (e.g., CD274) may be determined based on the amount of signal from amplification of the test locus (e.g., CD274) relative to signal from amplification of the reference locus. In ddPCR, individual PCR reactions targeting a test locus (e.g., CD274) and a reference locus (e.g., a control locus, e.g., such as a housekeeping gene, actin, tubulin, etc.) are partitioned into fluid individual droplets. The PCR reactions in the individual droplets are allowed to amplify the test locus and reference locus, and the amount of amplified DNA is assessed by fluorescence signal for each droplet at the end of the PCR reactions. Copy number alterations (e.g., CD274 gene copy number alterations, such as CD274 gene copy number gains) are assessed by comparing the number of amplified DNA molecules arising from the test locus (e.g., CD274) to the number of amplified DNA molecules arising from the reference locus. In some embodiments, the ratio of the relative gene copy numbers of the test locus (e.g., CD274) and reference locus (e.g., a control locus) assessed in a sample from a cancer having a CD274 gene copy number gain is any of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments, the ratio is at least 1.5. In some embodiments, the ratio is at least 2. See, e.g., Ma and Chung, Curr Protoc Hum Genet. 2014 Jan. 21; 80: 7.21.1-7.21.8; and Bell et al., Methods Mol Biol. 2018; 1768:143-160.

### (iv) Sequencing

In some embodiments, CD274 gene copy number alterations, such as CD274 gene copy number gains, in a cancer (e.g., in a sample from a cancer) are assessed using a sequencing method. Any method of sequencing known in the art may be used to detect CD274 gene copy number alterations, such as CD274 gene copy number gains. Exemplary sequencing methods that may be used include those based on techniques developed by Maxam and Gilbert or Sanger. Automated sequencing procedures may also be used, e.g., including sequencing by mass spectrometry. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next-generation sequencing (NGS). In some embodiments, the sequencing comprises hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference.

Next-generation sequencing includes any sequencing method that determines the nucleotide sequence of either individual nucleic acid molecules or clonally expanded proxies for individual nucleic acid molecules in a highly parallel fashion (e.g., greater than 105 molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect CD274 gene copy number alterations, such as CD274 gene copy number gains, in a cancer (e.g., in a sample from a cancer) include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect CD274 gene copy number alterations, such as CD274 gene copy number gains, in a cancer (e.g., in a sample from a cancer) include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments, methods for detecting CD274 gene copy number alterations, such as CD274 gene copy number gains, in a cancer (e.g., in a sample from a cancer) comprise providing a sample from an individual (e.g., an individual having cancer), wherein the sample comprises one or more nucleic acids. In some embodiments, the methods further comprise preparing a nucleic acid sequencing library from the one or more nucleic acids in the sample. Methods for the preparation of nucleic acid sequencing libraries, e.g., suitable for any of the sequencing methods described herein (e.g., NGS and/or hybrid-capture NGS), are known in the art. In some embodiments, the sequencing library is prepared as described in Frampton et al., (2013) Nat Biotechnol, 31:1023-1031. In an exemplary method, nucleic acids, e.g., double stranded DNA (dsDNA), are fragmented, for example, using sonication. In some embodiments, nucleic acids are fragmented to a length of about 200 base pairs. In some embodiments, the fragmented nucleic acids are purified, e.g., using any suitable method, such as using AMPure XP Beads (Agencourt) and/or solid phase reversible immobilization (SPRI) methods. In some embodiments, sequencing library construction using the purified nucleic acids is carried out using any suitable method, e.g., using commercially available library preparation kits, such as an NEBNext kit (e.g., available from New England Biolabs). In some embodiments, library preparation is performed using a “with-bead” protocol. See, e.g., Fisher et al., Genome Biol (2011) 12:R1. In some embodiments, the library preparation method is selected based on the sequencing method used, e.g., an NEBNext kit is suitable for use with NGS sequencing platforms from Illumina Inc. In some embodiments, a sequencing library indexed, e.g., with barcodes such as six base pair barcodes, is amplified, e.g., using any suitable method, such as PCR. In some embodiments, amplified nucleic acids are purified using any suitable method, such as SPRI purification. In some embodiments, the methods further comprise quantifying the amplified and/or purified nucleic acids, e.g., by qPCR. In some embodiments, the methods further comprise sizing the amplified and/or purified nucleic acids using any suitable method, such as using a LabChip GX system, e.g., available from Caliper Life Sciences. In some embodiments, size selection is not performed.

In some embodiments, the methods further comprise selectively enriching for one or more nucleic acids (e.g., one or more nucleic acids corresponding to CD274) to produce an enriched sample. In some embodiments, the selectively enriching is performed on a sequencing library, e.g., a sequencing library prepared according to the methods described herein. In some embodiments, the selectively enriching is performed as described in Frampton et al., (2013) Nat Biotechnol, 31:1023-1031. In an exemplary process, the methods comprise performing solution hybridization using 5′-biotinylated DNA oligonucleotide baits, which may be prepared or synthesized using any suitable method known in the art, e.g., as described in Frampton et al., (2013) Nat Biotechnol, 31:1023-1031. In some embodiments, the methods comprise denaturing the sequencing library. In some embodiments, denaturing is performed at a temperature of about 95° C., e.g., for about 5 minutes. In some embodiments, the methods further comprise incubating the denatured sequencing library at a temperature of about 68° C., e.g., for about 5 minutes. In some embodiments, the methods further comprise mixing the sequencing library with baits, and optionally Cot, salmon sperm, and/or adaptor-specific blocker DNA in hybridization buffer. In some embodiments, the mixture is incubated for about 24 hours. In some embodiments, the methods further comprise capturing sequencing library-bait duplexes using any suitable method, such as using paramagnetic MyOne streptavidin beads (available from Invitrogen). In some embodiments, the methods further comprise washing to remove off-target library. In some embodiments, the methods further comprise amplifying the captured sequencing library, e.g., using PCR. In some embodiments, the methods further comprise purifying the amplification products using any suitable method, such as SPRI purification. In some embodiments, the methods further comprise quantifying the amplified and/or purified nucleic acids, e.g., by qPCR or any other suitable method. In some embodiments, the methods further comprise sizing the amplified and/or purified nucleic acids using any suitable method, such as using a LabChip GX system, e.g., available from Caliper Life Sciences. In some embodiments, the methods further comprise sequencing using any suitable method or system known in the art, e.g., as described herein. In some embodiments, sequencing is performed using a next-generation sequencer, such as an Illumina HiSeq 2000 system. In some embodiments, sequencing is performed using paired-end sequencing. In some embodiments, the sequencing is performed as described in Frampton et al., (2013) Nat Biotechnol, 31:1023-1031.

In some embodiments, the methods further comprise analyzing sequence data obtained from the sequencing, e.g., a plurality of sequence reads, for the presence or absence of CD274 gene copy number alterations, such as CD274 gene copy number gains. In some embodiments, the analysis is performed as described in Frampton et al., (2013) Nat Biotechnol, 31:1023-1031, and/or Sun et al., PLoS Comput Biol. 2018 Feb. 7; 14(2):e1005965. In some embodiments, analyzing sequence data, e.g., a plurality of sequence reads, for the presence or absence of CD274 gene copy number alterations, such as CD274 gene copy number gains, comprises one or more, or all, of the steps as described in Frampton et al., (2013) Nat Biotechnol, 31:1023-1031 for assessing copy number alterations. In some embodiments, analyzing sequence data, e.g., a plurality of sequence reads, for the presence or absence of CD274 gene copy number alterations, such as CD274 gene copy number gains, comprises one or more, or all, of the steps as described in Sun et al., PLoS Comput Biol. 2018 Feb. 7; 14(2):e1005965 for assessing copy number alterations. In some embodiments, the analysis comprises one or more, or all, of the steps of: (a) generating a copy number model based on sequence data (e.g., a plurality of sequence reads); and (b) determining a CD274 gene copy number based on the copy number model. In some embodiments, the copy number model is generated according to any suitable method known in the art or described herein. Examples of copy number modeling methods include, but are not limited to sliding window methods for computing read count in non-overlapping windows, normalized depth-of-coverage and B allele frequency (i.e., the normalized measure of a relative signal intensity ratio for two alleles) methods, circularized binary segmentation (CBS) methods, statistical analyses of mapping density based on mean-shift approaches, hidden Markov models, read depth-based Bayesian information criteria methods, or any combination thereof (see, e.g., L1 and Olivier (2013), “Current analysis platforms and methods for detecting copy number variation”, Physiol. Genomics 45(1):1-16). In some embodiments, the copy number model is a genome-wide copy number model. In some embodiments, the copy number model is generated by: (a) aligning the sequence data (e.g., the plurality of sequence reads) against a reference genome; (b) normalizing sequence coverage distribution of the aligned sequence data (e.g., the aligned plurality of sequence reads) against a control sample (e.g., a paired normal control, a process-matched control, or a “panel of normal” control); and (c) generating segmentation data for the normalized sequence data (e.g., the normalized plurality of sequence reads). In some embodiments, the copy number model is generated by: (a) aligning the sequence data (e.g., the plurality of sequence reads) against a reference genome; (b) normalizing sequence coverage distribution of the aligned sequence data (e.g., the aligned plurality of sequence reads) against a process-matched control; and (c) performing segmentation of the normalized sequence data (e.g., the normalized plurality of sequence reads) into genomic segments of equal copy number. In some embodiments, the aligning is performed using any suitable method, such as using a BWA aligner (e.g., v0.5.9). See, e.g., L1 and Durbin, Bioinformatics (2010) 26:589-595. In some embodiments, the reference genome is a human genome, such as human genome version hg19, or any other suitable version or build. In some embodiments, PCR duplicate reads are removed, and/or sequence data metrics are collected using any suitable method, such as using Picard 1.47 (see, e.g., picard.sourceforge.net and/or broadinstitute.github.io/picard/) and/or Samtools 0.1.12a (see, e.g., L1 et al., Bioinformatics (2009) 25:2078-2079). In some embodiments, the method further comprises performing local alignment optimization using any suitable method, e.g., using GATK 1.0.4705 (see, e.g., DePristo et al., Nat Genet (2011) 43:491-498). In some embodiments, the normalizing comprises generating log-ratios of normalized coverage data for exonic, intronic, and single nucleotide polymorphisms (SNPs), and minor allele frequency (MAF) data based on the process-matched control. In some embodiments, the normalizing comprises dividing read depth of aligned sequence data (e.g., the aligned plurality of sequence reads) by read depth of the process-matched control, optionally followed by GC-content bias correction (e.g., using Lowess regression), to generate log-ratios. In some embodiments, the normalizing comprises generating minor allele frequency (MAF) data based on heterozygous genome-wide single nucleotide polymorphisms (SNPs). In some embodiments, the normalizing comprises accounting for stromal admixture. In some embodiments, the control sample is a process-matched control. In some embodiments, the process-matched control comprises one or more heterozygous diploid samples (e.g., a mixture of sequence data, such as sequence reads, from one or more heterozygous diploid samples). In some embodiments, the process-matched control comprises a mixture of any of about 2, about 3, about 4, about 5, about 6, about 7, about 8, about 9, about 10, about 11, about 12, about 13, about 14, about 15, about 16, about 17, about 18, about 19, about 20, or more, heterozygous diploid samples (e.g., a mixture of sequence data, such as sequence reads, from any of about 2, about 3, about 4, about 5, about 6, about 7, about 8, about 9, about 10, about 11, about 12, about 13, about 14, about 15, about 16, about 17, about 18, about 19, about 20, or more, heterozygous diploid samples). In some embodiments, the process-matched control is obtained from the HapMap project. In some embodiments, the segmentation is performed using any suitable method known in the art, such as a maximum likelihood method, a hidden Markov chain method, a walking Markov method, a Bayesian methods, a long-range correlation method, a change point method, a circular binary segmentation (CBS) method (see, e.g., Sun et al., PLoS Comput Biol. 2018 Feb. 7; 14(2):e1005965; and Olshen et al., Biostatistics. 2004; 5(4):557-72), or any combination thereof. In some embodiments, the segmentation comprises whole-genome segmentation. In some embodiments, the segmentation is performed on the log-ratio and/or MAF data as described above. In some embodiments, the method further comprises fitting the segmented data (e.g., the segmented log-ratio and/or MAF data) to one or more copy number models, such as a Gibbs-based or a grid-based copy number model to produce copy number estimates (e.g., genome-wide copy number estimates; see, e.g., Sun et al., PLoS Comput Biol. 2018 Feb. 7; 14(2):e1005965). In some embodiments, the method further comprises selecting by an automated heuristic the optimal copy number model. In some embodiments, the CD274 gene copy number is determined based on a summary statistic (e.g., mean, median, mode, minimum value, maximum value, range, or standard deviation) of the copy number of all segments of the segmented copy number model overlapping a CD274 gene. In some embodiments, the analyzing comprises accounting for sample, specimen, cancer or tumor purity and/or ploidy. See, e.g., Sun et al., PLoS Comput Biol. 2018 Feb. 7; 14(2):e1005965 for additional information about methods for assessing CD274 gene copy number alterations, such as CD274 gene copy number gains, that may be used in the methods of the disclosure. In some embodiments, sample, specimen, cancer or tumor ploidy or purity is estimated as described in Sun et al., PLoS Comput Biol. 2018 Feb. 7; 14(2):e1005965.

In some embodiments, analyzing sequence data, e.g., a plurality of sequence reads, for the presence or absence of CD274 gene copy number alterations, such as CD274 gene copy number gains, comprises one or more, or all, of: (i) a coverage normalization procedure, (ii) segmentation, (iii) an iterative sample contamination detection method, (iv) a copy number model determination method, and/or (v) calling of CD274 gene copy number alterations, such as CD274 gene copy number gains. In some embodiments, the coverage normalization procedure comprises using a “panel of normal.” In some embodiments, the coverage normalization procedure using a “panel of normal” provides proper normalization of chromosome X sequence read data that takes gender into account. In some embodiments, the segmentation is based on a pruned exact linear time (PELT) method customized to use a particular transformation of the coverage ratio data and extended to account for sample contamination. In some embodiments, the iterative sample contamination detection method is based on aberrant SNP profiles, e.g., determined using a base-substitution noise model and a copy number model profile to identify a contamination signal. In some embodiments, the copy number model determination method is based on determination of all locally optimal copy number model configurations and prioritization of models (e.g., the copy number model(s) that are most consistent with the sequence read data and are biologically plausible). In some embodiments, the calling of CD274 gene copy number alterations, such as CD274 gene copy number gains, comprises automated calling based both on the specific copy number model(s) and a scan for additional alterations not explicitly included in the overall copy number model.

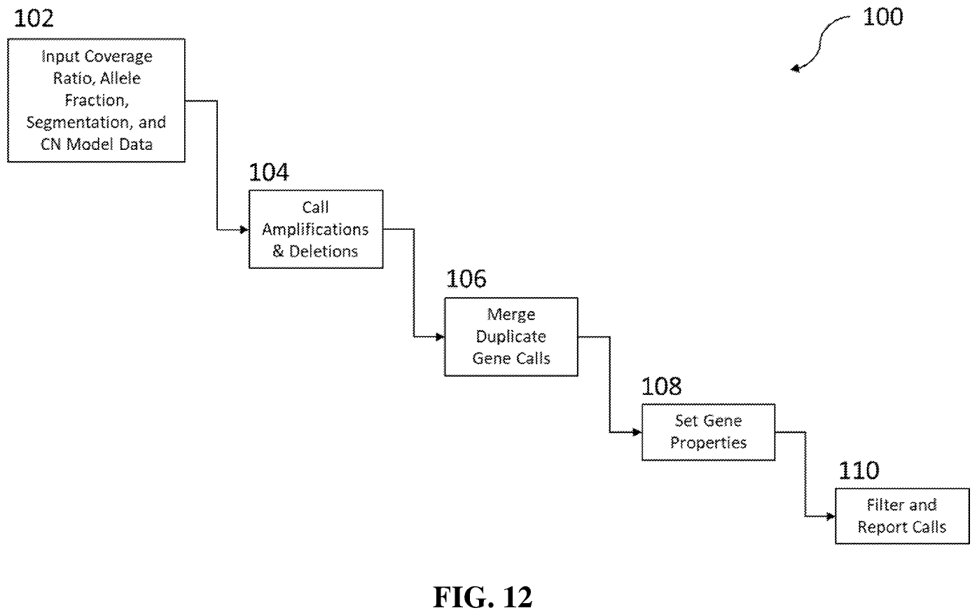

FIG. 12 provides a non-limiting example of a process flowchart (process 100) for analyzing sequence data, e.g., a plurality of sequence reads, for the presence or absence of CD274 gene copy number alterations, such as CD274 gene copy number gains. The described methods and systems utilize: (i) a coverage normalization procedure using a “panel of normal” approach that provides proper normalization of chromosome X sequence read data that takes gender into account, (ii) segmentation based on, e.g., a pruned exact linear time (PELT) method customized to use a particular transformation of the coverage ratio data and extended to account for sample contamination, (iii) an iterative sample contamination detection method based on aberrant SNP profiles (determined using a base-substitution noise model and a copy number model profile to identify a contamination signal), (iv) a copy number model determination method based on determination of all locally optimal copy number model configurations and prioritization of models (e.g., the copy number model(s) that are most consistent with the sequence read data and are biologically plausible), and/or (v) automated calling of copy number alterations based both on the specific copy number model(s) and a scan for additional alterations not explicitly included in the overall copy number model. As illustrated in FIG. 12, process 100 begins in step 102 with the input of sequencing coverage ratio data (or “coverage ratio data”), allele fraction data, segmentation data, and copy number model data derived by pre-processing of sequence read data for a plurality of sequence reads that overlap one or more gene loci (e.g., including CD274) within one or more subgenomic intervals in the sample to be analyzed. In some instances, the coverage ratio data for the sample is determined by aligning a plurality of sequence reads that overlap one or more gene loci (e.g., including CD274) within one or more subgenomic intervals in the sample and in a control (e.g., a paired normal control, a process-matched control, or a “panel of normal” control) to a reference genome, and determining a number of sequence reads that overlap each of the one or more gene loci (e.g., including CD274) within the one or more subgenomic intervals in the sample and in the control in order to normalize the coverage for the sample to that in the control. In some instances, e.g., if a paired normal control sample is not available, a process-matched control (e.g., a mixture of DNA from a plurality of HapMap cell lines) may be used instead of the paired normal control to normalize coverage. In some instances, e.g., if a paired normal control sample is not available, a “panel of normal” control may be used instead of the paired normal control to normalize coverage.

In some instances, a “panel of normal” (PoN) or “Tangent normalization” control method may be used to normalize sequencing coverage (see, e.g., Tabak, et al. (2019) “The Tangent copy-number inference pipeline for cancer genome analyses”, www.biorxiv.org/content/10.1101/566505v1.full.pdf). The Tangent normalization method is a method of normalizing tumor data in order to deal with noise in the data. Specifically, the Tangent method deals with reducing systemic noise resulting from differences in the experimental conditions under which sequencing data from tumors and/or their normal controls were generated. It has been shown that the Tangent normalization method yields a greater reduction in noise than conventional normalization methods.

To begin, let n_{N} be the number of normal non-patient samples (i.e., samples obtained from a plurality of healthy individuals) and n_{T} be the number of tumor samples. Let i be an element of the set [1, 2, . . . , n_{N}] and j be an element of the set [1, 2, . . . , n_{T}]. Define Nᵢ to be the vector of log 2 copy-ratio intensities in genomic order for the i^{th} normal sample. Similarly, define Tⱼ to be the vector of log 2 copy-ratio intensities in genomic order for the j^{th} tumor sample. The normal sample vectors and the tumor sample vectors are elements of the M-dimensional vector space of all possible coverage profiles. Now define a reference subspace N of the vector space of all possible coverage profiles to be the space that contains all linear combinations of the vectors {N₁, N₂, . . . , N_{nN}} of normal samples. N is called the “noise space” and is an (n_{N}−1)-dimensional plane.

Given this setup, the Tangent normalization method proceeds as follows. Start by determining, for each tumor sample vector Tⱼ, the vector in the noise space N that is closest to Tⱼ using a Euclidean metric. Denote this vector p(Tⱼ), the projection of Tⱼ onto N. p(Tⱼ) represents the profile of a normal sample characterized under similar conditions to Tⱼ. The normalization of Tⱼ can now be computed by calculating the difference between Tⱼ and the projection p(Tⱼ) of Tⱼ onto N: Normalization of Tⱼ=Tⱼ−p(Tⱼ)
The projection p(Tⱼ) can be computed directly using standard linear algebra techniques.

The PoN method uses observed patterns of systemic noise in the normal samples to remove typical variation. Chromosome X (chrX) has a specific pattern of half the coverage for gene loci on chrX in males since normal males only have one X chromosome. The PoN method thus removes this variation.

In some embodiments, the allele fraction data for the sample is determined by aligning a plurality of sequence reads that overlap one or more gene loci (e.g., including CD274) within one or more subgenomic intervals in the sample to a reference genome, detecting a number of different alleles present at the one or more gene loci (e.g., including CD274) in the one or more subgenomic intervals in the sample, and determining an allele fraction for the different alleles present at the one or more gene loci (e.g., including CD274) by dividing the number of sequence reads identified for a given allele sequence by the total number of sequence reads identified for the gene locus.

In some embodiments, the segmentation data for the sample is generated by aligning a plurality of sequence reads that overlap one or more gene loci (e.g., including CD274) within one or more subgenomic intervals in the sample to a reference genome, and processing the aligned sequence read data (or other sequencing-related data, e.g., coverage ratio data, allele frequency data, etc., derived from the sequence read data) using a segmentation algorithm (e.g., a circular binary segmentation (CBS) method, a maximum likelihood method, a hidden Markov chain method, a walking Markov method, a Bayesian methods, a long-range correlation method, a change point method, or any combination thereof) to generate a plurality of non-overlapping segments such that the sequence associated with a given segment have the same copy number.

In some embodiments, segmentation may be performed as part of a copy number modeling process to determine a copy number model that best accounts for the coverage ratio and allele fraction data. For example, in some instances, a copy number model may comprise: a purity estimate (e.g., a fraction of cells in the sample that were derived from a tumor), a segmentation (e.g., a division of the genome into components that have undergone either amplifications or losses), and an assignment of copy number state to each segment, wherein the copy number state is the number of genomic copies of that segment. In some instances, copy number modeling may be facilitated by transforming haploid coverage ratio data (for example, R_{A} and R_{B}, where R_{A} and R_{B} are the haploid coverage ratios for minor and major alleles A and B, respectively) into sum coverage ratio (R_{A}+R_{B}=(2+(C_{A}+C_{B}/)g)/(1+λg), where C_{A} and C_{B} are the allele counts for the minor and major alleles, A and B, respectively; g=ρ/(1−φ where p is the purity; where λ=(Ψ/2), and where Ψ is the ploidy) and difference coverage ratio (R_{A}−R_{B}=((C_{A}−C_{B})g)/(1+λg)) data for major and minor alleles, and plotting the difference coverage ratio data versus the sum coverage ratio data in a plot that may be overlaid with the segment data and a grid representing allowed copy number states.

In some embodiments, segmentation may be performed in an iterative manner while simultaneously detecting and correcting for sample contamination in the sequence read data. For example, in some instances, the method may comprise estimating a degree of contamination for the sample based on a distribution of minor allele frequencies for a selected set of heterozygous single nucleotide polymorphisms (SNPs). Then, using the estimated degree of contamination as an initial value for a minor allele frequency (MAF) threshold, the sequencing data is iteratively segmented while simultaneously excluding sequencing data from the segmentation process that comprises SNPs having minor allele frequencies that are below the MAF threshold. At each iteration, the remaining SNPs are classified as aberrant (i.e., likely due to contamination) if they have a minor allele frequency that is different from the MAF for other SNPs detected on the same segment, and the MAF threshold is incrementally adjusted based a comparison of the distribution of aberrant SNP minor allele frequencies to the expected distribution of minor allele frequencies for the selected set of heterozygous SNPs. The segmenting, classifying, and MAF threshold adjusting steps are repeated each time the MAF threshold is increased. When no further increase of the MAF threshold is required (or there is no further change in the distribution of aberrant SNP minor allele frequencies, or a specified maximum number of iterations has been reach), the segmentation data and an estimated degree of contamination for the sample (equal to the final value of the MAF threshold) is output. In some instances, the method further comprises using the segmentation data and estimated degree of contamination to build a copy number model that predicts a copy number for one or more gene loci.

In some embodiments, the segmentation data for the sample may be generated using a pruned exact linear time (PELT) method to determine a number of segments required to properly account for the aligned sequence read data (or other sequencing-related data, e.g., coverage ratio data, allele frequency data, etc., derived from the sequence read data), where each segment (and the sequence reads associated with the segment) has the same copy number. In some instances, the segmentation data is generated using a pruned exact linear time (PELT) method that has been customized to use a particular transformation of the coverage ratio and allele fraction data (e.g., a transformation that enables presentation of the coverage ratio and allele fraction data on the same graph while simultaneously overlaying the predicted copy-number states) and extended to account for sample contamination.

In some embodiments, a copy number model may be used to identify (or predict) the number of copies of each gene locus, the segmentation of the sample, the sample purity, and the sample ploidy (e.g., an average copy number for the sample) that best account for the measured coverage ratio and allele fraction data for the one or more gene loci (e.g., including CD274). In some instances, the input data used to generate the copy number model also includes coverage ratio and allele fraction data for single nucleotide polymorphisms (SNPs) and introns. The coverage ratio data is often transformed to log 2 coverage ratio data. Examples of copy number modeling methods include, but are not limited to sliding window methods for computing read count in non-overlapping windows, normalized depth-of-coverage and B allele frequency (i.e., the normalized measure of a relative signal intensity ratio for two alleles) methods, circularized binary segmentation (CBS) methods, statistical analyses of mapping density based on mean-shift approaches, hidden Markov models, read depth-based Bayesian information criteria methods, or any combination thereof (see, e.g., L1 and Olivier (2013), “Current analysis platforms and methods for detecting copy number variation”, Physiol. Genomics 45(1):1-16).

In some embodiments, the input coverage ratio data or copy number estimates used to generate the copy number model are rounded off to integer values. In some instances the output values reported by the finalized copy number model (e.g., predicted copy number values for segments) are integer values. In some instances, the output values reported by the finalized copy number model (e.g., sample purity, sample ploidy, and copy number values predicted for specific gene loci) are real numbers (i.e., continuous). In some instances, sub-clonal events (e.g., sub-clonal deletion events) may occur which do not fit an integer copy number values and may thus have non-integer predicted copy number values.

In some instances, a copy number model may determine that the sample purity (or tumor fraction) has a value ranging from 0.05 to 1.0. In some instances, the determined sample purity may be at least 0.05, at least 0.1, at least 0.2, at least 0.3, at least 0.4, at least 0.5, at least 0.6, at least 0.7, at least 0.8, at least 0.9, at least 0.95, at least 0.98, or at least 0.99. In some instances, the determined sample purity may be at most 0.99, at most 0.98, at most 0.95, at most 0.9, at most 0.8, at most 0.7, at most 0.6, at most 0.5, at most 0.4, at most 0.3, at most 0.2, at most 0.1, or at most 0.05. Any of the lower and upper values described in this paragraph may be combined to form a range included within the present disclosure, for example, in some instances, the determined sample purity may range from 0.1 to 0.8. Those of skill in the art will recognize that the determined sample purity in a given instance may have any value within this range, e.g., about 0.64.

In some instances, a copy number model may determine that the sample ploidy has a value ranging from 1.0 to 10.0. In some instances, the determined sample ploidy may be at least 1.0, at least 2.0, at least 3.0, at least 4.0, at least 5.0, at least 6.0, at least 7.0, at least 8.0, at least 9.0, or at least 10.0. In some instances, the determined sample ploidy may be at most 10.0, at most 9.0, at most 8.0, at most 7.0, at most 6.0, at most 5.0, at most 4.0, at most 3.0, at most 2.0, or at most 1.0. Any of the lower and upper values described in this paragraph may be combined to form a range included within the present disclosure, for example, in some instances, the determined sample ploidy may range from 1.0 to 8.0. Those of skill in the art will recognize that the determined sample ploidy in a given instance may have any value within this range, e.g., about 3.4. In some instances, the sample ploidy may be rounded off and reported as an integer value.

In some instances, a copy number model may predict a copy number for a given gene locus (or segment with which it is associated) ranging from 0 to 500. In some instances, the predicted copy number is at least 0, at least 2, at least 4, at least 6, at least 8, at least 10, at least 20, at least 40, at least 60, at least 80, at least 100, at least 200, at least 300, at least 400, or at least 500. In some instances, the predicted copy number is at most 500, at most 400, at most 300, at most 200, at most 100, at most 80, at most 60, at most 40, at most 20, at most 10, at most 8, at most 6, at most 4, at most 2, or at most 0. Any of the lower and upper values described in this paragraph may be combined to form a range included within the present disclosure, for example, in some instances, the predicted copy number may range from 1 to 100. Those of skill in the art will recognize that the predicted copy number may have any value within this range, e.g., 7. In some instances, the predicted copy number for a gene locus may be a real value number rather than an integer.

Referring again to FIG. 12, at step 104, amplifications or gain (e.g., increases in the number of copies of a gene locus) or deletions (e.g., deletions of a complete or partial gene locus) of each gene locus of the one or more loci being analyzed are determined on a segment by segment basis. For example, CD274 gene copy number alterations, such as CD274 gene copy number gains or losses are determined on a segment by segment basis.

At step 106 of FIG. 12, duplicate gene calls, or more formally, duplicate calls for “gene objects” (i.e., digital data constructs that hold a set of properties (e.g., sequence location, target allele sequences, coverage ratios, etc.) associated with a given gene locus) are merged. Duplicate calls may arise, for example, if a gene sequence is broken into two sub-sequences, and both sub-sequences are called as gene loci comprising amplifications/gains or deletions, thus generating more than one gene object for the locus. In other instances, deletions may be called using both a copy number prediction that comes directly from the copy-number model data and by a partial deletion scanning method (e.g., a method that looks for sequence read(s) that overlap but deviate significantly from the target allele sequence(s) and results in a partial deletion call), in which case more than one gene object is again generated for the locus. Upon merging, two or more gene objects and their corresponding properties (e.g., sequence location, target allele sequences, coverage ratios, etc.) will be replaced by a single gene object and a consensus set of properties.

At step 108 in FIG. 12, the set of properties associated with each gene locus (or gene object) is updated. At step 110 of FIG. 12, results are filtered, e.g., by performing a quality control (QC) procedure for assessing the quality of the sequence read data, the sample purity (e.g., by comparison of a sample purity to a specified sample purity threshold), successful convergence of the copy number model, and/or to assess the reliability of copy number alteration (e.g., amplifications or gains, deletions or losses) calls for individual gene loci, etc., and prepared for reporting.

In some embodiments, detecting amplifications or gains, or deletions or losses of one or more gene loci comprise using as input coverage ratio data, allele fraction data, segmentation data, and copy number model data derived by pre-processing of sequence read data for a plurality of sequence reads that overlap one or more gene loci within one or more subgenomic intervals in the sample to be analyzed. In some embodiments, amplifications or copy number gains in or more gene loci are identified on a segment-by-segment basis by comparing the copy number (CN) predicted for a gene locus (or the segment with which it is associated) by the copy number model to the ploidy of the sample as determined by the copy number model. For example, if the copy number of the gene locus (or the segment with which it is associated) is greater than the ploidy, the gene locus is determined to have been amplified or to have a copy number gain. In some embodiments, determination of amplification or copy number gain for the gene locus comprises determining if the copy number for the gene locus (or the corresponding segment) is greater than or equal to the ploidy of the sample. In some embodiments, determination of amplification or copy number gain for the gene locus comprises determining if the copy number for the gene locus (or the corresponding segment) is greater than or equal to the ploidy of the sample plus a predetermined value. In some embodiments, determination of amplification or copy number gain for the gene locus comprises determining if the copy number for the gene locus (or the corresponding segment) is greater than the ploidy of the sample. In some embodiments, determination of amplification or copy number gain for the gene locus comprises determining if the copy number for the gene locus (or the corresponding segment) is greater than the ploidy of the sample plus a predetermined value. In some embodiments, homozygous deletions of gene loci are identified on a segment-by-segment basis by determining a total copy number (total CN) for a given gene locus, and comparing the total copy number of the gene locus to a predefined value. The total copy number for the gene locus is equal to the sum of the copy numbers for a first allele and a second allele at the gene locus (e.g., a major allele and a minor allele). In some embodiments, heterozygous deletions of gene loci are identified on a segment-by-segment basis by comparing the copy numbers for a first allele and a second allele (e.g., a major allele and a minor allele) of a given gene locus to a predefined value. In some embodiments, partial deletions of gene loci may be identified by determining if the log 2 coverage ratios (“log 2 ratios” or “L2Rs”) for neighboring gene loci, single nucleotide polymorphisms (SNPs), and/or introns are significantly different from the L2R for a given gene locus, and if the L2R for the given gene locus is significantly different from a distribution of L2Rs for non-neighboring gene loci, single nucleotide polymorphisms (SNPs), and/or introns. In some embodiments, duplicate calls for gene loci (represented digitally as “gene objects”) may be merged. Upon merging, two or more gene objects and their corresponding properties (e.g., sequence location, target allele sequences, coverage ratios, etc.) will be replaced by a single gene object and a consensus set of properties. In some embodiments, the set of properties associated with each gene locus (or gene object) may be updated. In some embodiments, copy number alteration results are filtered, e.g., by performing a quality control (QC) procedure for assessing the quality of the sequence read data, the sample purity (e.g., by comparison of a sample purity to a specified sample purity threshold), successful convergence of the copy number model, and/or to assess the reliability of copy number alteration calls for individual gene loci, etc., and prepared for reporting.

In some embodiments, a CD274 gene copy number gain in a cancer, e.g., assessed in a sample from an individual (e.g., as determined according to the methods described herein), comprises a CD274 gene copy number greater than the ploidy of the sample from the individual. In some embodiments, a CD274 gene copy number gain in a cancer, e.g., assessed in a sample from an individual (e.g., as determined according to the methods described herein), comprises a CD274 gene copy number of any of at least +1, at least +2, at least +3, at least +4, at least +5, at least +6, at least +7, at least +8, at least +9, at least +10, at least +11, at least +12, at least +13, at least +14, at least +15, at least +16, at least +17, at least +18, at least +19, at least +20, at least +21, at least +22, at least +23, at least +24, at least +25, at least +26, at least +27, at least +28, at least +29, at least +30, at least +31, at least +32, at least +33, at least +34, at least +35, at least +36, at least +37, at least +38, at least +39, at least +40, at least +41, at least +42, at least +43, at least +44, at least +45, at least +46, at least +47, at least +48, at least +49, at least +50, at least +51, at least +52, at least +53, at least +54, at least +55, at least +56, at least +57, at least +58, at least +59, at least +60, at least +61, at least +62, at least +63, at least +64, at least +65, at least +66, at least +67, at least +68, at least +69, at least +70, at least +71, at least +72, at least +73, at least +74, at least +75, at least +76, at least +77, at least +78, at least +79, at least +80, at least +81, at least +82, at least +83, at least +84, at least +85, at least +86, at least +87, at least +88, at least +89, at least +90, at least +91, at least +92, at least +93, at least +94, at least +95, at least +96, at least +97, at least +98, at least +99, at least +100, or more, as compared to the ploidy of the sample from the individual. In some embodiments, a CD274 gene copy number gain in a cancer, e.g., assessed in a sample from an individual, comprises a CD274 gene copy number of at least +1, as compared to the ploidy of the sample from the individual. In some embodiments, a CD274 gene copy number gain in a cancer, e.g., assessed in a sample from an individual, comprises a CD274 gene copy number of at least +2, as compared to the ploidy of the sample from the individual. In some embodiments, the ploidy of the sample from the individual is determined using any suitable method known in the art, such as the methods described in Sun et al., PLoS Comput Biol. 2018 Feb. 7; 14(2):e1005965. In some embodiments, the ploidy of the sample from the individual is monoploid, diploid, triploid, or tetraploid. In some embodiments, the ploidy of the sample from the individual is diploid. In some embodiments, a CD274 gene copy number gain of +1 in a cancer, assessed in a sample from an individual, wherein the ploidy of the sample from the individual is diploid, is a CD274 gene copy number of at least 3 copies in the cancer. In some embodiments, a CD274 gene copy number gain of +2 in a cancer, assessed in a sample from an individual, wherein the ploidy of the sample from the individual is diploid, is a CD274 gene copy number of at least 4 copies in the cancer.

In some embodiments, a CD274 gene copy number gain in a cancer or tumor, e.g., assessed in a sample from an individual (e.g., as determined according to the methods described herein), comprises a CD274 gene copy number greater than the ploidy of the cancer or tumor. In some embodiments, a CD274 gene copy number gain in a cancer or tumor, e.g., assessed in a sample from an individual (e.g., as determined according to the methods described herein), comprises a CD274 gene copy number of any of at least +1, at least +2, at least +3, at least +4, at least +5, at least +6, at least +7, at least +8, at least +9, at least +10, at least +11, at least +12, at least +13, at least +14, at least +15, at least +16, at least +17, at least +18, at least +19, at least +20, at least +21, at least +22, at least +23, at least +24, at least +25, at least +26, at least +27, at least +28, at least +29, at least +30, at least +31, at least +32, at least +33, at least +34, at least +35, at least +36, at least +37, at least +38, at least +39, at least +40, at least +41, at least +42, at least +43, at least +44, at least +45, at least +46, at least +47, at least +48, at least +49, at least +50, at least +51, at least +52, at least +53, at least +54, at least +55, at least +56, at least +57, at least +58, at least +59, at least +60, at least +61, at least +62, at least +63, at least +64, at least +65, at least +66, at least +67, at least +68, at least +69, at least +70, at least +71, at least +72, at least +73, at least +74, at least +75, at least +76, at least +77, at least +78, at least +79, at least +80, at least +81, at least +82, at least +83, at least +84, at least +85, at least +86, at least +87, at least +88, at least +89, at least +90, at least +91, at least +92, at least +93, at least +94, at least +95, at least +96, at least +97, at least +98, at least +99, at least +100, or more, as compared to the ploidy of the cancer or tumor. In some embodiments, a CD274 gene copy number gain in a cancer, e.g., assessed in a sample from an individual, comprises a CD274 gene copy number of at least +1, as compared to the ploidy of the cancer or tumor. In some embodiments, a CD274 gene copy number gain in a cancer, e.g., assessed in a sample from an individual, comprises a CD274 gene copy number of at least +2, as compared to the ploidy of the cancer or tumor. In some embodiments, the ploidy of the cancer or tumor is determined using any suitable method known in the art, such as the methods described in Sun et al., PLoS Comput Biol. 2018 Feb. 7; 14(2):e1005965. In some embodiments, the ploidy of the cancer or tumor is monoploid, diploid, triploid, or tetraploid. In some embodiments, the ploidy of the cancer or tumor is diploid. In some embodiments, a CD274 gene copy number gain of +1 in a cancer, assessed in a sample from an individual, wherein the ploidy of the cancer or tumor is diploid, is a CD274 gene copy number of at least 3 copies in the cancer. In some embodiments, a CD274 gene copy number gain of +2 in a cancer, assessed in a sample from an individual, wherein the ploidy of the cancer or tumor is diploid, is a CD274 gene copy number of at least 4 copies in the cancer.

In some embodiments, a CD274 gene copy number gain in a cancer, e.g., assessed in a sample from an individual (e.g., as determined according to the methods described herein), comprises a CD274 gene copy number of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is monoploid. In some embodiments, a CD274 gene copy number gain in a cancer, e.g., assessed in a sample from an individual comprises a CD274 gene copy number of at least 2, wherein the ploidy of the sample from the individual is monoploid.

In some embodiments, a CD274 gene copy number gain in a cancer, e.g., assessed in a sample from an individual (e.g., as determined according to the methods described herein), comprises a CD274 gene copy number of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is diploid. In some embodiments, a CD274 gene copy number gain in a cancer, e.g., assessed in a sample from an individual comprises a CD274 gene copy number of at least 3, wherein the ploidy of the sample from the individual is diploid. In some embodiments, a CD274 gene copy number gain in a cancer, e.g., assessed in a sample from an individual comprises a CD274 gene copy number of at least 4, wherein the ploidy of the sample from the individual is diploid.

In some embodiments, a CD274 gene copy number gain in a cancer, e.g., assessed in a sample from an individual (e.g., as determined according to the methods described herein), comprises a CD274 gene copy number of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is triploid. In some embodiments, a CD274 gene copy number gain in a cancer, e.g., assessed in a sample from an individual comprises a CD274 gene copy number of at least 4, wherein the ploidy of the sample from the individual is triploid. In some embodiments, a CD274 gene copy number gain in a cancer, e.g., assessed in a sample from an individual comprises a CD274 gene copy number of at least 6, wherein the ploidy of the sample from the individual is triploid.

In some embodiments, a CD274 gene copy number gain in a cancer, e.g., assessed in a sample from an individual (e.g., as determined according to the methods described herein), comprises a CD274 gene copy number of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is tetraploid. In some embodiments, a CD274 gene copy number gain in a cancer, e.g., assessed in a sample from an individual comprises a CD274 gene copy number of at least 5, wherein the ploidy of the sample from the individual is tetraploid. In some embodiments, a CD274 gene copy number gain in a cancer, e.g., assessed in a sample from an individual comprises a CD274 gene copy number of at least 8, wherein the ploidy of the sample from the individual is tetraploid.

In some embodiments, a CD274 gene copy number gain in a cancer, e.g., assessed in a sample from an individual (e.g., as determined according to the methods described herein), comprises a CD274 gene copy number of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor from the individual is monoploid. In some embodiments, a CD274 gene copy number gain in a cancer, e.g., assessed in a sample from an individual comprises a CD274 gene copy number of at least 2, wherein the ploidy of the cancer or tumor from the individual is monoploid.

In some embodiments, a CD274 gene copy number gain in a cancer, e.g., assessed in a sample from an individual (e.g., as determined according to the methods described herein), comprises a CD274 gene copy number of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is diploid. In some embodiments, a CD274 gene copy number gain in a cancer, e.g., assessed in a sample from an individual comprises a CD274 gene copy number of at least 3, wherein the ploidy of the cancer or tumor is diploid. In some embodiments, a CD274 gene copy number gain in a cancer, e.g., assessed in a sample from an individual comprises a CD274 gene copy number of at least 4, wherein the ploidy of the cancer or tumor is diploid.

In some embodiments, a CD274 gene copy number gain in a cancer, e.g., assessed in a sample from an individual (e.g., as determined according to the methods described herein), comprises a CD274 gene copy number of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is triploid. In some embodiments, a CD274 gene copy number gain in a cancer, e.g., assessed in a sample from an individual comprises a CD274 gene copy number of at least 4, wherein the ploidy of the cancer or tumor is triploid. In some embodiments, a CD274 gene copy number gain in a cancer, e.g., assessed in a sample from an individual comprises a CD274 gene copy number of at least 6, wherein the ploidy of the cancer or tumor is triploid.

In some embodiments, a CD274 gene copy number gain in a cancer, e.g., assessed in a sample from an individual (e.g., as determined according to the methods described herein), comprises a CD274 gene copy number of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is tetraploid. In some embodiments, a CD274 gene copy number gain in a cancer, e.g., assessed in a sample from an individual comprises a CD274 gene copy number of at least 5, wherein the ploidy of the cancer or tumor is tetraploid. In some embodiments, a CD274 gene copy number gain in a cancer, e.g., assessed in a sample from an individual comprises a CD274 gene copy number of at least 8, wherein the ploidy of the cancer or tumor is tetraploid.

In some embodiments of any of the methods provided herein, the methods may comprise one or more of the steps of: (i) obtaining a sample from an individual (e.g., an individual having, suspected of having, or determined to have cancer), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adapter sequences, substrate adapter sequences, sample index sequences, or unique molecular identifier (UMI) sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, wherein the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual having, suspected of having or determined to have cancer), wherein the plurality of nucleic acid molecules comprises nucleic acid molecules corresponding to a CD274 gene; (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to a CD274 gene; (f) analyzing the plurality of sequence reads to determine presence or absence of a CD274 gene copy number alteration, such as CD274 gene copy number gain, e.g., as described above; and (g) based on the analysis, detecting the presence or absence of a CD274 gene copy number alteration, such as CD274 gene copy number gain, in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to a CD274 gene. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual having, suspected of having or determined to have cancer), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a CD274 gene in said library to produce an enriched sample; (e) sequencing the enriched sample, thereby producing a plurality of sequence reads; (f) analyzing the plurality of sequence reads to determine presence or absence of a CD274 gene copy number alteration, such as CD274 gene copy number gain, e.g., as described above; (g) detecting, based on the analyzing step, the presence or absence of the CD274 gene copy number alteration, such as CD274 gene copy number gain, in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample; and the non-cancer nucleic acid molecules are derived from a non-tumor fraction of the liquid biopsy sample or a cell-free DNA (cfDNA) fraction of the liquid biopsy sample.

In some embodiments of any of the methods, the one or more adapters comprise amplification primers, flow cell adaptor sequences, substrate adapter sequences, sample index sequences, or unique molecular identifier (UMI) sequences. In some embodiments, the one or more adapters comprise one or more sample index sequences. As is known in the art, sample indexes allow the sequencing of multiple samples on the same instrument flow cell or chip (i.e., multiplexing). Sample indexes are typically between about 8 and about 10 bases in length, and comprise a nucleotide sequence specific to a sample that is used to assign sequence reads to the correct sample during data analysis. In some embodiments, the one or more adapters comprise one or more unique molecule identifiers (UMIs). As is known in the art, UMIs comprise short nucleotide sequences that include a unique barcode that is incorporated into each molecule in a given sample library. UMIs are useful for identifying PCR duplicates created during library amplification steps, and/or for reducing the rate of false-positive variant calls and increasing variant detection, since variant alleles present in the original sample (true variants) can be distinguished from errors introduced during library preparation, target enrichment, or sequencing.

In some embodiments, the selectively enriching comprises: (a) combining one or more bait molecules with the library, thereby hybridizing the one or more bait molecules to one or more nucleic acid molecules and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer.

In some embodiments of any of the methods provided herein, the methods further comprise selectively enriching for one or more nucleic acids in the sample comprising nucleotide sequences corresponding to CD274. In some embodiments, the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to CD274 and producing nucleic acid hybrids; and (b) isolating the nucleic acid hybrids to produce the enriched sample. In some embodiments, the selectively enriching comprises amplifying the one or more nucleic acids comprising nucleotide sequences corresponding to CD274 using a polymerase chain reaction (PCR) to produce an enriched sample. In some embodiments, the methods further comprise sequencing the enriched sample.

In some embodiments of any of the methods for detection of CD274 gene copy number alterations, such as CD274 gene copy number gains, provided herein (see, e.g., sections i-iv herein), the methods further comprise analyzing sequence data (e.g., obtained from sequencing as described above), for the presence or absence of one or more alterations (e.g., a base substitution, a short insertion/deletion (indel), a copy number alteration, or a genomic rearrangement) in one or more genes (e.g., one or more cancer-related genes such as EGFR, ALK and/or CD274, or a panel of known/suspected oncogenes and/or tumor suppressors, or any combination thereof). In some embodiments, the presence or absence of one or more gene alterations of the disclosure is detected using any suitable method known in the art, e.g., as described in Frampton et al., (2013) Nat Biotechnol, 31:1023-1031. In some embodiments, base substitution alterations are detected using Bayesian methodology, which allows detection of novel somatic mutations at low mutant allele frequency (MAF) and increased sensitivity for mutations at hotspot sites through the incorporation of tissue-specific prior expectations. See, e.g., Kim et al., Cancer Discov (2011) 1:44-53 and Frampton et al., (2013) Nat Biotechnol, 31:1023-1031. In some embodiments, insertion/deletion (indel) alterations are detected using any suitable method, such as de novo local assembly, e.g., using the de Bruijn approach, see, e.g., Compeau et al., Nat Biotechnol (2011) 29:987-991 and Frampton et al., (2013) Nat Biotechnol, 31:1023-1031. In some embodiments, gene fusion and genomic rearrangement alterations are detected using any suitable method, such as by analyzing chimeric read pairs (read pairs for which reads map to separate chromosomes, or at a distance of over 10 Mbp), see, e.g., Frampton et al., (2013) Nat Biotechnol, 31:1023-1031. In some embodiments, rearrangements are annotated for predicted function (e.g., creation of fusion gene or tumor suppressor inactivation).

In some embodiments of any of the methods for detection of CD274 gene copy number alterations, such as CD274 gene copy number gains, provided herein (see, e.g., sections i-iv herein), the methods further comprise generating a molecular profile for the individual or the sample, based, at least in part, on detecting the presence or absence of the CD274 gene copy number alteration, such as CD274 gene copy number gain. In some embodiments, the molecular profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test. In some instances, a molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some embodiments of any of the methods for detection of CD274 gene copy number alterations, such as CD274 gene copy number gains, provided herein (see, e.g., sections i-iv herein), the methods further comprise selecting a treatment, administering a treatment, or applying a treatment to the individual based on the generated molecular profile, wherein the treatment comprises an anti-cancer therapy, e.g., as described herein, e.g., an immunotherapy. In some embodiments of any of the methods for detection of CD274 gene copy number alterations, such as CD274 gene copy number gains, provided herein (see, e.g., sections i-iv herein), the methods further comprise generating a report indicating the presence or absence of a CD274 gene copy number alteration, such as CD274 gene copy number gain, in the sample. In some embodiments of any of the methods for detection of CD274 gene copy number alterations, such as CD274 gene copy number gains, provided herein (see, e.g., sections i-iv herein), the methods further comprise generating, by one or more processors, a report indicating the presence or absence of a CD274 gene copy number alteration, such as CD274 gene copy number gain, in the sample. In some embodiments, the report comprises the generated molecular profile. In some embodiments, the methods further comprise providing or transmitting the report, e.g., as described below. In some embodiments, the report is transmitted via a computer network or a peer-to-peer connection. In some instances, all or a portion of the report may be displayed in a graphical user interface of an online or web-based healthcare portal.

In some embodiments of any of the methods for detection of CD274 gene copy number alterations, such as CD274 gene copy number gains, provided herein (see, e.g., sections i-iv herein), the methods for determining the presence or absence of a CD274 gene copy number alteration, such as CD274 gene copy number gain, may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of a CD274 gene copy number alteration, such as CD274 gene copy number gain, as part of a genomic profiling process can improve the validity of, e.g., disease detection calls by, for example, independently confirming the presence of the CD274 gene copy number alteration, such as CD274 gene copy number gain, in a given patient sample.

The disclosed methods may be used with any of a variety of samples, e.g., as described in further detail below. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof. In some instances, the nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample. In some embodiments of any of the methods provided herein, the method further comprises determining the circulating tumor DNA (ctDNA) fraction of a liquid biopsy sample.

In some embodiments, one or more of the copies of the CD274 gene, e.g., in a cancer or tumor, comprise the entire CD274 gene. In some embodiments, one or more of the copies of the CD274 gene comprise a portion of the CD274 gene. In some embodiments, one or more of the copies of the CD274 gene encode a PD-L1 polypeptide or a fragment thereof. In some embodiments, one or more of the copies of the CD274 gene encode a PD-L1 polypeptide or a functional fragment thereof.

### (v) Probes, Baits and Oligonucleotides

Also provided herein are probes, baits and oligonucleotides suitable for the detection of a CD274 gene copy number alteration, such as CD274 gene copy number gain, e.g., according to any methods of detection known in the art and/or described herein.

### Probes

Provided herein are probes suitable for the detection of a CD274 gene copy number alteration, such as CD274 gene copy number gain, e.g., according to any methods of detection known in the art and/or described herein.

In some embodiments, a probe provided herein comprises a nucleic acid sequence configured to hybridize to a target nucleic acid molecule (e.g., corresponding to one or more genes, such as CD274), or a portion thereof. In some embodiments, the probe comprises a nucleotide sequence configured to hybridize to a nucleotide sequence in an intron or an exon of a target gene, such as a CD274 gene. In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization. In some embodiments, probes provided herein may be used according to the methods of detection of a CD274 gene copy number alteration, such as CD274 gene copy number gain, provided herein. For example, a probe provided herein may be used for detecting a CD274 gene copy number alteration, such as CD274 gene copy number gain, in a sample, e.g., a sample obtained from an individual. In some embodiments, the probe may be used for identifying cells or tissues that comprise a CD274 gene copy number alteration, such as CD274 gene copy number gain. In some embodiments, one or more probes provided herein are suitable for use in in situ hybridization methods, e.g., as described above, such as FISH.

Chromosomal probes, e.g., for use in the FISH methods described herein, are typically about 50 to about 105 nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as a CD274 gene product (e.g., a PD-L1 mRNA).

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that is or results in a CD274 gene copy number alteration, such as CD274 gene copy number gain. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, insertions, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules may be used, or further processing may be performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

### Baits

Provided herein are baits suitable for the detection of a CD274 gene copy number alteration, such as CD274 gene copy number gain, e.g., according to any methods of detection known in the art and/or described herein.

In some embodiments of the methods provided herein, nucleic acid molecules (e.g., corresponding to one or more genes, such as CD274) are captured (e.g., from amplified nucleic acids) by hybridization with a bait molecule. In some embodiments, a bait molecule comprises a capture nucleic acid molecule configured to hybridize to a target nucleic acid molecule, or a fragment or portion thereof. In some embodiments, the capture nucleic acid molecule is configured to hybridize to a fragment of a target (e.g., a fragment of one or more genes, such as CD274). In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule is configured to hybridize to a nucleotide sequence in an intron or an exon of a gene, e.g., a CD274 gene. In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein includes a label, a tag or detection reagent. In some embodiments, the label, tag or detection reagent is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides in length. In some embodiments, a bait provided herein is between about 130 nucleotides and about 230 nucleotides in length. In some embodiments, a bait provided herein is between about 150 nucleotides and about 200 nucleotides in length. In some embodiments, a bait provided herein comprises a target-specific bait sequence and universal tails on each end. In some embodiments, the target-specific sequence is between about 40 nucleotides and about 300 nucleotides in length. In some embodiments, the target-specific sequence is between about 100 nucleotides and about 200 nucleotides in length. In some embodiments, the target-specific sequence is between about 120 nucleotides and about 170 nucleotides in length. In some embodiments, the target-specific sequence is about 150 nucleotides or about 170 nucleotides in length. In some embodiments, a bait provided herein comprises an oligonucleotide comprising about 200 nucleotides, of which about 150 nucleotides or about 170 nucleotides are target-specific, and the other 50 nucleotides or 30 nucleotides (e.g., 25 or 15 nucleotides on each end of the bait) are universal arbitrary tails, e.g., suitable for PCR amplification.

The baits described herein can be used for selection of exons and short target sequences. In some embodiments, a bait of the disclosure distinguishes a target nucleic acid molecule, e.g., a genomic or transcribed nucleic acid molecule, e.g., a cDNA or RNA from a reference nucleotide sequence.

### Oligonucleotides

Provided herein are oligonucleotides, e.g., useful as primers, suitable for the detection of a CD274 gene copy number alteration, such as CD274 gene copy number gain, e.g., according to any methods of detection known in the art and/or described herein.

In some embodiments, an oligonucleotide, e.g., a primer, provided herein comprises a nucleotide sequence configured to hybridize to a target nucleic acid molecule (e.g., corresponding to a gene, such as a CD274 gene), or a fragment or portion thereof. In some embodiments, the oligonucleotide comprises a nucleotide sequence configured to hybridize to a CD274 gene or a fragment thereof. In some embodiments, the oligonucleotide, e.g., the primer, comprises a nucleotide sequence configured to hybridize to a nucleotide sequence in an intron or an exon of a gene (e.g., a CD274 gene), or a fragment thereof. In some embodiments, the oligonucleotide comprises a nucleotide sequence corresponding to a gene, such as a CD274 gene. In some embodiments, the oligonucleotide comprises a nucleotide sequence corresponding to a fragment or a portion of a gene, such as a CD274 gene. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to the sequence of a gene, such as a CD274 gene. In some embodiments, the oligonucleotide comprises a nucleotide sequence complementary to a fragment or a portion of the sequence of a gene, such as a CD274 gene. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a target nucleic acid molecule (e.g., a gene such as a CD274 gene, or a portion thereof), e.g., using PCR. In some embodiments, the oligonucleotide may be used to sequence a target nucleic acid molecule (e.g., a gene such as a CD274 gene, or a portion thereof). In some embodiments, pairs of oligonucleotides, e.g., pairs of primers, are provided herein, which are configured to hybridize to a target nucleic acid molecule (e.g., a gene such as a CD274 gene, or a portion thereof), or a fragment thereof. In some embodiments, a pair of oligonucleotides of the disclosure may be used for directing amplification of a target nucleic acid molecule (e.g., a gene such as a CD274 gene, or a portion thereof), or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In one aspect, provided herein is a primer or primer set for amplifying a nucleic acid molecule comprising a cytogenetic abnormality that is or results in a CD274 gene copy number alteration, such as CD274 gene copy number gain. The cytogenetic abnormality may be any cytogenetic abnormality that is or results in a CD274 gene copy number alteration, such as CD274 gene copy number gain. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, insertions, gene copy number changes, germ-line mutations, and gene expression level changes.

### B. Tumor Mutational Burden

In some embodiments, the methods provided herein comprise acquiring knowledge of or detecting the level of tumor mutational burden in a cancer of the disclosure.

As demonstrated herein, CD274 gene copy number alterations, such as CD274 gene copy number gains, in cancer with high tumor mutational burden may be predictive of increased survival and/or increased likelihood of response when treated with an immunotherapy, such as an immune checkpoint inhibitor. See, e.g., Example 1. Accordingly, in some embodiments, provided herein are methods that comprise acquiring knowledge of or detecting high tumor mutational burden in a cancer. In some embodiments, the methods of the disclosure also comprise acquiring knowledge of or detecting CD274 gene copy number alterations, such as CD274 gene copy number gains, in the cancer (see, e.g., Section A, above).

In some embodiments, acquiring knowledge of or detecting the level of tumor mutational burden in a cancer of the disclosure comprises measuring the level of tumor mutational burden in a sample, e.g., in a sample from a cancer or a tumor, obtained from an individual.

In some embodiments, tumor mutational burden is assessed in sample from an individual, such as sample described herein. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, DNA, circulating tumor DNA, cell-free DNA, or cell-free RNA.

In some embodiments, tumor mutational burden is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety. In some embodiments, tumor mutational burden is measured according to the methods described in Montesion, M., et al., Cancer Discovery (2021) 11(2):282-92.

In some embodiments, tumor mutational burden is measured according to the methods described in Chalmers et al., Genome Med (2017) 19; 9(1):34. In some embodiments, tumor mutational burden is assessed as the number of somatic, coding, base substitution, and indel mutations per megabase of genome examined. In some embodiments, all base substitutions and indels in the coding regions of targeted genes, including synonymous alterations, are counted. In some embodiments, assessment of tumor mutational burden further comprises filtering/counting of the base substitutions and indels, comprising one or more, or all, of the following steps: (a) synonymous mutations are counted in order to reduce sampling noise; (b) non-coding alterations are not counted; (c) alterations listed as known somatic alterations in COSMIC (see, e.g., cancer.sanger.ac.uk/cosmic) and truncations in tumor suppressor genes are not counted; (d) alterations predicted to be germline by a somatic-germline zygosity algorithm (Sun et al., Cancer Res. 2014; 74(19S):1893) are not counted; (e) alterations that are recurrently predicted to be germline are not counted; (f) known germline alterations in dbSNP (see, e.g., www.ncbi.nlm.nih.gov/snp/) are not counted; and (g) germline alterations occurring with two or more counts in the ExAC database (Lek et al., Nature. 2016; 536:285-91.) are not counted. In some embodiments, to calculate the tumor mutational burden per megabase, the total number of mutations counted (e.g., as described above) is divided by the size of the coding region of the targeted territory. In some embodiments, the nonparametric Mann-Whitney U test is used to test for significance in difference of means between two populations.

In some embodiments, tumor mutational burden is assessed based on the number of non-driver somatic coding mutations/megabase (mut/Mb) of genome sequenced.

In some embodiments, tumor mutational burden is measured in the sample by whole exome sequencing. In some embodiments, tumor mutational burden is measured in the sample using next-generation sequencing. In some embodiments, tumor mutational burden is measured in the sample using whole genome sequencing. In some embodiments, tumor mutational burden is measured in the sample by gene-targeted sequencing. In some embodiments, tumor mutational burden is measured on between about 0.7 Mb and about 1.3 Mb of sequenced DNA. In some embodiments, tumor mutational burden is measured on any of about 0.7 Mb, about 0.75 Mb, about 0.79 Mb, about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, about 1.1 Mb, about 1.2 Mb, or about 1.3 Mb of sequenced DNA. In some embodiments, tumor mutational burden is measured on about 0.79 Mb of sequenced DNA. In some embodiments, tumor mutational burden is measured on between about 0.83 Mb and about 1.14 Mb of sequenced DNA. In some embodiments, tumor mutational burden is measured on about 0.8 Mb of sequenced DNA. In some embodiments, tumor mutational burden is measured on between about 0.83 Mb and about 1.14 Mb of sequenced DNA. In some embodiments, tumor mutational burden is measured on up to about 1.24 Mb of sequenced DNA. In some embodiments, tumor mutational burden is measured on up to about 1.1 Mb of sequenced DNA.

In some embodiments, a cancer of the disclosure has a high tumor mutational burden, wherein the cancer has a tumor mutational burden of at least about 5 mut/Mb. In some embodiments, a cancer of the disclosure has a high tumor mutational burden, wherein the cancer has a tumor mutational burden of at least about 10 mut/Mb. In some embodiments, the cancer has a tumor mutational burden of at least about 20 mut/Mb. In some embodiments, the cancer has a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the cancer has a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, the cancer has a TMB of at least about 100 mut/Mb, at least about 110 mut/Mb, at least about 120 mut/Mb, at least about 130 mut/Mb, at least about 140 mut/Mb, at least about 150 mut/Mb, or more.

In some embodiments, measuring tumor mutational burden comprises assessing mutations in a sample derived from a cancer in an individual. In some embodiments, measuring tumor mutational burden comprises assessing mutations in a sample derived from a cancer in an individual and in a matched normal sample, e.g., a sample from the individual derived from a tissue or other source that is free of the cancer.

In some embodiments, tumor mutational burden is obtained from a plurality of sequence reads, e.g., a plurality of sequence reads obtained by sequencing nucleic acids corresponding to at least a portion of a genome (such as from an enriched or unenriched sample), e.g., according to any sequencing method known in the art or described herein (e.g., as described above, in Section A). In some embodiments, tumor mutational burden is determined based on the number of non-driver somatic coding mutations per megabase of genome sequenced.

In some embodiments, any of the methods of the present disclosure comprise acquiring knowledge of CD274 gene copy number alterations, such as CD274 gene copy number gains (e.g., in a sample obtained from an individual), and acquiring knowledge of tumor mutational burden (e.g., in a sample obtained from an individual). In some embodiments, any of the methods of the present disclosure comprise detecting CD274 gene copy number alterations, such as CD274 gene copy number gains (e.g., in a sample obtained from an individual), and acquiring knowledge of tumor mutational burden (e.g., in a sample obtained from an individual). In some embodiments, any of the methods of the present disclosure comprise acquiring knowledge of CD274 gene copy number alterations, such as CD274 gene copy number gains (e.g., in a sample obtained from an individual), and detecting or determining tumor mutational burden (e.g., in a sample obtained from an individual). In some embodiments, any of the methods of the present disclosure comprise detecting CD274 gene copy number alterations, such as CD274 gene copy number gains (e.g., in a sample obtained from an individual), and detecting or determining tumor mutational burden (e.g., in a sample obtained from an individual).

In some embodiments of any of the methods of the disclosure, the samples used to detect/determine CD274 gene copy number alterations, such as CD274 gene copy number gains, and tumor mutational burden are the same (i.e., CD274 gene copy number alterations, such as CD274 gene copy number gains, and tumor mutational burden are detected/determined in one sample). In some embodiments of any of the methods of the disclosure, the samples used to detect/determine CD274 gene copy number alterations, such as CD274 gene copy number gains, and tumor mutational burden are different (i.e., CD274 gene copy number alterations, such as CD274 gene copy number gains, are detected/determined in one sample; and tumor mutational burden is detected/determined in another sample).

### C. PD-L1 Expression

In some embodiments, the methods provided herein comprise acquiring knowledge of or detecting the level of PD-L1 expression in a cancer of the disclosure. In some embodiments, acquiring knowledge of or detecting the level of PD-L1 expression in a cancer of the disclosure comprises measuring PD-L1 expression in a sample, e.g., in a sample from a cancer obtained from an individual.

Any suitable method for measuring PD-L1 expression in a sample from an individual may be used. For example, the level of PD-L1 expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used.

In some embodiments, PD-L1 expression in a sample from an individual is measured based on the level of PD-L1 mRNA in the sample. Any suitable method for measuring mRNA expression in a sample from an individual may be used. For example, the level of PD-L1 mRNA expression may be measured using in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”).

In some embodiments, PD-L1 expression in a sample from an individual is measured based on the level of PD-L1 protein in the sample. Any suitable method for measuring protein expression in a sample from an individual may be used. For example, the level of PD-L1 protein expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), proteomics (e.g., mass spectrometry), quantitative blood based assays (as for example serum ELISA), biochemical enzymatic activity assays, or multiplexed immunoassays such as those available from Rules Based Medicine or Meso Scale Discovery (“MSD”).

In some embodiments, PD-L1 expression is measured by immunohistochemistry using commercially available antibody clones 22C3 (Dako/Agilent) or SP142 (Ventana), e.g., according to methods known in the art and/or described herein.

In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs), e.g., in a sample from an individual, express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in the sample express PD-L1 protein and/or PD-L1 mRNA (e.g., are positive for PD-L1 protein and/or PD-L1 mRNA). In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of the tumor area is occupied by PD-L1-expressing tumor-infiltrating immune cells. In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of the tumor area is occupied by PD-L1-expressing tumor-infiltrating immune cells.

In some embodiments, the level of PD-L1 protein and/or PD-L1 mRNA is assessed in a sample from an individual, such as a sample described herein. In some embodiments, the sample from the individual comprises fluid, cells, or tissue. In some embodiments, the sample from the individual comprises a tumor biopsy or a circulating tumor cell. In some embodiments, the sample is obtained or derived from a cancer or the disclosure.

In some embodiments of any of the methods provided herein, a sample from an individual, e.g., an individual having a cancer, is determined to be PD-L1-negative if less than 1% of tumor cells in the sample express PD-L1. In some embodiments of any of the methods provided herein, a sample from an individual having a cancer is determined to be PD-L1 positive if at least about 1% of tumor cells in the sample express PD-L1.

In some embodiments, the level of PD-L1 protein expression is measured using a VENTANA PD-L1 assay (SP142). In some embodiments, the level of PD-L1 protein expression is determined based on PD-L1 expression in tumor infiltrating immune cells (ICs) and/or tumor cells (TCs) using a VENTANA PD-L1 assay (SP142). Additional information about the VENTANA SP142 assay may be found in the website: www[dot]accessdata[dot]fda[dot]gov/cdrh_docs/pdfi6/PI60002c.pdf. In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs), e.g., in a sample from an individual, express PD-L1 protein (e.g., are positive for PD-L1 protein). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in the sample express PD-L1 protein (e.g., are positive for PD-L1 protein). In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of the tumor area is occupied by PD-L1-expressing tumor-infiltrating immune cells of any intensity (IC). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of the tumor area is occupied by PD-L1-expressing tumor-infiltrating immune cells of any intensity (IC).

In some embodiments, the level of PD-L1 protein expression is assessed based on a tumor proportion score (TPS). The TPS is the percentage of tumor cells showing partial or complete PD-L1 membrane staining (e.g., at a ≥1+ intensity on a 0, 1+, 2+, and 3 scale) relative to all tumor cells present in the sample. In some embodiments, the TPS is calculated as: the number of PD-L1-positive tumor cells/Total number of PD-L1-positive tumor cells +Total number of PD-L1-negative tumor cells. A PD-L1 low positive status refers to a TPS of between 1% and 49%, PD-L1 high positive status refers to a TPS of 50% or greater, and a PD-L1 negative status refers to a TPS of less than 1%. In some embodiments, a cancer of the disclosure is determined to be PD-L1 positive if it has PD-L1 low positive status or a PD-L1 high positive status. In some embodiments, a cancer of the disclosure is PD-L1 positive (e.g., the cancer is determined have a TPS of any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%, in a sample obtained from an individual having the cancer). In some embodiments, a cancer of the disclosure is PD-L1 low positive (e.g., the cancer is determined have a TPS of any of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 49%, in a sample obtained from an individual having the cancer). In some embodiments, a cancer of the disclosure is PD-L1 high positive (e.g., the cancer is determined have a TPS of any of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, in a sample obtained from an individual having the cancer). In some embodiments, a cancer of the disclosure is PD-L1 negative (e.g., the cancer is determined have a TPS of less than 1%, in a sample obtained from an individual having the cancer). In some embodiments, the TPS is determined using a DAKO 22C3 assay. Additional information about the DAKO 22C3 assay and the TPS score may be found, e.g., in the website: www[dot]agilent[dot]com/cs/library/usermanuals/public/29158_pd-11-ihc-22C3-pharmdx-nsclc-interpretation-manual.pdf.

In some embodiments, PD-L1 expression is assessed based on a combined positive score (CPS). The CPS refers to the number of PD-L1 staining cells (e.g., tumor cells, lymphocytes, or macrophages) divided by the total number of viable tumor cells, and multiplied by 100. See, e.g., www[dot]agilent[dot]com/en/product/pharmdx/pd-l1-ihc-22c3-pharmdx-overview #pink3. In some embodiments, a cancer of the disclosure has a CPS of at least about 1, at least about 2, at least about 3, at least about 4, at least about 5, at least about 6, at least about 7, at least about 8, at least about 9, or at least about 10. In some embodiments, a cancer of the disclosure has high PD-L1 expression, e.g., with a CPS of at least about 1, such as between about 1 and about 5, between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, or about 100. In some embodiments, PD-L1 expression based on CPS is assessed using a DAKO 22C3 assay. Additional information about the DAKO 22C3 assay and the CPS may be found, e.g., in the websites: www[dot]agilent[dot]com/en/product/pharmdx/pd-l1-ihc-22c3-pharmdx-overview #pink3; www[dot]agilent[dot]com/cs/library/usermanuals/public/29171_22C3-ihc-pharmdx-interpretation-manual-eu.pdf; www[dot]agilent[dot]com/cs/library/usermanuals/public/13350a_eu_urothelial_carcinoma_interpretat ion_manual_r3v9_fin_150_single.pdf.pdf; and www[dot]agilent[dot]com/cs/library/usermanuals/public/29314_22c3_pharmDx_hnsec_interpretation manual_us.pdf.

In some embodiments of any of the methods provided herein, PD-L1 expression is assessed using a companion diagnostic device, e.g., as provided in www[dot]fda[dot]gov/medical-devices/in-vitro-diagnostics/list-cleared-or-approved-companion-diagnostic-devices-in-vitro-and-imaging-tools.

### D. Microsatellite Instability Status

In some embodiments, the methods provided herein comprise acquiring knowledge of or determining a microsatellite instability status in a cancer of the disclosure. In some embodiments, acquiring knowledge of or determining microsatellite instability status in a cancer of the disclosure comprises assessing microsatellite instability status in a sample, e.g., in a sample from a cancer obtained from an individual.

Microsatellite instability may be assessed using any suitable method known in the art. For example, microsatellite instability may be measured using sequencing (e.g., a massively parallel sequencing (MPS) technique, whole genome sequencing (WGS), whole exome sequencing, targeted sequencing, direct sequencing, or a Sanger sequencing technique), next generation sequencing (see, e.g., Hempelmann et al., J Immunother Cancer (2018) 6(1):29), a PCR-based amplification technique, a non-PCR amplification technique, an isothermal amplification technique, Fluorescent multiplex PCR, capillary electrophoresis (see, e.g., Arulananda et al., J Thorac Oncol (2018) 13(10):1588-94), immunohistochemistry (see, e.g., Cheah et al., Malays J Pathol (2019) 41(2):91-100), or single-molecule molecular inversion probes (smMIPs, see, e.g., Waalkes et al., Clin Chem (2018) 64(6):950-8). In some embodiments, microsatellite instability is assessed based on DNA sequencing (e.g., next generation sequencing) of up to about 114 loci. In some embodiments, microsatellite instability is assessed based on DNA sequencing (e.g., next generation sequencing) of intronic homopolymer repeat loci for length variability. In some embodiments, microsatellite instability is assessed based on DNA sequencing (e.g., next generation sequencing) of about 114 intronic homopolymer repeat loci for length variability. In some embodiments, microsatellite instability status is determined as described in Trabucco et al., J Mol Diagn. 2019 November; 21(6):1053-1066.

In some embodiments, a cancer of the disclosure is, or is determined to be (e.g., according to any method known in the art and/or described herein), microsatellite stable. In some embodiments, a cancer of the disclosure has, or is determined to have (e.g., according to any method known in the art and/or described herein), high microsatellite instability (MSI-H).

In some embodiments, acquiring knowledge of or determining a microsatellite instability status in a cancer of the disclosure comprises determining the microsatellite instability status in a sample, e.g., in a sample from a cancer or a tumor, obtained from an individual.

In some embodiments, microsatellite instability is assessed in sample from an individual, such as a sample described herein. In some embodiments, the sample from the individual comprises fluid, cells, or tissue, e.g., from a liquid biopsy or a tissue biopsy such as a tumor biopsy, e.g., a described in greater detail herein. In some embodiments, the sample from the individual comprises a tumor biopsy or a circulating tumor cell. In some embodiments, the sample from the individual comprises nucleic acids. In some embodiments, the sample from the individual comprises mRNA, DNA, circulating tumor DNA, cell-free DNA, or cell-free RNA.

### E. Samples

A variety of materials can be the source of, or serve as, samples for use in any of the methods of the disclosure, such as any of the methods for detection of CD274 copy number alterations (e.g., CD274 copy number gains), tumor mutational burden, microsatellite instability status, or PD-L1 expression.

For example, the sample can be, or be derived from: solid tissue such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics or the like. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid, sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, nucleic acids (e.g., for use in any of the methods for detection of CD274 copy number alterations, tumor mutational burden, microsatellite instability status, or PD-L1 expression) or proteins (e.g., for use in any of the methods for detection of CD274 copy number alterations, microsatellite instability status, or PD-L1 expression provided herein) extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a cancer, such as a cancer described herein. In some embodiments, the methods provided herein comprise obtaining one or more samples from the individual (e.g., the individual having a cancer). In some embodiments, the one or more samples are obtained or derived from a cancer (e.g., a cancer in an individual). In some embodiments, the one or more samples comprise at least 20% tumor cell nuclear area.

In some embodiments, the sample is a control sample or a reference sample, e.g., not containing a CD274 gene copy number alteration and/or high tumor mutational burden. In certain embodiments, the reference sample is purified or isolated (e.g., it is removed from its natural state). In certain embodiments, the control or reference sample is from a non-tumor or cancer sample, e.g., a blood control, a normal adjacent tumor (NAT), or any other non-cancerous sample from the same or a different individual.

In some embodiments, a CD274 gene copy number alteration (e.g., a CD274 gene copy number gain) and/or high tumor mutational burden are detected in a sample comprising genomic or subgenomic DNA fragments, or RNA (e.g., mRNA), isolated from a sample, e.g., a tumor or cancer sample, a normal adjacent tissue (NAT) sample, a tissue sample, or a blood, plasma, cerebrospinal fluid, sputum, stool, urine, or saliva sample obtained from an individual. In some embodiments, the sample comprises cDNA derived from an mRNA sample or from a sample comprising mRNA. In some embodiments, a CD274 gene copy number alteration (e.g., a CD274 gene copy number gain) and/or high tumor mutational burden are detected in a sample comprising cell-free DNA (cfDNA), cell-free RNA, and/or circulating tumor DNA (ctDNA). In some embodiments, a CD274 gene copy number alteration (e.g., a CD274 gene copy number gain) and/or high tumor mutational burden are detected in a sample comprising cell-free DNA (cfDNA) and/or circulating tumor DNA (ctDNA). In some embodiments, a CD274 gene copy number alteration (e.g., a CD274 gene copy number gain) and/or high tumor mutational burden are detected in a sample comprising circulating tumor DNA (ctDNA).

In some embodiments, any of the methods of the present disclosure comprise acquiring knowledge of or detecting any of the biomarkers described herein (e.g., CD274 gene copy number alterations, such as CD274 gene copy number gains, tumor mutational burden, PD-L1 expression, and/or microsatellite instability status) in one or more samples (e.g., as described above) obtained from an individual (e.g., an individual having a cancer). In some embodiments, the samples used to acquire knowledge of or detect any of the biomarkers described herein (e.g., CD274 gene copy number alterations, such as CD274 gene copy number gains, tumor mutational burden, PD-L1 expression, and/or microsatellite instability status) are the same sample (i.e., one or more, or all, of CD274 gene copy number alterations, such as CD274 gene copy number gains, tumor mutational burden, PD-L1 expression, and/or microsatellite instability status are detected or determined in one sample). In some embodiments, the samples used to acquire knowledge of or detect any of the biomarkers described herein (e.g., CD274 gene copy number alterations, such as CD274 gene copy number gains, tumor mutational burden, PD-L1 expression, and/or microsatellite instability status) comprise more than one sample (e.g., some of the biomarkers may be detected or determined in one sample, and some of the biomarkers may be detected or determined in another sample). For example, in some embodiments, CD274 gene copy number alterations, such as CD274 gene copy number gains, may be detected in one sample, and tumor mutational burden may be detected or determined in an another sample.

### F. Cancers and Methods Related Thereto

Certain aspects of the present disclosure relate to methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; monitoring progression or recurrence of a cancer in an individual; or identifying a candidate treatment for a cancer in an individual in need thereof.

In some embodiments of any of the methods provided herein, the methods comprise acquiring knowledge of or detecting in one or more samples from an individual having cancer, suspected of having cancer, being tested for cancer, or being treated for cancer, a CD274 gene copy number alteration, such as a CD274 gene copy number gain, and a high tumor mutational burden. In other embodiments, the methods comprise acquiring knowledge of or detecting in one or more samples from an individual having cancer, suspected of having cancer, being tested for cancer, or being treated for cancer, a CD274 gene copy number alteration, such as a CD274 gene copy number gain, and a high tumor mutational burden.

In some embodiments of any of the methods provided herein, detection of a CD274 gene copy number alteration, such as a CD274 gene copy number gain, and a high tumor mutational burden in one or more samples from an individual (e.g., an individual having cancer, suspected of having cancer, being tested for cancer, or being treated for cancer) identifies the individual as one who may benefit from a treatment comprising an anti-cancer therapy, such as an anti-cancer therapy provided herein, e.g., an immunotherapy.

In some embodiments, the methods comprise detecting, in one or more samples obtained from the individual at a first time point, the presence or absence of a CD274 gene copy number alteration, such as a CD274 gene copy number gain, and a high tumor mutational burden. In some embodiments, the methods further comprise detecting, in one or more samples obtained from the individual at a second time point after the first time point, the presence or absence of a CD274 gene copy number alteration, such as a CD274 gene copy number gain, and a high tumor mutational burden. In some embodiments, the methods further comprise providing an assessment of cancer progression or cancer recurrence in the individual based, at least in part, on the presence or absence of the CD274 gene copy number alteration, such as a CD274 gene copy number gain, and high tumor mutational burden in the one or more samples obtained at the first and/or second time points. In some embodiments, the presence of the CD274 gene copy number alteration, such as a CD274 gene copy number gain, and high tumor mutational burden in the one or more samples obtained from the individual at the first and/or second time points identifies the individual as having decreased risk of cancer progression or cancer recurrence when treated with a treatment comprising an anti-cancer therapy, such as an anti-cancer therapy provided herein, e.g., an immunotherapy. In some embodiments, the methods further comprise selecting a treatment, administering a treatment, adjusting a treatment, adjusting the dose of a treatment, or applying a treatment to the individual based, at least in part, on detecting the presence of the CD274 gene copy number alteration, such as a CD274 gene copy number gain, and high tumor mutational burden in the one or more samples obtained from the individual at the first and/or second time points, wherein the treatment comprises an anti-cancer therapy, such as an anti-cancer therapy provided herein, e.g., an immunotherapy.

In some embodiments, the methods comprise performing DNA sequencing on one or more samples obtained from an individual (e.g., an individual having cancer, suspected of having cancer, being tested for cancer, or being treated for cancer) to determine a sequencing mutation profile. In some embodiments, the group of genes comprises one or more cancer-related genes such as ALK, EGFR and/or CD274, or a panel of known/suspected oncogenes and/or tumor suppressors, or any combination thereof. In some embodiments, the sequencing mutation profile identifies the presence or absence of a CD274 gene copy number alteration, such as a CD274 gene copy number gain, and a high tumor mutational burden. In some embodiments, the methods further comprise identifying a candidate treatment for a cancer in an individual, based at least in part on the sequencing mutation profile. In some embodiments, the candidate treatment comprises an anti-cancer therapy, such as an anti-cancer therapy provided herein, e.g., an immunotherapy. In some embodiments, the presence of the CD274 gene copy number alteration, such as a CD274 gene copy number gain, and high tumor mutational burden in the sample(s) identifies the individual as one who may benefit from a treatment comprising an anti-cancer therapy, e.g., an anti-cancer therapy provided herein, such as an immunotherapy. In some embodiments, the presence of the CD274 gene copy number alteration, such as a CD274 gene copy number gain, and high tumor mutational burden in the sample(s) predicts the individual to have longer survival when treated with a treatment comprising an anti-cancer therapy, e.g., an immunotherapy, as compared to survival of an individual whose cancer does not comprise a CD274 gene copy number alteration, such as a CD274 gene copy number gain, and/or a high tumor mutational burden. In some embodiments, the sequencing comprises sequencing by any method known in the art or described herein, such as massively parallel sequencing (MPS) technique, whole genome sequencing (WGS), whole exome sequencing, targeted sequencing, direct sequencing, a Sanger sequencing technique, or next-generation sequencing.

In some embodiments of any of the methods provided herein, the methods further comprise generating a report comprising one or more treatment options identified for the individual based at least in part on detection of the CD274 gene copy number alteration, such as a CD274 gene copy number gain, and high tumor mutational burden in the sample(s), wherein the one or more treatment options comprise an anti-cancer therapy, such as an anti-cancer therapy provided herein, e.g., an immunotherapy. In some embodiments, the report indicates the presence or absence of the CD274 gene copy number alteration, such as a CD274 gene copy number gain, and high tumor mutational burden in the sample(s).

In some embodiments of any of the methods provided herein, responsive to acquisition of knowledge of a CD274 gene copy number alteration, such as a CD274 gene copy number gain, and high tumor mutational burden in one or more samples from an individual (e.g., an individual having cancer, suspected of having cancer, being tested for cancer, or being treated for cancer): (i) the individual is classified as a candidate to receive a treatment comprising an anti-cancer therapy, such as an anti-cancer therapy provided herein, e.g., an immunotherapy; and/or (ii) the individual is identified as likely to respond to a treatment that comprises an anti-cancer therapy, such as an anti-cancer therapy provided herein, e.g., an immunotherapy. In some embodiments, responsive to acquisition of knowledge of the CD274 gene copy number alteration, such as a CD274 gene copy number gain, and high tumor mutational burden in one or more samples from the individual, the individual is predicted to have longer survival when treated with a treatment comprising an anti-cancer therapy, such as an anti-cancer therapy provided herein, e.g., an immunotherapy, as compared to survival of an individual whose cancer does not comprise or exhibit a CD274 gene copy number alteration, such as a CD274 gene copy number gain, and/or a high tumor mutational burden.

In some embodiments, responsive to acquisition of knowledge of a CD274 gene copy number alteration, such as a CD274 gene copy number gain, and high tumor mutational burden in one or more samples from an individual (e.g., an individual having cancer, suspected of having cancer, being tested for cancer, or being treated for cancer), the methods comprise administering to the individual an effective amount of a treatment that comprises an anti-cancer therapy, such as an anti-cancer therapy provided herein, e.g., an immunotherapy.

In some embodiments of any of the methods provided herein, the methods further comprise generating a report comprising one or more treatment options identified for the individual based, at least in part, on knowledge of a CD274 gene copy number alteration, such as a CD274 gene copy number gain, and high tumor mutational burden in one or more samples from the individual, wherein the one or more treatment options comprise an anti-cancer therapy, such as an anti-cancer therapy provided herein, e.g., an immunotherapy.

In some embodiments, acquiring knowledge of a CD274 gene copy number alteration, such as a CD274 gene copy number gain, and high tumor mutational burden in one or more samples comprises detecting the CD274 gene copy number alteration, such as a CD274 gene copy number gain, and high tumor mutational burden in the sample(s). In some embodiments, the methods of the disclosure further comprise providing an assessment of the CD274 gene copy number alteration, such as a CD274 gene copy number gain, and high tumor mutational burden.

In some embodiments of any of the methods provided herein, the anti-cancer therapy is a small molecule inhibitor; an antibody; a cellular therapy; a nucleic acid; a virus-based therapy; an antibody-drug conjugate; a recombinant protein a fusion protein; a natural compound; a peptide; a PROteolysis-TArgeting Chimera (PROTAC); a treatment for cancer comprising a CD274 gene copy number alteration, such as a CD274 gene copy number gain, and/or high tumor mutational burden; a treatment for cancer being tested in a clinical trial; a treatment for cancer comprising a CD274 gene copy number alteration, such as a CD274 gene copy number gain, and/or high tumor mutational burden being tested in a clinical trial; a targeted therapy; or any combination thereof, e.g., a described in further detail below. In some embodiments, the anti-cancer therapy is an immunotherapy, such as any immunotherapy described herein or known in the art. In some embodiments, the immunotherapy is an immune checkpoint inhibitor, a cancer vaccine, a cell-based therapy, a T cell receptor (TCR)-based therapy, an adjuvant immunotherapy, a cytokine immunotherapy, or an oncolytic virus therapy. In some embodiments, the anti-cancer therapy is an immune checkpoint inhibitor, such as any immune checkpoint inhibitor described herein or known in the art. In some embodiments, the immune checkpoint inhibitor is a small molecule inhibitor; an antibody; a cellular therapy; a nucleic acid; a virus-based therapy; an antibody-drug conjugate; a recombinant protein a fusion protein; a natural compound; a peptide; a PROteolysis-TArgeting Chimera (PROTAC); a treatment for cancer comprising a CD274 gene copy number alteration, such as a CD274 gene copy number gain, and/or high tumor mutational burden; a treatment for cancer being tested in a clinical trial; a treatment for cancer comprising a CD274 gene copy number alteration, such as a CD274 gene copy number gain, and/or high tumor mutational burden being tested in a clinical trial; a targeted therapy; or any combination thereof, e.g., a described in further detail below. In some embodiments, the immunotherapy (e.g., an immune checkpoint inhibitor) is a monotherapy. In some embodiments, the immune checkpoint inhibitor is a first-line immune checkpoint inhibitor. In some embodiments, the immune checkpoint inhibitor is a second-line immune checkpoint inhibitor. In some embodiments, the immune checkpoint inhibitor is a PD-1- or a PD-L1-targeted agent. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid inhibits the expression of an immune checkpoint gene or protein. In some embodiments, the nucleic acid comprises a double-stranded RNA (dsRNA), a small interfering RNA (siRNA), or a small hairpin RNA (shRNA), e.g., as described herein.

In some embodiments of any of the methods provided herein, the methods further comprise acquiring knowledge of or detecting in one or more samples from an individual (e.g., an individual having cancer, suspected of having cancer, being tested for cancer, or being treated for cancer) the presence or absence of one or more alterations in one or more genes, such as a base substitution, a short insertion/deletion (indel), a copy number alteration, or a genomic rearrangement in one or more genes. In some embodiments, the one or more genes comprise one or more cancer-related genes such as ALK, EGFR and/or CD274, or a panel of known/suspected oncogenes and/or tumor suppressors, or any combination thereof. In some embodiments, the one or more alterations in the one or more genes are oncogenic.

In some embodiments of any of the methods provided herein, the treatment or the one or more treatment options, e.g., the immunotherapy, further comprise an additional anti-cancer therapy, e.g., an immunotherapy in combination with an additional anti-cancer therapy. In some embodiments of any of the methods provided herein, the treatment or the one or more treatment options, e.g., the immunotherapy, further comprise administering an additional anti-cancer therapy to the individual, e.g., administering an immunotherapy in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the additional anti-cancer therapy is an immunotherapy. In some embodiments, the additional anti-cancer therapy is a heat shock protein 90 inhibitor (Golding et al., Molecular cancer vol. 17,1 52, 2018; Pall, Current opinion in oncology vol. 27,2 (2015):118-24), an EGFR inhibitor (Golding et al., Molecular cancer vol. 17,1 52, 2018), a SHP2 inhibitor (Dardaei et al., Nature medicine vol. 24,4 (2018): 512-517), a MEK inhibitor (Shrestha et al., Scientific reports vol. 9,1 18842, 2019; Shrestha et al., The Journal of pharmacology and experimental therapeutics vol. 374,1 (2020):134-140), an IGF-1R inhibitor (George, Journal of hematology & oncology vol. 12,1 80, 2019), a vascular endothelial growth factor (VEGF)-targeted therapy (Makimoto et al., Acta medica Okayama vol. 74,5 (2020): 371-379; Gristina et al., Pharmaceuticals (Basel, Switzerland) vol. 13,12 474, 2020), an mTOR inhibitor (Kim et al., Anticancer research vol. 40,3 (2020): 1395-1403), or any combination thereof.

In some embodiments, the individual has been previously treated, or is being treated, for cancer with a treatment for cancer, e.g., an anti-cancer therapy described herein or any other anti-cancer therapy or treatment known in the art. In some embodiments, the individual has been previously treated, or is being treated, for cancer with an immune checkpoint inhibitor. In other embodiments, the individual has not been previously treated, or is not being treated, for cancer with a treatment for cancer, e.g., an anti-cancer therapy described herein or any other anti-cancer therapy or treatment known in the art. In certain embodiments, the individual has not been previously treated, or is not being treated, for cancer with an immune checkpoint inhibitor. In some embodiments, the cancer progressed on a prior treatment for cancer. In some embodiments, the individual, or the cancer, is immune checkpoint inhibitor naïve. In some embodiments, the individual was previously treated, or is being treated, with a VEGF-targeted anti-cancer therapy, an EGFR-targeted anti-cancer therapy, a platinum-based chemotherapy, or a single agent chemotherapy. In some embodiments, the VEGF-targeted anti-cancer therapy is an anti-VEGF chemotherapy combination treatment. In some embodiments, the EGFR-targeted anti-cancer therapy is an EGFR tyrosine kinase inhibitor.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, multiple myeloma, melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the cancer is a hematologic malignancy. In some embodiments, the cancer is a non-small cell lung cancer. In some embodiments, the cancer is a non-squamous non-small cell lung cancer.

In some embodiments, the methods further comprise detecting the presence or absence of a cancer in a sample from the individual. In some embodiments, the methods further comprise administering an effective amount of anti-cancer therapy to the individual, e.g., an anti-cancer therapy described herein, such as an immunotherapy

In some embodiments, any of the cancers described herein may comprise a CD274 gene copy number gain and high tumor mutational burden. In some embodiments, any of the cancers described herein may be assessed for a CD274 gene copy number gain and/or a high tumor mutational burden using any of the methods described herein or known in the art.

In some embodiments, any of the cancers described herein may comprise a CD274 gene copy number gain, assessed in a sample from an individual having the cancer, greater than the ploidy of the sample from the individual. In some embodiments, any of the cancers described herein may comprise a CD274 gene copy number gain, assessed in a sample from an individual having the cancer, of any of at least +1, at least +2, at least +3, at least +4, at least +5, at least +6, at least +7, at least +8, at least +9, at least +10, at least +11, at least +12, at least +13, at least +14, at least +15, at least +16, at least +17, at least +18, at least +19, at least +20, at least +21, at least +22, at least +23, at least +24, at least +25, at least +26, at least +27, at least +28, at least +29, at least +30, at least +31, at least +32, at least +33, at least +34, at least +35, at least +36, at least +37, at least +38, at least +39, at least +40, at least +41, at least +42, at least +43, at least +44, at least +45, at least +46, at least +47, at least +48, at least +49, at least +50, at least +51, at least +52, at least +53, at least +54, at least +55, at least +56, at least +57, at least +58, at least +59, at least +60, at least +61, at least +62, at least +63, at least +64, at least +65, at least +66, at least +67, at least +68, at least +69, at least +70, at least +71, at least +72, at least +73, at least +74, at least +75, at least +76, at least +77, at least +78, at least +79, at least +80, at least +81, at least +82, at least +83, at least +84, at least +85, at least +86, at least +87, at least +88, at least +89, at least +90, at least +91, at least +92, at least +93, at least +94, at least +95, at least +96, at least +97, at least +98, at least +99, at least +100, or more, as compared to the ploidy of the sample from the individual. In some embodiments, the cancer comprises a CD274 gene copy number gain of at least +2, as compared to the ploidy of the sample from the individual. In some embodiments, the ploidy of the sample from the individual is monoploid, diploid, triploid, or tetraploid. In some embodiments, the ploidy of the sample from the individual is diploid. In some embodiments, any of the cancers described herein may comprise a CD274 gene copy number gain, assessed in a sample from an individual having the cancer, greater than the ploidy of the cancer or tumor. In some embodiments, any of the cancers described herein may comprise a CD274 gene copy number gain, assessed in a sample from an individual having the cancer, of any of at least +1, at least +2, at least +3, at least +4, at least +5, at least +6, at least +7, at least +8, at least +9, at least +10, at least +11, at least +12, at least +13, at least +14, at least +15, at least +16, at least +17, at least +18, at least +19, at least +20, at least +21, at least +22, at least +23, at least +24, at least +25, at least +26, at least +27, at least +28, at least +29, at least +30, at least +31, at least +32, at least +33, at least +34, at least +35, at least +36, at least +37, at least +38, at least +39, at least +40, at least +41, at least +42, at least +43, at least +44, at least +45, at least +46, at least +47, at least +48, at least +49, at least +50, at least +51, at least +52, at least +53, at least +54, at least +55, at least +56, at least +57, at least +58, at least +59, at least +60, at least +61, at least +62, at least +63, at least +64, at least +65, at least +66, at least +67, at least +68, at least +69, at least +70, at least +71, at least +72, at least +73, at least +74, at least +75, at least +76, at least +77, at least +78, at least +79, at least +80, at least +81, at least +82, at least +83, at least +84, at least +85, at least +86, at least +87, at least +88, at least +89, at least +90, at least +91, at least +92, at least +93, at least +94, at least +95, at least +96, at least +97, at least +98, at least +99, at least +100, or more, as compared to the ploidy of the cancer or tumor. In some embodiments, the cancer comprises a CD274 gene copy number gain of at least +2, as compared to the ploidy of the cancer or tumor. In some embodiments, the ploidy of the cancer or tumor is monoploid, diploid, triploid, or tetraploid. In some embodiments, the ploidy of the cancer or tumor is diploid. In some embodiments, any of the cancers described herein may comprise a CD274 gene copy number, assessed in a sample from an individual having the cancer, of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is monoploid. In some embodiments, the cancer comprises a CD274 gene copy number of at least 2, wherein the ploidy of the sample from the individual is monoploid. In some embodiments, any of the cancers described herein may comprise a CD274 gene copy number, assessed in a sample from an individual having the cancer, of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is diploid. In some embodiments, the cancer comprises a CD274 gene copy number of at least 3, wherein the ploidy of the sample from the individual is diploid. In some embodiments, the cancer comprises a CD274 gene copy number of at least 4, wherein the ploidy of the sample from the individual is diploid. In some embodiments, any of the cancers described herein may comprise a CD274 gene copy number, assessed in a sample from an individual having the cancer, of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is triploid. In some embodiments, the cancer comprises a CD274 gene copy number of at least 4, wherein the ploidy of the sample from the individual is triploid. In some embodiments, the cancer comprises a CD274 gene copy number of at least 6, wherein the ploidy of the sample from the individual is triploid. In some embodiments, any of the cancers described herein may comprise a CD274 gene copy number, assessed in a sample from an individual having the cancer, of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is tetraploid. In some embodiments, the cancer comprises a CD274 gene copy number of at least 5, wherein the ploidy of the sample from the individual is tetraploid. In some embodiments, the cancer comprises a CD274 gene copy number of at least 8, wherein the ploidy of the sample from the individual is tetraploid. In some embodiments, any of the cancers described herein may comprise a CD274 gene copy number, assessed in a sample from an individual having the cancer, of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is monoploid. In some embodiments, the cancer comprises a CD274 gene copy number of at least 2, wherein the ploidy of the cancer or tumor is monoploid. In some embodiments, any of the cancers described herein may comprise a CD274 gene copy number, assessed in a sample from an individual having the cancer, of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is diploid. In some embodiments, the cancer comprises a CD274 gene copy number of at least 3, wherein the ploidy of the cancer or tumor is diploid. In some embodiments, the cancer comprises a CD274 gene copy number of at least 4, wherein the ploidy of the cancer or tumor is diploid. In some embodiments, any of the cancers described herein may comprise a CD274 gene copy number, assessed in a sample from an individual having the cancer, of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is triploid. In some embodiments, the cancer comprises a CD274 gene copy number of at least 4, wherein the ploidy of the cancer or tumor is triploid. In some embodiments, the cancer comprises a CD274 gene copy number of at least 6, wherein the ploidy of the cancer or tumor is triploid. In some embodiments, any of the cancers described herein may comprise a CD274 gene copy number, assessed in a sample from an individual having the cancer, of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is tetraploid. In some embodiments, the cancer comprises a CD274 gene copy number of at least 5, wherein the ploidy of the cancer or tumor is tetraploid. In some embodiments, the cancer comprises a CD274 gene copy number of at least 8, wherein the ploidy of the cancer or tumor is tetraploid. In some embodiments, the CD274 gene copy number gain is assessed using any suitable method known in the art or described herein, such as using a sequencing-based method (e.g., as described above).

In some embodiments, any of the cancers described herein may have a CD274 gene copy number gain with a ratio of CD274 to a reference or control (e.g., a reference or control gene, locus, chromosome or chromosome segment, DNA segment, centromere or centromere segment, and the like) of any of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments, any of the cancers described herein may have a CD274 gene copy number gain with a ratio of CD274 to a reference or control (e.g., a reference or control gene, locus, chromosome or chromosome segment, DNA segment, centromere or centromere segment, and the like) of at least 1.5. In some embodiments, any of the cancers described herein may have a CD274 gene copy number gain with a ratio of CD274 to a reference or control (e.g., a reference or control gene, locus, chromosome or chromosome segment, DNA segment, centromere or centromere segment, and the like) of at least 2. In some embodiments, the reference or control is a centromere or centromere segment, e.g., corresponding to any of human chromosomes 1, 2, 3, 4, 5, 6, 7, 8, 9, 10, 11, 12, 13, 14, 15, 16, 17, 18, 19, 20, 21, 22, X, Y or any combination thereof. In some embodiments, the CD274 gene copy number gain is assessed using any suitable method known in the art, such as using FISH (e.g., as described above). In some such embodiments, the ratio of CD274 to a reference or control (e.g., a reference or control gene, locus, chromosome or chromosome segment, DNA segment, centromere or centromere segment, and the like) is assessed using a FISH probe that binds to CD274 and a control FISH probe that binds to a reference or control such as a reference or control gene, locus, chromosome or chromosome segment, DNA segment, centromere or centromere segment, and the like. In some embodiments, the control FISH probe is a centromere enumeration probe, e.g., corresponding to any of human chromosomes 1, 2, 3, 4, 5, 6, 7, 8, 9, 10, 11, 12, 13, 14, 15, 16, 17, 18, 19, 20, 21, 22, X, Y or any combination thereof. In some embodiments, the control FISH probe is a CEP1, CEP2, CEP3, CEP4, CEP5, CEP6, CEP7, CEP8, CEP9, CEP10, CEP11, CEP12, CEP13, CEP14, CEP15, CEP16, CEP17, CEP18, CEP19, CEP20, CEP21, CEP22, CEPX, or CEPY FISH probe, or any combination thereof. In some embodiments, the control FISH probe is a CEP9 probe. In some embodiments, the ratio of CD274 to a reference or control is assessed based on the ratio of signal, e.g., fluorescence signal, from the FISH probe that binds to CD274 to signal, e.g., fluorescence signal, from the control FISH probe. In other embodiments, the CD274 gene copy number gain is assessed using an amplification-based method, such as PCR, e.g., qPCR or ddPCR (e.g., as described above). In some such embodiments, the ratio of CD274 to a reference or control (e.g., a reference or control gene, locus, chromosome or chromosome segment, DNA segment, centromere or centromere segment, and the like) is assessed based on the relative gene copy numbers determined by PCR (e.g., qPCR or ddPCR) of a CD274 locus (e.g., a CD274 amplicon) to a reference or control (e.g., a reference or control amplicon, e.g., a reference or control gene, locus, chromosome or chromosome segment, DNA segment, centromere or centromere segment, and the like).

In some embodiments, any of the cancers described herein may have a CD274 gene copy number gain with a ratio of CD274 assessed in a sample obtained from an individual having the cancer, to CD274 assessed in a control sample (e.g., such as from a healthy cell/tissue or individual) of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments, the ratio is at least 1.5. In some embodiments, the ratio is at least 2. In some embodiments, the CD274 gene copy number gain is assessed using any suitable method known in the art, such as using a CGH-based method (e.g., as described above). In some such embodiments, the ratio of CD274 assessed in a sample obtained from an individual having the cancer, to CD274 assessed in a control sample (e.g., such as from a healthy cell/tissue or individual) is determined based on the ratio of the amount of signal from a sample of nucleic acids from the cancer (or from the individual having the cancer) labeled with a first label, to the amount of signal from a second sample of control nucleic acids (e.g., such as from a healthy cell/tissue or individual) labeled with a second label, wherein the ratio is assessed within an array(s) at one or more positions comprising DNA fragments or oligonucleotides corresponding to chromosome 9 or portions thereof, or CD274 or portions thereof.

In some embodiments, any of the cancers described herein may have a high tumor mutational burden. In some embodiments, any of the cancers described herein may have a high tumor mutational burden with a tumor mutational burden of at least about 5 mut/Mb or at least about 10 mut/Mb. In some embodiments, any of the cancers described herein may have a high tumor mutational burden with a tumor mutational burden of at least about 10 mut/Mb. In some embodiments, any of the cancers described herein may have a tumor mutational burden of at least about 20 mut/Mb. In some embodiments, any of the cancers described herein may have a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, any of the cancers described herein may have a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, any of the cancers described herein may have a tumor mutational burden of at least about 100 mut/Mb, at least about 110 mut/Mb, at least about 120 mut/Mb, at least about 130 mut/Mb, at least about 140 mut/Mb, at least about 150 mut/Mb, or more.

In some embodiments, the cancer is a non-small cell lung cancer, such as a non-squamous non-small cell lung cancer. In some embodiments, the non-small cell lung cancer, such as a non-squamous non-small cell lung cancer, comprises a CD274 gene copy number, assessed in a sample from an individual having the non-small cell lung cancer, greater than the ploidy of the sample from the individual. In some embodiments, the non-small cell lung cancer, such as a non-squamous non-small cell lung cancer, comprises a CD274 gene copy number gain, assessed in a sample from an individual having the non-small cell lung cancer, such as a non-squamous non-small cell lung cancer, of any of at least +1, at least +2, at least +3, at least +4, at least +5, at least +6, at least +7, at least +8, at least +9, at least +10, at least +11, at least +12, at least +13, at least +14, at least +15, at least +16, at least +17, at least +18, at least +19, at least +20, at least +21, at least +22, at least +23, at least +24, at least +25, at least +26, at least +27, at least +28, at least +29, at least +30, at least +31, at least +32, at least +33, at least +34, at least +35, at least +36, at least +37, at least +38, at least +39, at least +40, at least +41, at least +42, at least +43, at least +44, at least +45, at least +46, at least +47, at least +48, at least +49, at least +50, at least +51, at least +52, at least +53, at least +54, at least +55, at least +56, at least +57, at least +58, at least +59, at least +60, at least +61, at least +62, at least +63, at least +64, at least +65, at least +66, at least +67, at least +68, at least +69, at least +70, at least +71, at least +72, at least +73, at least +74, at least +75, at least +76, at least +77, at least +78, at least +79, at least +80, at least +81, at least +82, at least +83, at least +84, at least +85, at least +86, at least +87, at least +88, at least +89, at least +90, at least +91, at least +92, at least +93, at least +94, at least +95, at least +96, at least +97, at least +98, at least +99, at least +100, or more, as compared to the ploidy of the sample from the individual. In some embodiments, the non-small cell lung cancer, such as a non-squamous non-small cell lung cancer, comprises a CD274 gene copy number gain of at least +2, as compared to the ploidy of the sample from the individual. In some embodiments, the ploidy of the sample from the individual is monoploid, diploid, triploid, or tetraploid. In some embodiments, the ploidy of the sample from the individual is diploid. In some embodiments, the non-small cell lung cancer, such as a non-squamous non-small cell lung cancer, comprises a CD274 gene copy number, assessed in a sample from an individual having the cancer, greater than the ploidy of the non-small cell lung cancer, such as a non-squamous non-small cell lung cancer. In some embodiments, the non-small cell lung cancer, such as a non-squamous non-small cell lung cancer, comprises a CD274 gene copy number gain, assessed in a sample from an individual having the cancer, of any of at least +1, at least +2, at least +3, at least +4, at least +5, at least +6, at least +7, at least +8, at least +9, at least +10, at least +11, at least +12, at least +13, at least +14, at least +15, at least +16, at least +17, at least +18, at least +19, at least +20, at least +21, at least +22, at least +23, at least +24, at least +25, at least +26, at least +27, at least +28, at least +29, at least +30, at least +31, at least +32, at least +33, at least +34, at least +35, at least +36, at least +37, at least +38, at least +39, at least +40, at least +41, at least +42, at least +43, at least +44, at least +45, at least +46, at least +47, at least +48, at least +49, at least +50, at least +51, at least +52, at least +53, at least +54, at least +55, at least +56, at least +57, at least +58, at least +59, at least +60, at least +61, at least +62, at least +63, at least +64, at least +65, at least +66, at least +67, at least +68, at least +69, at least +70, at least +71, at least +72, at least +73, at least +74, at least +75, at least +76, at least +77, at least +78, at least +79, at least +80, at least +81, at least +82, at least +83, at least +84, at least +85, at least +86, at least +87, at least +88, at least +89, at least +90, at least +91, at least +92, at least +93, at least +94, at least +95, at least +96, at least +97, at least +98, at least +99, at least +100, or more, as compared to the ploidy of the non-small cell lung cancer, such as a non-squamous non-small cell lung cancer. In some embodiments, the non-small cell lung cancer, such as a non-squamous non-small cell lung cancer, comprises a CD274 gene copy number gain of at least +2, as compared to the ploidy of the non-small cell lung cancer, such as a non-squamous non-small cell lung cancer. In some embodiments, the ploidy of the non-small cell lung cancer, such as a non-squamous non-small cell lung cancer, is monoploid, diploid, triploid, or tetraploid. In some embodiments, the ploidy of the non-small cell lung cancer, such as a non-squamous non-small cell lung cancer, is diploid. In some embodiments, the non-small cell lung cancer, such as a non-squamous non-small cell lung cancer, comprises a CD274 gene copy number, assessed in a sample from an individual having the non-small cell lung cancer, such as a non-squamous non-small cell lung cancer, of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is monoploid. In some embodiments, the non-small cell lung cancer, such as a non-squamous non-small cell lung cancer, comprises a CD274 gene copy number of at least 2, wherein the ploidy of the sample from the individual is monoploid. In some embodiments, the non-small cell lung cancer, such as a non-squamous non-small cell lung cancer, comprises a CD274 gene copy number, assessed in a sample from an individual having the non-small cell lung cancer, such as a non-squamous non-small cell lung cancer, of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is diploid. In some embodiments, the non-small cell lung cancer, such as a non-squamous non-small cell lung cancer, comprises a CD274 gene copy number of at least 3, wherein the ploidy of the sample from the individual is diploid. In some embodiments, the non-small cell lung cancer, such as a non-squamous non-small cell lung cancer, comprises a CD274 gene copy number of at least 4, wherein the ploidy of the sample from the individual is diploid. In some embodiments, the non-small cell lung cancer, such as a non-squamous non-small cell lung cancer, comprises a CD274 gene copy number, assessed in a sample from an individual having the non-small cell lung cancer, such as a non-squamous non-small cell lung cancer, of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is triploid. In some embodiments, the non-small cell lung cancer, such as a non-squamous non-small cell lung cancer, comprises a CD274 gene copy number of at least 4, wherein the ploidy of the sample from the individual is triploid. In some embodiments, the non-small cell lung cancer, such as a non-squamous non-small cell lung cancer, comprises a CD274 gene copy number of at least 6, wherein the ploidy of the sample from the individual is triploid. In some embodiments, the non-small cell lung cancer, such as a non-squamous non-small cell lung cancer, comprises a CD274 gene copy number, assessed in a sample from an individual having the non-small cell lung cancer, such as a non-squamous non-small cell lung cancer, of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is tetraploid. In some embodiments, the non-small cell lung cancer, such as a non-squamous non-small cell lung cancer, comprises a CD274 gene copy number of at least 5, wherein the ploidy of the sample from the individual is tetraploid. In some embodiments, the non-small cell lung cancer, such as a non-squamous non-small cell lung cancer, comprises a CD274 gene copy number of at least 8, wherein the ploidy of the sample from the individual is tetraploid. In some embodiments, the non-small cell lung cancer, such as a non-squamous non-small cell lung cancer, comprises a CD274 gene copy number, assessed in a sample from an individual having the non-small cell lung cancer, such as a non-squamous non-small cell lung cancer, of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the non-small cell lung cancer, such as a non-squamous non-small cell lung cancer, is monoploid. In some embodiments, the non-small cell lung cancer, such as a non-squamous non-small cell lung cancer, comprises a CD274 gene copy number of at least 2, wherein the ploidy of the non-small cell lung cancer, such as a non-squamous non-small cell lung cancer, is monoploid. In some embodiments, the non-small cell lung cancer, such as a non-squamous non-small cell lung cancer, comprises a CD274 gene copy number, assessed in a sample from an individual having the non-small cell lung cancer, such as a non-squamous non-small cell lung cancer, of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the non-small cell lung cancer, such as a non-squamous non-small cell lung cancer, is diploid. In some embodiments, the non-small cell lung cancer, such as a non-squamous non-small cell lung cancer, comprises a CD274 gene copy number of at least 3, wherein the ploidy of the non-small cell lung cancer, such as a non-squamous non-small cell lung cancer, is diploid. In some embodiments, the non-small cell lung cancer, such as a non-squamous non-small cell lung cancer, comprises a CD274 gene copy number of at least 4, wherein the ploidy of the non-small cell lung cancer, such as a non-squamous non-small cell lung cancer, is diploid. In some embodiments, the non-small cell lung cancer, such as a non-squamous non-small cell lung cancer, comprises a CD274 gene copy number, assessed in a sample from an individual having the non-small cell lung cancer, such as a non-squamous non-small cell lung cancer, of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the non-small cell lung cancer, such as a non-squamous non-small cell lung cancer, is triploid. In some embodiments, the non-small cell lung cancer, such as a non-squamous non-small cell lung cancer, comprises a CD274 gene copy number of at least 4, wherein the ploidy of the non-small cell lung cancer, such as a non-squamous non-small cell lung cancer, is triploid. In some embodiments, the non-small cell lung cancer, such as a non-squamous non-small cell lung cancer, comprises a CD274 gene copy number of at least 6, wherein the ploidy of the non-small cell lung cancer, such as a non-squamous non-small cell lung cancer, is triploid. In some embodiments, the non-small cell lung cancer, such as a non-squamous non-small cell lung cancer, comprises a CD274 gene copy number, assessed in a sample from an individual having the non-small cell lung cancer, such as a non-squamous non-small cell lung cancer, of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the non-small cell lung cancer, such as a non-squamous non-small cell lung cancer, is tetraploid. In some embodiments, the non-small cell lung cancer, such as a non-squamous non-small cell lung cancer, comprises a CD274 gene copy number of at least 5, wherein the ploidy of the non-small cell lung cancer, such as a non-squamous non-small cell lung cancer, is tetraploid. In some embodiments, the non-small cell lung cancer, such as a non-squamous non-small cell lung cancer, comprises a CD274 gene copy number of at least 8, wherein the ploidy of the non-small cell lung cancer, such as a non-squamous non-small cell lung cancer, is tetraploid. In some embodiments, the CD274 gene copy number gain is assessed using any suitable method known in the art or described herein, such as using a sequencing-based method (e.g., as described above). In some embodiments, the non-small cell lung cancer, such as a non-squamous non-small cell lung cancer, comprises a CD274 gene copy number gain with a ratio of CD274 to a reference or control (e.g., a reference or control gene, locus, chromosome or chromosome segment, DNA segment, centromere or centromere segment, and the like) of any of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments, the non-small cell lung cancer, such as a non-squamous non-small cell lung cancer, comprises a CD274 gene copy number gain with a ratio of CD274 to a reference or control (e.g., a reference or control gene, locus, chromosome or chromosome segment, DNA segment, centromere or centromere segment, and the like) of at least 1.5. In some embodiments, the non-small cell lung cancer, such as a non-squamous non-small cell lung cancer, comprises a CD274 gene copy number gain with a ratio of CD274 to a reference or control (e.g., a reference or control gene, locus, chromosome or chromosome segment, DNA segment, centromere or centromere segment, and the like) of at least 2. In some embodiments, the reference or control is a centromere or centromere segment, e.g., corresponding to any of human chromosomes 1, 2, 3, 4, 5, 6, 7, 8, 9, 10, 11, 12, 13, 14, 15, 16, 17, 18, 19, 20, 21, 22, X, Y or any combination thereof. In some embodiments, the CD274 gene copy number gain is assessed using any suitable method known in the art, such as using FISH (e.g., as described above). In some such embodiments, the ratio of CD274 to a reference or control (e.g., a reference or control gene, locus, chromosome or chromosome segment, DNA segment, centromere or centromere segment, and the like) is assessed using a FISH probe that binds to CD274 and a control FISH probe that binds to a reference or control such as a reference or control gene, locus, chromosome or chromosome segment, DNA segment, centromere or centromere segment, and the like. In some embodiments, the control FISH probe is a centromere enumeration probe, e.g., corresponding to any of human chromosomes 1, 2, 3, 4, 5, 6, 7, 8, 9, 10, 11, 12, 13, 14, 15, 16, 17, 18, 19, 20, 21, 22, X, Y or any combination thereof. In some embodiments, the control FISH probe is a CEP1, CEP2, CEP3, CEP4, CEP5, CEP6, CEP7, CEP8, CEP9, CEP10, CEP11, CEP12, CEP13, CEP14, CEP15, CEP16, CEP17, CEP18, CEP19, CEP20, CEP21, CEP22, CEPX, or CEPY FISH probe, or any combination thereof. In some embodiments, the control FISH probe is a CEP9 probe. In some embodiments, the ratio of CD274 to a reference or control is assessed based on the ratio of signal, e.g., fluorescence signal, from the FISH probe that binds to CD274 to signal, e.g., fluorescence signal, from the control FISH probe. In other embodiments, the CD274 gene copy number gain is assessed using an amplification-based method, such as PCR, e.g., qPCR or ddPCR (e.g., as described above). In some such embodiments, the ratio of CD274 to a reference or control (e.g., a reference or control gene, locus, chromosome or chromosome segment, DNA segment, centromere or centromere segment, and the like) is assessed based on the relative gene copy numbers assessed by PCR (e.g., qPCR or ddPCR) of a CD274 locus (e.g., a CD274 amplicon) to a reference or control (e.g., a reference or control amplicon, e.g., a reference or control gene, locus, chromosome or chromosome segment, DNA segment, centromere or centromere segment, and the like). In some embodiments, the non-small cell lung cancer, such as a non-squamous non-small cell lung cancer, comprises a CD274 gene copy number gain with a ratio of CD274 assessed in a sample obtained from an individual having the non-small cell lung cancer, such as a non-squamous non-small cell lung cancer, to CD274 assessed in a control sample (e.g., such as from a healthy cell/tissue or individual) of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments, the ratio is at least 1.5. In some embodiments, the ratio is at least 2. In some embodiments, the CD274 gene copy number gain is assessed using any suitable method known in the art, such as using a CGH-based method (e.g., as described above). In some such embodiments, the ratio of CD274 assessed in a sample obtained from an individual having the non-small cell lung cancer, such as a non-squamous non-small cell lung cancer, to CD274 assessed in a control sample (e.g., such as from a healthy cell/tissue or individual) is determined based on the based on the ratio of the amount of signal from a sample of nucleic acids from the non-small cell lung cancer, such as a non-squamous non-small cell lung cancer (or a sample from an individual having the non-small cell lung cancer, such as a non-squamous non-small cell lung cancer) labeled with a first label, to the amount of signal from a second sample of control nucleic acids (e.g., such as from a healthy cell/tissue or individual) labeled with a second label, wherein the ratio is determined within an array(s) at one or more positions comprising DNA fragments or oligonucleotides corresponding to chromosome 9 or portions thereof, or CD274 or portions thereof. In some embodiments, the non-small cell lung cancer, such as a non-squamous non-small cell lung cancer, comprises a high tumor mutational burden. In some embodiments, the non-small cell lung cancer, such as a non-squamous non-small cell lung cancer, comprises a high tumor mutational burden with a tumor mutational burden of at least about 10 mut/Mb. In some embodiments, the non-small cell lung cancer, such as a non-squamous non-small cell lung cancer, comprises a tumor mutational burden of at least about 20 mut/Mb. In some embodiments, the non-small cell lung cancer, such as a non-squamous non-small cell lung cancer, comprises a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the non-small cell lung cancer, such as a non-squamous non-small cell lung cancer, comprises a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, the non-small cell lung cancer, such as a non-squamous non-small cell lung cancer, comprises a tumor mutational burden of at least about 100 mut/Mb, at least about 110 mut/Mb, at least about 120 mut/Mb, at least about 130 mut/Mb, at least about 140 mut/Mb, at least about 150 mut/Mb, or more. In some embodiments, administering an anti-cancer therapy (e.g., an immunotherapy, such as an immune checkpoint inhibitor) to an individual having a non-small cell lung cancer, such as a non-squamous non-small cell lung cancer, according to the methods provided herein results in survival of the individual for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 25 months, at least about 26 months, at least about 27 months, at least about 28 months, at least about 29 months, at least about 30 months, at least about 31 months, at least about 32 months, at least about 33 months, at least about 34 months, at least about 35 months, at least about 36 months, at least about 37 months, at least about 38 months, at least about 39 months, at least about 40 months, at least about 41 months, at least about 42 months, at least about 43 months, at least about 44 months, at least about 45 months, at least about 46 months, at least about 47 months, at least about 48 months, at least about 49 months, at least about 50 months, at least about 51 months, at least about 52 months, at least about 53 months, at least about 54 months, at least about 55 months, at least about 56 months, at least about 57 months, at least about 58 months, at least about 59 months, at least about 60 months, at least about 61 months, at least about 62 months, at least about 63 months, at least about 64 months, at least about 65 months, at least about 66 months, at least about 67 months, at least about 68 months, at least about 69 months, at least about 70 months, at least about 71 months, at least about 72 months, at least about 73 months, at least about 74 months, at least about 75 months, at least about 76 months, at least about 77 months, at least about 78 months, at least about 79 months, at least about 80 months, at least about 81 months, at least about 82 months, at least about 83 months, at least about 84 months, at least about 85 months, at least about 86 months, at least about 87 months, at least about 88 months, at least about 89 months, at least about 90 months, at least about 91 months, at least about 92 months, at least about 93 months, at least about 94 months, at least about 95 months, at least about 96 months, at least about 97 months, at least about 98 months, at least about 99 months, at least about 100 months, at least about 101 months, at least about 102 months, at least about 103 months, at least about 104 months, at least about 105 months, at least about 106 months, at least about 107 months, at least about 108 months, at least about 109 months, at least about 110 months, at least about 111 months, at least about 112 months, at least about 113 months, at least about 114 months, at least about 115 months, at least about 116 months, at least about 117 months, at least about 118 months, at least about 119 months, at least about 120 months, or more, measured from the start of treatment. In some embodiments, administering an anti-cancer therapy (e.g., an immunotherapy, such as an immune checkpoint inhibitor) to a plurality of individuals having non-small cell lung cancer, such as a non-squamous non-small cell lung cancer, according to the methods provided herein results in a median overall survival of the individuals in the plurality of at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 25 months, at least about 26 months, at least about 27 months, at least about 28 months, at least about 29 months, at least about 30 months, at least about 31 months, at least about 32 months, at least about 33 months, at least about 34 months, at least about 35 months, at least about 36 months, at least about 37 months, at least about 38 months, at least about 39 months, at least about 40 months, at least about 41 months, at least about 42 months, at least about 43 months, at least about 44 months, at least about 45 months, at least about 46 months, at least about 47 months, at least about 48 months, at least about 49 months, at least about 50 months, at least about 51 months, at least about 52 months, at least about 53 months, at least about 54 months, at least about 55 months, at least about 56 months, at least about 57 months, at least about 58 months, at least about 59 months, at least about 60 months, at least about 61 months, at least about 62 months, at least about 63 months, at least about 64 months, at least about 65 months, at least about 66 months, at least about 67 months, at least about 68 months, at least about 69 months, at least about 70 months, at least about 71 months, at least about 72 months, at least about 73 months, at least about 74 months, at least about 75 months, at least about 76 months, at least about 77 months, at least about 78 months, at least about 79 months, at least about 80 months, at least about 81 months, at least about 82 months, at least about 83 months, at least about 84 months, at least about 85 months, at least about 86 months, at least about 87 months, at least about 88 months, at least about 89 months, at least about 90 months, at least about 91 months, at least about 92 months, at least about 93 months, at least about 94 months, at least about 95 months, at least about 96 months, at least about 97 months, at least about 98 months, at least about 99 months, at least about 100 months, at least about 101 months, at least about 102 months, at least about 103 months, at least about 104 months, at least about 105 months, at least about 106 months, at least about 107 months, at least about 108 months, at least about 109 months, at least about 110 months, at least about 111 months, at least about 112 months, at least about 113 months, at least about 114 months, at least about 115 months, at least about 116 months, at least about 117 months, at least about 118 months, at least about 119 months, at least about 120 months, or more, measured from the start of treatment. In some embodiments, the non-small cell lung cancer, such as a non-squamous non-small cell lung cancer, is a Stage I, Stage II, Stage III, Stage IV, or unknown stage cancer. In some embodiments, the individual having the non-small cell lung cancer, such as a non-squamous non-small cell lung cancer, has a history smoking, or does not have a history of smoking. In some embodiments, the non-small cell lung cancer, such as a non-squamous non-small cell lung cancer, is metastatic. In some embodiments, the individual having the non-small cell lung cancer, such as a non-squamous non-small cell lung cancer, has an Eastern Cooperative Oncology Group status of any of 0, 1, 2, 3, or more. In some embodiments, the non-small cell lung cancer, such as a non-squamous non-small cell lung cancer, does not comprise any oncogenic alterations in an EGFR and/or an ALK gene. In some embodiments, the non-small cell lung cancer, such as a non-squamous non-small cell lung cancer, is wild type for oncogenic alterations in an EGFR and/or an ALK gene. In some embodiments, the oncogenic alterations comprise base substitutions, insertions/deletions, copy number alterations, or rearrangements. In some embodiments, the individual has been previously treated, or is being treated, for cancer with a treatment for cancer, e.g., an anti-cancer therapy described herein or any other anti-cancer therapy or treatment known in the art. In some embodiments, the individual has not been previously treated, or is not being treated, for cancer with a treatment for cancer, e.g., an anti-cancer therapy described herein or any other anti-cancer therapy or treatment known in the art. In certain embodiments, the individual has not been previously treated, or is not being treated, for cancer with an immune checkpoint inhibitor. In some embodiments, the cancer progressed on a prior treatment for cancer. In some embodiments, the individual, or the cancer, is immune checkpoint inhibitor naïve. In some embodiments, the individual was previously treated, or is being treated, with a VEGF-targeted anti-cancer therapy, an EGFR-targeted anti-cancer therapy, a platinum-based chemotherapy, or a single agent chemotherapy. In some embodiments, the VEGF-targeted anti-cancer therapy is an anti-VEGF chemotherapy combination treatment. In some embodiments, the EGFR-targeted anti-cancer therapy is an EGFR tyrosine kinase inhibitor.

In some embodiments, overall survival (OS) refers to the length of time from the date of the start of a treatment for cancer in an individual (e.g., according to the methods provided herein), to the time of death from any cause or the time of loss of follow-up of the individual. Accordingly, in some embodiments, OS refers to the length of time from the date of the start of treatment for cancer in an individual with an anti-cancer therapy provided herein, such as an immunotherapy, to the time of death from any cause or the time of loss of follow-up of the individual. Median overall survival (e.g., of a plurality of individuals treated according to the methods of the disclosure) may be assessed using any suitable method known in the art, such as the Kaplan-Meier method, optionally in combination with a log-rank test.

In some embodiments of any of the methods provided herein, the sample is a sample described herein. In some embodiments, the sample is obtained from the individual or from the cancer. In some embodiments, the methods further comprise obtaining the sample, e.g., from the individual or from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the fusion nucleic acid molecule or polypeptide is detected in a tissue biopsy sample, in a liquid biopsy sample, or in both a tissue biopsy sample and a liquid biopsy sample, from the individual. In some embodiments, the samples used to detect/determine CD274 gene copy number alterations, such as CD274 gene copy number gains, and tumor mutational burden are the same (i.e., CD274 gene copy number alterations, such as CD274 gene copy number gains, and tumor mutational burden are detected/determined in one sample). In some embodiments of any of the methods of the disclosure, the samples used to detect/determine CD274 gene copy number alterations, such as CD274 gene copy number gains, and tumor mutational burden are different (i.e., CD274 gene copy number alterations, such as CD274 gene copy number gains, are detected/determined in one sample; and tumor mutational burden is detected/determined in another sample).

### G. Anti-Cancer Therapies

Certain aspects of the present disclosure relate to anti-cancer therapies, as well as methods for: identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; detecting the presence or absence of a cancer in an individual; monitoring progression or recurrence of a cancer in an individual; or identifying a candidate treatment for a cancer in an individual in need thereof. The present disclosure also provides uses for anti-cancer therapies (e.g., in methods of treating or delaying progression of cancer in an individual, or in methods for manufacturing a medicament for treating or delaying progression of cancer). In some instances, the methods of the disclosure can include administering a treatment comprising an anti-cancer therapy or applying a treatment comprising an anti-cancer therapy to an individual based on a generated genomic, molecular, and/or sequencing mutation profile. An anti-cancer therapy can refer to an agent or compound that is effective in the treatment of cancer cells. Examples of anti-cancer agents, compounds, or anti-cancer therapies include, but are not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some embodiments, an anti-cancer therapy of the disclosure is a small molecule inhibitor; an antibody; a cellular therapy; a nucleic acid; a virus-based therapy; an antibody-drug conjugate; a recombinant protein; a fusion protein; a natural compound; a peptide; a PROteolysis-TArgeting Chimera (PROTAC); a treatment for cancer comprising a CD274 gene copy number alteration, such as a CD274 gene copy number gain, and/or high tumor mutational burden; a treatment for cancer being tested in a clinical trial; a treatment for cancer comprising a CD274 gene copy number alteration, such as a CD274 gene copy number gain, and/or high tumor mutational burden, being tested in a clinical trial; a targeted therapy; or any combination thereof, e.g., a described in further detail below. In some embodiments, the anti-cancer therapy is an immunotherapy, such as any immunotherapy known in the art or described herein (e.g., a checkpoint inhibitor, cancer vaccine, cell-based therapy, T cell receptor (TCR)-based therapy, adjuvant immunotherapy, cytokine immunotherapy, or oncolytic virus therapy). In some embodiments, the anti-cancer therapy is an immune checkpoint inhibitor, such as any immune checkpoint inhibitor described herein or known in the art.

In some embodiments, the anti-cancer therapy comprises an immunotherapy (i.e., a cancer immunotherapy), such as a checkpoint inhibitor, cancer vaccine, cell-based therapy, T cell receptor (TCR)-based therapy, adjuvant immunotherapy, cytokine immunotherapy, and oncolytic virus therapy, as well as any combination thereof. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al, J. Clin. Invest. 95:341, 1995); naked cDNA or peptides encapsulated, e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21, 2013). In some embodiments, such cancer vaccines augment an anti-cancer response.

In some embodiments, the cancer vaccine comprises a polynucleotide that encodes a neoantigen, e.g., neoantigen(s) expressed by a cancer of the disclosure, such as a cancer in an individual. In some embodiments, the cancer vaccine comprises DNA that encodes the neoantigen(s). In some embodiments, the cancer vaccine comprises RNA that encodes the neoantigen(s). In some embodiments, the cancer vaccine comprises a polynucleotide that encodes the neoantigen(s). In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (e.g., Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (e.g., Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (e.g., Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-lh153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen expressed in a cancer of a disclosure, e.g., in an individual.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen expressed in a cancer of a disclosure, e.g., in an individual.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., expressed in a cancer of a disclosure, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., a neoantigen expressed in a cancer of a disclosure, e.g., in an individual. The TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (e.g., PROLEUKIN®; interleukin-2), interferon alfa-2a (e.g., ROFERON®-A), interferon alfa-2b (e.g., INTRON®-A), and peginterferon alfa-2b (e.g., PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-116, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-11, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-113), VTCN1 (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSFi4, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-ll,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some embodiments, the checkpoint inhibitor is a PD-1 binding antagonist/inhibitor. In some embodiments, the PD-1 binding antagonist/inhibitor is a molecule that inhibits the binding of PD-1 to its ligand binding partners. In a specific embodiment, the PD-1 ligand binding partners are PD-L1 and/or PD-L2. In some embodiments, the checkpoint inhibitor is a PD-L1 binding antagonist/inhibitor. In some embodiments, a PD-L1 binding antagonist/inhibitor is a molecule that inhibits the binding of PD-L1 to its binding ligands. In a specific embodiment, PD-L1 binding partners are PD-1 and/or B7-1. In some embodiments, the checkpoint inhibitor is a PD-L2 binding antagonist/inhibitor. In some embodiments, the PD-L2 binding antagonist/inhibitor is a molecule that inhibits the binding of PD-L2 to its ligand binding partners. In a specific embodiment, the PD-L2 binding ligand partner is PD-1. The antagonist or inhibitor may be an antibody, an antigen binding fragment thereof, an immunoadhesin, a fusion protein, or an oligopeptide. In some embodiments, the PD-1, PD-L1, or PD-L1 binding antagonist or inhibitor is a small molecule, a nucleic acid, a polypeptide (e.g., antibody), a carbohydrate, a lipid, a metal, or a toxin.

In some instances, the PD-1 binding antagonist or inhibitor is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, ENUM 388D4, cemiplimab, or dostarlimab. In other instances, the PD-1 binding antagonist or inhibitor is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist or inhibitor is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANBO11; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist or inhibitor comprises tislelizumab (BGB-A317), BGB-108, STI-A1110, AM0001, BI 754091, sintilimab (IB1I308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, e.g., Keytruda®), PF-06801591, cemiplimab (REGN-2810, REGEN2810), dostarlimab (TSR-042, ANBO11), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof, or an antibody that competes with any of the preceding.

In some embodiments, the PD-L1 binding antagonist or inhibitor is a small molecule that inhibits PD-1. In some embodiments, the PD-L1 binding antagonist or inhibitor is a small molecule that inhibits PD-L1. In some embodiments, the PD-L1 binding antagonist or inhibitor is a small molecule that inhibits PD-L1 and VISTA or PD-L1 and TIM3. In some embodiments, the PD-L1 binding antagonist or inhibitor is CA-170 (also known as AUPM-170). In some embodiments, the PD-L1 binding antagonist or inhibitor is an anti-PD-L1 antibody. In some embodiments, the anti-PD-L1 antibody can bind to a human PD-L1, for example a human PD-L1 as described above herein and/or as shown in UniProtKB/Swiss-Prot Accession No. Q9NZQ7.1, or a variant thereof. In some embodiments, the PD-L1 binding antagonist or inhibitor is a small molecule, a nucleic acid, a polypeptide (e.g., antibody), a carbohydrate, a lipid, a metal, or a toxin.

In some instances, the PD-L1 binding antagonist or inhibitor is an anti-PD-L1 antibody, for example, as described below. In some instances, the anti-PD-L1 antibody is capable of inhibiting the binding between PD-L1 and PD-1, and/or between PD-L1 and B37-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MED14736 (durvalumab), MSB0010718C (avelumab), LY3300054, STI-A1014, KN035, FAZ053, or CX-072. In some embodiments, the PD-L1 axis binding antagonist or inhibitor comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof, or an antibody that competes with any of the preceding.

In some embodiments, the checkpoint inhibitor is an antagonist or inhibitor of CTLA4. In some embodiments, the checkpoint inhibitor is a small molecule antagonist or inhibitor of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist or inhibitor is a small molecule, a nucleic acid, a polypeptide (e.g., antibody), a carbohydrate, a lipid, a metal, or a toxin. In some embodiments, the CTLA-4 antagonist or inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof, or an antibody that competes with any of the preceding.

In some embodiments, the immune checkpoint inhibitor comprises a LAG-3 antagonist or inhibitor (e.g., an antibody, an antibody conjugate, or an antigen-binding fragment thereof). In some embodiments, the LAG-3 antagonist or inhibitor comprises a small molecule, a nucleic acid, a polypeptide (e.g., an antibody), a carbohydrate, a lipid, a metal, or a toxin. In some embodiments, the LAG-3 antagonist or inhibitor comprises a small molecule. In some embodiments, the LAG-3 antagonist or inhibitor comprises a LAG-3 binding agent. In some embodiments, the LAG-3 antagonist or inhibitor comprises an antibody, an antibody conjugate, or an antigen-binding fragment thereof. In some embodiments, the LAG-3 antagonist or inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the immune checkpoint inhibitor is monovalent and/or monospecific. In some embodiments, the immune checkpoint inhibitor is multivalent and/or multispecific.

In some embodiments, an anti-cancer therapy of the disclosure (e.g., an immunotherapy) is administered in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, an anti-cancer therapy of the disclosure comprises a cyclin-dependent kinase (CDK) inhibitor, e.g., alone or in combination with an immunotherapy, such as an immune checkpoint inhibitor. In some embodiments, the CDK inhibitor inhibits CDK4. In some embodiments, the CDK inhibitor inhibits Cyclin D/CDK4. In some embodiments, the CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, an anti-cancer therapy of the disclosure comprises a murine double minute 2 homolog (MDM2) inhibitor, e.g., alone or in combination with an immunotherapy, such as an immune checkpoint inhibitor. In some embodiments, the MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, an anti-cancer therapy of the disclosure comprises (alone or in combination with an immunotherapy, such as an immune checkpoint inhibitor) one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor; an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt; a CDK inhibitor; a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, the anti-cancer therapy comprises an immunoregulatory molecule or a cytokine, e.g., alone or in combination with an immunotherapy, such as an immune checkpoint inhibitor. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groa, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, an anti-cancer therapy of the disclosure comprises a tyrosine kinase inhibitor, e.g., alone or in combination with an immunotherapy, such as an immune checkpoint inhibitor. In some embodiments, the tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, an anti-cancer therapy of the disclosure comprises a mitogen-activated protein kinase (MEK) inhibitor, e.g., alone or in combination with an immunotherapy, such as an immune checkpoint inhibitor. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, R04987655, R05126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, an anti-cancer therapy of the disclosure comprises a mammalian target of rapamycin (mTOR) inhibitor, e.g., alone or in combination with an immunotherapy, such as an immune checkpoint inhibitor. In some embodiments, the mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, an anti-cancer therapy of the disclosure comprises a PI3K inhibitor or Akt inhibitor, e.g., alone or in combination with an immunotherapy, such as an immune checkpoint inhibitor. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, an anti-cancer therapy of the disclosure comprises a hedgehog (Hh) inhibitor, e.g., alone or in combination with an immunotherapy, such as an immune checkpoint inhibitor. In some embodiments, the Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, an anti-cancer therapy of the disclosure comprises a heat shock protein (HSP) inhibitor, a MYC inhibitor, an HDAC inhibitor, an immunotherapy, a neoantigen, a vaccine, or a cellular therapy, e.g., alone or in combination with an immunotherapy, such as an immune checkpoint inhibitor.

In some embodiments, the anti-cancer therapy comprises one or more of a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor, e.g., alone or in combination with an immunotherapy, such as an immune checkpoint inhibitor.

In some embodiments, the anti-cancer therapy comprises a kinase inhibitor, e.g., alone or in combination with an immunotherapy, such as an immune checkpoint inhibitor. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described herein and/or in examples 3-39 of WO2005016894, which is incorporated herein by reference.

In some embodiments, the anti-cancer therapy comprises a heat shock protein (HSP) inhibitor, e.g., alone or in combination with an immunotherapy, such as an immune checkpoint inhibitor. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor, e.g., alone or in combination with an immunotherapy, such as an immune checkpoint inhibitor. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor, e.g., alone or in combination with an immunotherapy, such as an immune checkpoint inhibitor. In some embodiments, the HDAC inhibitor is belinostat (PXD101, e.g., Beleodaq®), SAHA (vorinostat, suberoylanilide hydroxamine, e.g., Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by L1 et al., Cold Spring Harb Perspect Med (2016) 6(10):a026831, or PX117445.

In some embodiments, the anti-cancer therapy comprises a VEGF inhibitor, e.g., alone or in combination with an immunotherapy, such as an immune checkpoint inhibitor. In some embodiments, the VEGF inhibitor is Bevacizumab (e.g., Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor, e.g., alone or in combination with an immunotherapy, such as an immune checkpoint inhibitor. In some embodiments, the integrin 03 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor, e.g., alone or in combination with an immunotherapy, such as an immune checkpoint inhibitor. In some embodiments, the αIIbβ3 integrin inhibitor is abciximab, eptifibatide (e.g., Integrilin®), or tirofiban (e.g., Aggrastat®).

In some embodiments, the anti-cancer therapy comprises an mTOR inhibitor, e.g., alone or in combination with an immunotherapy, such as an immune checkpoint inhibitor. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a statin or a statin-based agent, e.g., alone or in combination with an immunotherapy, such as an immune checkpoint inhibitor. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor, e.g., alone or in combination with an immunotherapy, such as an immune checkpoint inhibitor. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises an EGFR inhibitor, e.g., alone or in combination with an immunotherapy, such as an immune checkpoint inhibitor. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (ACO0010), rociletinib (CO-1686), BMS-690514, YH5448, PF-06747775, ASP8273, PF299804, AP26113, necitumumab (e.g., Portrazza®), or erlotinib. In some embodiments, the EGFR inhibitor is gefitinib or cetuximab.

In some embodiments, the anti-cancer therapy comprises a nucleic acid molecule, such as a dsRNA, an siRNA, or an shRNA, e.g., alone or in combination with an immunotherapy, such as an immune checkpoint inhibitor. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

In some embodiments, the anti-cancer therapy comprises a chemotherapy, e.g., alone or in combination with an immunotherapy, such as an immune checkpoint inhibitor. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor, e.g., alone or in combination with an immunotherapy, such as an immune checkpoint inhibitor. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR2, FGFR3, FGFR4, CSF1R, c-Met, ROS1, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent, e.g., alone or in combination with an immunotherapy, such as an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy, e.g., alone or in combination with an immunotherapy, such as an immune checkpoint inhibitor. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer, e.g., alone or in combination with an immunotherapy, such as an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent, e.g., alone or in combination with an immunotherapy, such as an immune checkpoint inhibitor. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-cB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (e.g., Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (e.g., ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (e.g., Humira®), certolizumab pegol (e.g., Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, e.g., Ala-Cort®, Hydrocort Acetate®, hydrocortone phosphate Lanacort®, Solu-Cortef®), decadron (dexamethasone, dexamethasone acetate, dexamethasone sodium phosphate, e.g., Dexasone®, Diodex®, Hexadrol®, Maxidex®), methylprednisolone (6-methylprednisolone, methylprednisolone acetate, methylprednisolone sodium succinate, e.g., Duralone®, Medralone®, Medrol®, M-Prednisol®, Solu-Medrol®), prednisolone (e.g., Delta-Cortef®, ORAPRED®, Pediapred®, Prezone®), and prednisone (e.g., Deltasone®, Liquid Pred®, Meticorten®, Orasone®), and bisphosphonates (e.g., pamidronate (Aredia®), and zoledronic acid (e.g., Zometac®).

In some embodiments, the anti-cancer therapy comprises an anti-hormonal agent, e.g., alone or in combination with an immunotherapy, such as an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent, e.g., alone or in combination with an immunotherapy, such as an immune checkpoint inhibitor. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (e.g., GEMZAR®), 5-fluorouracil (5-FU), capecitabine (e.g., XELODA™), 6-mercaptopurine, methotrexate, 6-thioguanine, pemetrexed, raltitrexed, arabinosylcytosine ARA-C cytarabine (e.g., CYTOSAR-U®), dacarbazine (DTIC-DOMED), azocytosine, deoxycytosine, pyridmidene, fludarabine (e.g., FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent, e.g., alone or in combination with an immunotherapy, such as an immune checkpoint inhibitor. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some embodiments, acquiring knowledge of or detecting the presence of a CD274 gene copy number gain and a high TMB in cancer or in a sample from an individual (e.g., an individual having a cancer) may be a predictor of non-response to anti-cancer therapies such as chemotherapies or chemotherapeutic agents, targeted therapies, alkylating agents, antimetabolites, natural products, hormones, radiation therapy, or a therapy configured to target a defect in a specific cell signaling pathway, e.g., a defect in a DNA mismatch repair (MMR) pathway. Accordingly, in some embodiments, acquiring knowledge of or detecting the presence of a CD274 gene copy number gain and a high TMB in cancer or in a sample from an individual (e.g., an individual having a cancer) may identify an individual having the cancer as one who may not benefit from an anti-cancer therapy (such as chemotherapies or chemotherapeutic agents, targeted therapies, alkylating agents, antimetabolites, natural products, hormones, radiation therapy, or a therapy configured to target a defect in a specific cell signaling pathway), and optionally as one who may benefit from a treatment comprising an immunotherapy, such an immune checkpoint inhibitor.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an anti-cancer therapy of the disclosure is administered as a monotherapy. In some embodiments, the anti-cancer therapy is administered in combination with one or more additional anti-cancer therapies or treatments, e.g., as described herein. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy. In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

### H. Reporting

In some embodiments, the methods provided herein comprise generating a report, and/or providing a report to party.

In some embodiments, a report according to the present disclosure comprises information about one or more of: a CD274 gene copy number alteration, such as a CD274 gene copy number gain, and/or a high tumor mutational burden; a cancer of the disclosure, e.g., comprising a CD274 gene copy number alteration, such as a CD274 gene copy number gain, and/or a high tumor mutational burden; or a treatment, a therapy, or one or more treatment options for an individual having a cancer, such as a cancer of the disclosure (e.g., an immunotherapy, such as immune checkpoint inhibitor).

In some embodiments, a report according to the present disclosure comprises information about the presence or absence of a CD274 gene copy number alteration, such as a CD274 gene copy number gain, and/or a high tumor mutational burden in one or more samples obtained from an individual, such as an individual having a cancer, e.g., a cancer provided herein. In one embodiment, a report according to the present disclosure indicates that a CD274 gene copy number alteration, such as a CD274 gene copy number gain, and/or a high tumor mutational burden are present in one or more samples obtained from the individual. In one embodiment, a report according to the present disclosure indicates that a CD274 gene copy number alteration, such as a CD274 gene copy number gain, and/or a high tumor mutational burden is not present in one or more samples obtained from the individual. In one embodiment, a report according to the present disclosure indicates that a CD274 gene copy number alteration, such as a CD274 gene copy number gain, and/or a high tumor mutational burden has been detected in one or more samples obtained from the individual. In one embodiment, a report according to the present disclosure indicates that a CD274 gene copy number alteration, such as a CD274 gene copy number gain, and/or a high tumor mutational burden has not been detected in one or more samples obtained from the individual. In some embodiments, the report comprises an identifier for the individual from which the sample(s) was obtained.

In some embodiments, the report includes information on the role of a CD274 gene copy number alteration, such as a CD274 gene copy number gain, and/or a high tumor mutational burden in disease, such as in cancer. Such information can include one or more of: information on prognosis of a cancer, such as a cancer provided herein, e.g., comprising a CD274 gene copy number alteration, such as a CD274 gene copy number gain, and/or a high tumor mutational burden; information on resistance of a cancer, such as a cancer provided herein (e.g., comprising a CD274 gene copy number alteration, such as a CD274 gene copy number gain, and/or a high tumor mutational burden) to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein (e.g., comprising a CD274 gene copy number alteration, such as a CD274 gene copy number gain, and/or a high tumor mutational burden) and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining one or more samples, such as sample(s) described herein, from an individual, e.g., an individual having a cancer, such as a cancer provided herein; detecting a CD274 gene copy number alteration, such as a CD274 gene copy number gain, and/or a high tumor mutational burden in the sample(s), or acquiring knowledge of the presence of a CD274 gene copy number alteration, such as a CD274 gene copy number gain, and/or a high tumor mutational burden in the sample(s); and generating a report. In some embodiments, a report generated according to the methods provided herein comprises one or more of: information about the presence or absence of a CD274 gene copy number alteration, such as a CD274 gene copy number gain, and/or a high tumor mutational burden in the sample(s); an identifier for the individual from which the sample(s) was obtained; information on the role of the CD274 gene copy number alteration, such as a CD274 gene copy number gain, and/or a high tumor mutational burden, in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient having, suspected of having, or being tested for a cancer, such as a cancer provided herein, e.g., comprising a CD274 gene copy number alteration, such as a CD274 gene copy number gain, and/or a high tumor mutational burden), or to an individual or entity other than the individual or patient, such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from the individual. In some embodiments, the report is provided or delivered to an individual or entity within any of about 1 day or more, about 7 days or more, about 14 days or more, about 21 days or more, about 30 days or more, about 45 days or more, or about 60 days or more from detecting a CD274 gene copy number alteration, such as a CD274 gene copy number gain, and/or a high tumor mutational burden in one or more samples obtained from the individual. In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a CD274 gene copy number alteration, such as a CD274 gene copy number gain, and/or a high tumor mutational burden in one or more samples obtained from the individual. In some instances, all or a portion of the report may be displayed in a graphical user interface of an online or web-based healthcare portal.

### I. Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform a method according to any of the embodiments described herein.

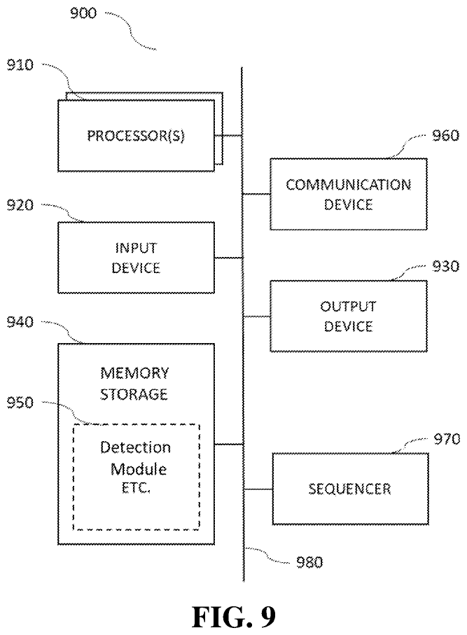

FIG. 9 illustrates an example of a computing device or system in accordance with one embodiment. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting a CD274 gene copy number alteration, such as a CD274 gene copy number gain, and/or a high tumor mutational burden. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting a CD274 gene copy number alteration, such as a CD274 gene copy number gain, and/or a high tumor mutational burden (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

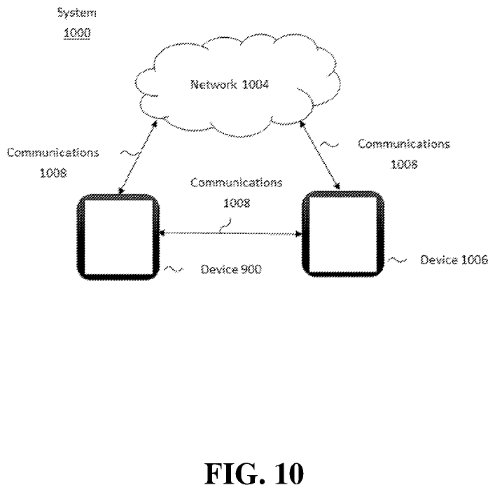

Device 900 may be connected to a network (e.g., network 1004, as shown in FIG. 10 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

FIG. 10 illustrates an example of a computing system in accordance with one embodiment. In computing system 1000, device 900 (e.g., as described above and illustrated in FIG. 9) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

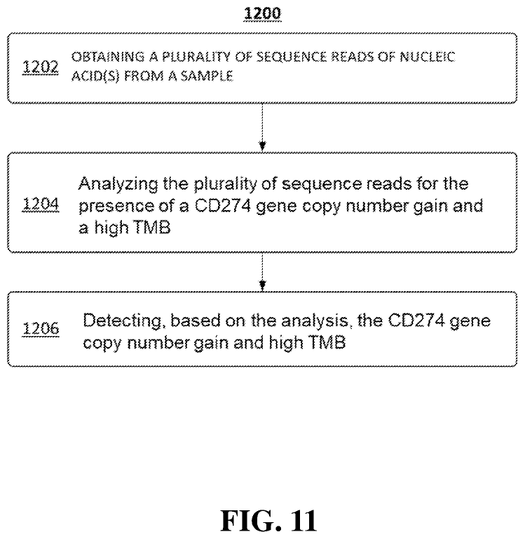

FIG. 11 illustrates an exemplary process 1200 for detecting a CD274 gene copy number alteration, such as a CD274 gene copy number gain, and/or a high tumor mutational burden in one or more samples, in accordance with some embodiments of the present disclosure. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In some embodiments, the executed steps can be executed across many systems, e.g., in a cloud environment. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of sequence reads of one or more nucleic acid molecules is obtained, wherein the one or more nucleic acid molecules are derived from one or more samples obtained from an individual, e.g., as described herein. In some embodiments, the one or more samples are obtained from an individual having, suspected of having, or being tested for a cancer, such as a cancer described herein. In some embodiments, the sequence reads are obtained using a sequencer, e.g., as described herein or otherwise known in the art. In some embodiments, the nucleic acid molecules comprise one or more nucleic acid molecules corresponding to CD274 and/or one or more genes such as one or more cancer-related genes, ALK, EGFR, or a panel of known/suspected oncogenes and/or tumor suppressors, or any combination thereof, or fragments thereof. Optionally, prior to obtaining the sequence reads, the sample(s) are purified, enriched (e.g., for nucleic acid(s) corresponding to: CD274 and/or one or more genes such as one or more cancer-related genes, ALK, EGFR, or a panel of known/suspected oncogenes and/or tumor suppressors, or any combination thereof, or fragments thereof), and/or subjected to PCR amplification. At block 1204, an exemplary system (e.g., one or more electronic devices) analyzes the plurality of sequence reads for the presence of a CD274 gene copy number alteration, such as a CD274 gene copy number gain, and/or a high tumor mutational burden. At block 1206, the system detects (e.g., based on the analysis) a CD274 gene copy number alteration, such as a CD274 gene copy number gain, and/or a high tumor mutational burden, in the one or more samples. In some embodiments, the analyzing step comprises: (a) generating a copy number model based on the plurality of sequence reads; and (b) determining a CD274 gene copy number based on the copy number model. In some embodiments, the copy number model is generated by: (a) aligning the plurality of sequence reads against a reference genome; (b) normalizing sequence coverage distribution of the aligned plurality of sequence reads against a control sample; and (c) generating segmentation data for the normalized plurality of sequence reads. In some embodiments, the analyzing step comprises: determining coverage ratio data, allele fraction data, and segmentation data for one or more gene loci within one or more subgenomic intervals of the plurality of sequence reads, wherein the one or more gene loci comprise CD274; identifying a plurality of segments based on the segmentation data; determining copy numbers for the plurality of segments based on the coverage ratio data, the allele fraction data, the segmentation data, and a copy number model; detecting the presence or absence of a CD274 copy number gain based on a copy number of a segment of the plurality of segments corresponding to CD274. In some embodiments, a CD274 copy number gain is detected when the copy number for the segment of the plurality of segments corresponding to CD274 is greater than or equal to ploidy of the sample. In some embodiments, a CD274 copy number gain is detected when the copy number for the segment of the plurality of segments corresponding to CD274 is greater than ploidy of the sample. In some embodiments, the coverage ratio data is determined by aligning a plurality of sequence reads from the sample and from a control sample to a reference genome, and determining a number of sequence reads that overlap each of the one or more gene loci within the one or more subgenomic intervals in the sample and in the control sample. In some embodiments, the control sample is a paired normal sample, a process-matched control sample, or a panel of normal control sample. In some embodiments, the copy number model: (a) predicts a copy number for CD274 based on coverage ratio data and allele fraction data; (b) predicts a sample purity and ploidy for the sample; (c) outputs segmentation data; and any combination thereof. In some embodiments, the segmentation data is generated by aligning a plurality of sequence reads from the sample to a reference genome, and processing the aligned sequence read data, coverage ratio data, and allele fraction data to determine a number of segments required to account for the aligned sequence read data. In some embodiments, each segment has the same copy number. In some embodiments, the method comprises processing the aligned sequence read data, coverage ratio data, and allele fraction data using a pruned exact linear time (PELT) method to determine a number of segments required to account for the aligned sequence read data, wherein each segment has a same copy number. In some embodiments, the reference genome is a human genome. In some embodiments, the segmentation data is generated using a circular binary segmentation (CBS) method or any other suitable method. In some embodiments, the CD274 gene copy number is determined based on a summary statistic of the copy number of all genomic segments overlapping a CD274 gene, e.g., the mean, median, maximum or mode of the copy number of all genomic segments overlapping a CD274 gene. In some embodiments, the copy number model is a genome-wide copy number model. In some embodiments, the segmentation comprises whole-genome segmentation. In some embodiments, each segment has an equal copy number. In some embodiments, the method further comprises calling a CD274 copy number gain based on a copy number for the segment of the plurality of segments corresponding to CD274 that is greater than or equal to ploidy of the sample, or based on detecting the presence or absence of a CD274 copy number gain. In some embodiments, the method further comprises calling a CD274 copy number gain based on a copy number for the segment of the plurality of segments corresponding to CD274 is greater than ploidy of the sample.

In some embodiments of any of the methods, systems, devices, non-transitory computer readable storage media, or processes of the disclosure, detection of a CD274 gene copy number gain, and a high tumor mutational burden, in a cancer (e.g., in one or more samples) identifies the individual having the cancer as one who may benefit from a treatment comprising an anti-cancer therapy, e.g., an anti-cancer therapy provided herein, such as an immunotherapy, e.g., an immune checkpoint inhibitor. In some embodiments of any of the methods, systems, devices, non-transitory computer readable storage media, or processes of the disclosure, detection of a CD274 gene copy number gain, and a high tumor mutational burden, in a cancer (e.g., in one or more samples) predicts the individual having the cancer to have longer survival when treated with a treatment comprising an anti-cancer therapy, e.g., an immunotherapy, such as an immune checkpoint inhibitor, as compared to survival of an individual whose cancer does not comprise a CD274 gene copy number gain, and/or a high tumor mutational burden. In some embodiments of any of the methods, systems, devices, non-transitory computer readable storage media, or processes of the disclosure, detection of a CD274 gene copy number gain, and a high tumor mutational burden, in a cancer (e.g., in one or more samples) identifies the individual having the cancer to be a candidate to receive a treatment comprising an anti-cancer therapy, e.g., an immunotherapy, such as an immune checkpoint inhibitor. In some embodiments of any of the methods, systems, devices, non-transitory computer readable storage media, or processes of the disclosure, detection of a CD274 gene copy number gain, and a high tumor mutational burden, in a cancer (e.g., in one or more samples) identifies the individual having the cancer as likely to respond (e.g., to have a therapeutic response) to a treatment comprising an anti-cancer therapy, e.g., an immunotherapy, such as an immune checkpoint inhibitor. In some embodiments of any of the methods, systems, devices, non-transitory computer readable storage media, or processes of the disclosure, detection of a CD274 gene copy number gain, and a high tumor mutational burden, in a cancer (e.g., in one or more samples) identifies the individual having the cancer as likely to have an improved response when treated with a treatment comprising an anti-cancer therapy, e.g., an immunotherapy, such as an immune checkpoint inhibitor, as compared to an individual whose cancer does not comprise a CD274 gene copy number gain, and/or a high tumor mutational burden.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the samples used to acquire knowledge of or detect any of the biomarkers described herein (e.g., CD274 gene copy number alterations, such as CD274 gene copy number gains, tumor mutational burden, PD-L1 expression, and/microsatellite instability status) are the same sample (i.e., one or more, or all, of CD274 gene copy number alterations, such as CD274 gene copy number gains, tumor mutational burden, PD-L1 expression, and/microsatellite instability status are detected or determined in one sample). In some embodiments, the samples used to acquire knowledge of or detect any of the biomarkers described herein (e.g., CD274 gene copy number alterations, such as CD274 gene copy number gains, tumor mutational burden, PD-L1 expression, and/microsatellite instability status) comprise more than one sample (e.g., some of the biomarkers may be detected or determined in one sample, and some of the biomarkers may be detected or determined in another sample). For example, in some embodiments, CD274 gene copy number alterations, such as CD274 gene copy number gains, may be detected in one sample, and tumor mutational burden may be detected or determined in an another sample. In some embodiments, the samples used to detect/determine CD274 gene copy number alterations, such as CD274 gene copy number gains, and tumor mutational burden are the same (i.e., CD274 gene copy number alterations, such as CD274 gene copy number gains, and tumor mutational burden are detected/determined in one sample). In some embodiments, the samples used to detect/determine CD274 gene copy number alterations, such as CD274 gene copy number gains, and tumor mutational burden are different (i.e., CD274 gene copy number alterations, such as CD274 gene copy number gains, are detected/determined in one sample; and tumor mutational burden is detected/determined in another sample).

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, multiple myeloma, melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the cancer is a hematologic malignancy. In some embodiments of any of the methods, systems, devices, non-transitory computer readable storage media, or processes of the disclosure, the cancer is a non-small cell lung cancer. In some embodiments of any of the methods, systems, devices, non-transitory computer readable storage media, or processes of the disclosure, the cancer is a non-squamous non-small cell lung cancer.

In some embodiments of any of the methods, systems, devices, non-transitory computer readable storage media, or processes of the disclosure, a CD274 gene copy number gain and high tumor mutational burden may be detected, assessed or determined in any of the cancers described herein.

In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, greater than the ploidy of the sample from the individual. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number gain, assessed in a sample from an individual having the cancer, of any of at least +1, at least +2, at least +3, at least +4, at least +5, at least +6, at least +7, at least +8, at least +9, at least +10, at least +11, at least +12, at least +13, at least +14, at least +15, at least +16, at least +17, at least +18, at least +19, at least +20, at least +21, at least +22, at least +23, at least +24, at least +25, at least +26, at least +27, at least +28, at least +29, at least +30, at least +31, at least +32, at least +33, at least +34, at least +35, at least +36, at least +37, at least +38, at least +39, at least +40, at least +41, at least +42, at least +43, at least +44, at least +45, at least +46, at least +47, at least +48, at least +49, at least +50, at least +51, at least +52, at least +53, at least +54, at least +55, at least +56, at least +57, at least +58, at least +59, at least +60, at least +61, at least +62, at least +63, at least +64, at least +65, at least +66, at least +67, at least +68, at least +69, at least +70, at least +71, at least +72, at least +73, at least +74, at least +75, at least +76, at least +77, at least +78, at least +79, at least +80, at least +81, at least +82, at least +83, at least +84, at least +85, at least +86, at least +87, at least +88, at least +89, at least +90, at least +91, at least +92, at least +93, at least +94, at least +95, at least +96, at least +97, at least +98, at least +99, at least +100, or more, as compared to the ploidy of the sample from the individual. In some embodiments, the cancer comprises a CD274 gene copy number gain of at least +2, as compared to the ploidy of the sample from the individual. In some embodiments, the ploidy of the sample from the individual is monoploid, diploid, triploid, or tetraploid. In some embodiments, the ploidy of the sample from the individual is diploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, greater than the ploidy of the cancer or tumor. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number gain, assessed in a sample from an individual having the cancer, of any of at least +1, at least +2, at least +3, at least +4, at least +5, at least +6, at least +7, at least +8, at least +9, at least +10, at least +11, at least +12, at least +13, at least +14, at least +15, at least +16, at least +17, at least +18, at least +19, at least +20, at least +21, at least +22, at least +23, at least +24, at least +25, at least +26, at least +27, at least +28, at least +29, at least +30, at least +31, at least +32, at least +33, at least +34, at least +35, at least +36, at least +37, at least +38, at least +39, at least +40, at least +41, at least +42, at least +43, at least +44, at least +45, at least +46, at least +47, at least +48, at least +49, at least +50, at least +51, at least +52, at least +53, at least +54, at least +55, at least +56, at least +57, at least +58, at least +59, at least +60, at least +61, at least +62, at least +63, at least +64, at least +65, at least +66, at least +67, at least +68, at least +69, at least +70, at least +71, at least +72, at least +73, at least +74, at least +75, at least +76, at least +77, at least +78, at least +79, at least +80, at least +81, at least +82, at least +83, at least +84, at least +85, at least +86, at least +87, at least +88, at least +89, at least +90, at least +91, at least +92, at least +93, at least +94, at least +95, at least +96, at least +97, at least +98, at least +99, at least +100, or more, as compared to the ploidy of the cancer or tumor. In some embodiments, the cancer comprises a CD274 gene copy number gain of at least +2, as compared to the ploidy of the cancer or tumor. In some embodiments, the ploidy of the cancer or tumor is monoploid, diploid, triploid, or tetraploid. In some embodiments, the ploidy of the cancer or tumor is diploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is monoploid. In some embodiments, the cancer comprises a CD274 gene copy number of at least 2, wherein the ploidy of the sample from the individual is monoploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is diploid. In some embodiments, the cancer comprises a CD274 gene copy number of at least 3, wherein the ploidy of the sample from the individual is diploid. In some embodiments, the cancer comprises a CD274 gene copy number of at least 4, wherein the ploidy of the sample from the individual is diploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is triploid. In some embodiments, the cancer comprises a CD274 gene copy number of at least 4, wherein the ploidy of the sample from the individual is triploid. In some embodiments, the cancer comprises a CD274 gene copy number of at least 6, wherein the ploidy of the sample from the individual is triploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the sample from the individual is tetraploid. In some embodiments, the cancer comprises a CD274 gene copy number of at least 5, wherein the ploidy of the sample from the individual is tetraploid. In some embodiments, the cancer comprises a CD274 gene copy number of at least 8, wherein the ploidy of the sample from the individual is tetraploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is monoploid. In some embodiments, the cancer comprises a CD274 gene copy number of at least 2, wherein the ploidy of the cancer or tumor is monoploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is diploid. In some embodiments, the cancer comprises a CD274 gene copy number of at least 3, wherein the ploidy of the cancer or tumor is diploid. In some embodiments, the cancer comprises a CD274 gene copy number of at least 4, wherein the ploidy of the cancer or tumor is diploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is triploid. In some embodiments, the cancer comprises a CD274 gene copy number of at least 4, wherein the ploidy of the cancer or tumor is triploid. In some embodiments, the cancer comprises a CD274 gene copy number of at least 6, wherein the ploidy of the cancer or tumor is triploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number, assessed in a sample from an individual having the cancer, of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more, wherein the ploidy of the cancer or tumor is tetraploid. In some embodiments, the cancer comprises a CD274 gene copy number of at least 5, wherein the ploidy of the cancer or tumor is tetraploid. In some embodiments, the cancer comprises a CD274 gene copy number of at least 8, wherein the ploidy of the cancer or tumor is tetraploid. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number gain with a ratio of CD274 to a reference or control (e.g., a reference or control gene, locus, chromosome or chromosome segment, DNA segment, centromere or centromere segment, and the like) of any of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number gain with a ratio of CD274 to a reference or control (e.g., a reference or control gene, locus, chromosome or chromosome segment, DNA segment, centromere or centromere segment, and the like) of at least 1.5. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number gain with a ratio of CD274 to a reference or control (e.g., a reference or control gene, locus, chromosome or chromosome segment, DNA segment, centromere or centromere segment, and the like) of at least 2. In some embodiments of any of the methods, systems, devices, non-transitory computer readable storage media, or processes of the disclosure, a CD274 gene copy number gain may be detected, assessed or determined in any of the cancers described herein, wherein the cancer comprises a CD274 gene copy number gain with a ratio of CD274 assessed in a sample obtained from an individual having the cancer, to CD274 assessed in a control sample (e.g., such as from a healthy cell/tissue or individual) of at least 1.5, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, or more. In some embodiments, the ratio is at least 1.5. In some embodiments, the ratio is at least 2. In some embodiments of any of the methods, systems, devices, non-transitory computer readable storage media, or processes of the disclosure, a high tumor mutational burden may be detected, assessed or determined in any of the cancers described herein, wherein the cancer comprises a high tumor mutational burden of at least about 5 mut/Mb, or at least about 10 mut/Mb. In some embodiments of any of the methods, systems, devices, non-transitory computer readable storage media, or processes of the disclosure, a high tumor mutational burden may be detected, assessed or determined in any of the cancers described herein, wherein the cancer comprises a tumor mutational burden of at least about 20 mut/Mb. In some embodiments of any of the methods, systems, devices, non-transitory computer readable storage media, or processes of the disclosure, a high tumor mutational burden may be detected, assessed or determined in any of the cancers described herein, wherein the cancer comprises a tumor mutational burden of any of between about 5 mut/Mb and about 10 mut/Mb, between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments of any of the methods, systems, devices, non-transitory computer readable storage media, or processes of the disclosure, a high tumor mutational burden may be detected, assessed or determined in any of the cancers described herein, wherein the cancer comprises a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments of any of the methods, systems, devices, non-transitory computer readable storage media, or processes of the disclosure, a high tumor mutational burden may be detected, assessed or determined in any of the cancers described herein, wherein the cancer comprises a tumor mutational burden of at least about 100 mut/Mb, at least about 110 mut/Mb, at least about 120 mut/Mb, at least about 130 mut/Mb, at least about 140 mut/Mb, at least about 150 mut/Mb, or more. In some embodiments of any of the methods, systems, devices, non-transitory computer readable storage media, or processes of the disclosure, the cancer is a non-small cell lung cancer, such as a non-squamous non-small cell lung cancer. In some embodiments of any of the methods, systems, devices, non-transitory computer readable storage media, or processes of the disclosure, the cancer is a non-squamous non-small cell lung cancer.

In some embodiments of any of the methods, systems, devices, non-transitory computer readable storage media, or processes of the disclosure, the individual is administered a treatment based at least in part on detection of a CD274 gene copy number alteration, such as a CD274 gene copy number gain, and a high tumor mutational burden in the cancer in an individual (e.g., in one or more samples from the individual). In some embodiments, the treatment is an anti-cancer therapy known in the art or described herein, e.g., an immunotherapy, such as an immune checkpoint inhibitor.

In some embodiments of any of the methods, systems, devices, non-transitory computer readable storage media, or processes of the disclosure, the disclosed methods for determining the presence or absence of a CD274 gene copy number alteration, such as a CD274 gene copy number gain, and/or a high tumor mutational burden may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of a CD274 gene copy number alteration, such as a CD274 gene copy number gain, and/or a high tumor mutational burden as part of a genomic profiling process can improve the validity of, e.g., disease detection calls, made on the basis of the genomic profile by, for example, independently confirming the presence of the CD274 gene copy number alteration, such as a CD274 gene copy number gain, and/or a high tumor mutational burden in a given patient sample(s). In some embodiments, the comprehensive genomic profiling includes detecting, determining, or acquiring information on the presence of genes (or variant sequences thereof), copy number variations, epigenetic traits, proteins (or modifications thereof), and/or other biomarkers in an individual's genome and/or proteome, as well as the individual's corresponding phenotypic traits and the interaction between genetic or genomic traits, phenotypic traits, and environmental factors. In some instances, the comprehensive genomic profiling includes results from a nucleic acid sequencing-based test, a gene expression profiling test, a cancer hotspot panel test, a DNA methylation test, a DNA fragmentation test, an RNA fragmentation test, or any combination thereof.

In some embodiments of any of the methods, systems, devices, non-transitory computer readable storage media, or processes of the disclosure, a molecular profile is generated for the individual or the sample, based, at least in part, on detecting a CD274 gene copy number alteration, such as a CD274 gene copy number gain, and/or a high tumor mutational. In some instances, the molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. In some instances, the molecular profile may comprise results from a comprehensive genomic profiling (CGP) test (e.g., as describe above), a nucleic acid sequencing-based test, a gene expression profiling test, a cancer hotspot panel test, a DNA methylation test, a DNA fragmentation test, an RNA fragmentation test, or any combination thereof. In some embodiments, the molecular profile further comprises/indicates/comprises information on presence or absence of mutations in one or more additional genes, e.g., a panel of known/suspected oncogenes and/or tumor suppressors. In some embodiments, the one or more additional genes comprise one or more cancer-related genes, ALK, EGFR, CD274, or any combination thereof. In some embodiments, the molecular profile is obtained from a genomic profiling assay (such as a cancer- or tumor-related genomic profiling assay), e.g., as obtained using any of the sequencing methodologies described herein. In some embodiments, the molecular profile includes information from whole-genome or whole-exome sequencing. In some embodiments, the molecular profile includes information from targeted sequencing. In some embodiments, the molecular profile includes information from NGS. In some embodiments, the molecular profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement) of one or more genes, e.g., a panel of known/suspected oncogenes and/or tumor suppressors, one or more cancer-related genes, ALK, EGFR, CD274, or any combination thereof. In some embodiments, the individual is administered a treatment based at least in part on the molecular profile. In some embodiments, the treatment is an anti-cancer therapy known in the art or described herein, e.g., an immunotherapy, such as an immune checkpoint inhibitor.

In some embodiments of any of the methods, systems, devices, non-transitory computer readable storage media, or processes of the disclosure, a report is generated, e.g., as described in further detail above. In some embodiments, the report comprises/indicates/comprises information on the presence or absence of a CD274 gene copy number alteration, such as a CD274 gene copy number gain, and a high tumor mutational burden in the cancer in an individual (e.g., in one or more samples from the individual). In some embodiments, the report comprises/indicates/comprises information on results of a genomic profiling process of a cancer in an individual (e.g., in one or more samples from the individual), e.g., as described above. In some embodiments, the report comprises/indicates/comprises information on results of comprehensive genomic profiling of a cancer in an individual (e.g., in one or more samples from the individual), e.g., as described above. In some embodiments, the report comprises/indicates/comprises information on a molecular profile generated for the individual or the sample, e.g., as described above. In some embodiments, the report comprises/indicates/comprises information on a treatment or one or more treatment options selected or identified for the individual, based, at least in part, on the presence of a CD274 gene copy number alteration, such as a CD274 gene copy number gain, and a high tumor mutational burden in the cancer in an individual (e.g., in one or more samples from the individual), and optionally based on results of a genomic profiling process, comprehensive genomic profiling, and/or a molecular profile generated for the individual or a sample, e.g., as described above. In some embodiments, the treatment or one or more treatment options comprise an anti-cancer therapy known in the art or described herein, e.g., an immunotherapy, such as an immune checkpoint inhibitor. In some embodiments, the report is provided or transmitted to the individual, a caregiver, a healthcare provider, a physician, an oncologist, an electronic medical record system, a hospital, a clinic, a third-party payer, an insurance company, or a government office, e.g., as described in further detail above. In some embodiments, the report is transmitted via a computer network or a peer-to-peer connection. In some embodiments, an individual is administered a treatment based, at least in part, on the report. In some instances, all or a portion of the report may be displayed in a graphical user interface of an online or web-based healthcare portal.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. Articles of Manufacture or Kits

Provided herein are kits or articles of manufacture comprising one or more reagents for detecting a CD274 gene copy number alteration, such as a CD274 gene copy number gain, and a high tumor mutational burden in one or more samples. In some embodiments, the kits or articles of manufacture comprise one or more probes, baits, or oligonucleotides of the disclosure for detecting a CD274 gene copy number alteration, such as a CD274 gene copy number gain, and/or a high tumor mutational burden in one or more samples. In some embodiments, the kit is for use according to any method of detecting a CD274 gene copy number alteration, such as a CD274 gene copy number gain, and/or a high tumor mutational burden known in the art or described herein. In some embodiments, a kit provided herein further comprises instructions for detecting a CD274 gene copy number alteration, such as a CD274 gene copy number gain, and/or a high tumor mutational burden in one or more samples.

Further provided herein are kits or articles of manufacture comprising an anti-cancer therapy, such as an anti-cancer therapy described herein, e.g., an immunotherapy, such as an immune checkpoint inhibitor, and a package insert comprising instructions for using the anti-cancer therapy in a method of treating or delaying progression of cancer, e.g., by administration to an individual from whom sample(s) comprising a CD274 gene copy number alteration, such as a CD274 gene copy number gain, and/or a high tumor mutational burden, has been obtained. In some embodiments, the anti-cancer therapy is any of the anti-cancer therapies described herein (e.g., an immunotherapy, such as immune checkpoint inhibitor) for use in any of the methods for treating or delaying progression of cancer of the disclosure. In some embodiments, the anti-cancer therapy is an immune checkpoint inhibitor.

The kits or articles of manufacture of the disclosure may include, for example, a container and a label or package insert on or associated with the container. Suitable containers include, for example, bottles, vials, syringes, and the like. The container may be formed from a variety of materials such as glass or plastic. The container holds or contains a composition comprising one or more reagents for detecting a CD274 gene copy number alteration, such as a CD274 gene copy number gain, and/or a high tumor mutational burden (e.g., one or more oligonucleotides, primers, probes, or baits of the present disclosure) or one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure also includes information or instructions, for example in the form of a package insert, indicating that the one or more reagents and/or anti-cancer therapies are used for detecting a CD274 gene copy number alteration, such as a CD274 gene copy number gain, and/or a high tumor mutational burden, or for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. Nucleic Acids, Vectors, Host Cells and Recombinant Cells

Provided herein are nucleic acids and vectors comprising or encoding a bait, a probe, or an oligonucleotide described herein, or fragments thereof. In some embodiments, a vector provided herein is a nucleic acid molecule capable of transporting another nucleic acid to which it has been linked (e.g., baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising baits, probes, or oligonucleotides described herein, or fragments thereof) are also contemplated herein, including, e.g., replication defective retroviruses, adenoviruses and adeno-associated viruses.

In some embodiments, a nucleic acid or vector provided herein comprises a bait, a probe, or an oligonucleotide of the disclosure in a form suitable for expression thereof in a host cell. In some embodiments, the nucleic acid or vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., baits, probes, or oligonucleotides described herein, or fragments thereof). In some embodiments, a regulatory sequence directs tissue-specific expression of a nucleotide sequence (e.g., baits, probes, or oligonucleotides described herein, or fragments thereof). In some embodiments, a regulatory sequence directs inducible expression of a nucleotide sequence (e.g., baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises or encodes a bait, a probe, or an oligonucleotide of the disclosure in the sense or the anti-sense orientation. In some embodiments, a nucleic acid or vector (e.g., an expression vector) provided herein is introduced into host cells to thereby produce a polypeptide, or a fragment or mutant form thereof. In some embodiments, the design of a nucleic acid or vector provided herein depends on such factors as the choice of the host cell to be transformed, the level of expression desired, and the like. In some embodiments, expression vectors are designed for the expression of the baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises a nucleotide sequence that has been altered (e.g., codon optimized) so that the individual codons for each encoded amino acid are those preferentially utilized in the host cell.

Also provided herein are host cells, e.g., comprising baits, probes, nucleic acids, vectors, or oligonucleotides of the disclosure. In some embodiments, a host cell (e.g., a recombinant host cell or recombinant cell) comprises a vector described herein (e.g., an expression vector described herein). In some embodiments, a bait, probe, nucleic acid, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell. Baits, probes, nucleic acids, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

### VI. Exemplary Embodiments

The following exemplary embodiments are representative of some aspects of the invention:

Exemplary Embodiment 1: A method of identifying an individual having a cancer who may benefit from a treatment comprising an immunotherapy, the method comprising detecting in one or more samples from the individual a cluster of differentiation 274 (CD274) gene copy number gain and a high tumor mutational burden (TMB), wherein detection of the CD274 gene copy number gain and the high TMB in the one or more samples identifies the individual as one who may benefit from a treatment comprising an immunotherapy.

Exemplary Embodiment 2: A method of selecting a treatment for an individual having a cancer, the method comprising detecting in one or more samples from the individual a CD274 gene copy number gain and a high TMB, wherein detection of the CD274 gene copy number gain and the high TMB in the one or more samples identifies the individual as one who may benefit from a treatment comprising an immunotherapy.

Exemplary Embodiment 3: A method of identifying one or more treatment options for an individual having a cancer, the method comprising:
(a) detecting in one or more samples from the individual a CD274 gene copy number gain and a high TMB; and
(b) generating a report comprising one or more treatment options identified for the individual based, at least in part, on detection of the CD274 gene copy number gain and the high TMB in the one or more samples, wherein the one or more treatment options comprise an immunotherapy.

Exemplary Embodiment 4: A method of identifying one or more treatment options for an individual having a cancer, the method comprising:
(a) acquiring knowledge of a CD274 gene copy number gain and a high TMB in one or more samples from the individual; and
(b) generating a report comprising one or more treatment options identified for the individual based, at least in part, on said knowledge, wherein the one or more treatment options comprise an immunotherapy.

Exemplary Embodiment 5: The method of embodiment 3 or embodiment 4, wherein the report further indicates the presence or absence of the CD274 gene copy number gain and the high TMB in the one or more samples.

Exemplary Embodiment 6: A method of selecting a treatment for an individual having a cancer, comprising acquiring knowledge of a CD274 gene copy number gain and a high TMB in one or more samples from the individual, wherein responsive to the acquisition of said knowledge: (i) the individual is classified as a candidate to receive a treatment comprising an immunotherapy; and/or (ii) the individual is identified as likely to respond to a treatment that comprises an immunotherapy.

Exemplary Embodiment 7: A method of predicting survival of an individual having a cancer, comprising acquiring knowledge of a CD274 gene copy number gain and a high TMB in one or more samples from the individual, wherein responsive to the acquisition of said knowledge, the individual is predicted to have longer survival when treated with a treatment comprising an immunotherapy, as compared to survival of an individual whose cancer does not comprise a CD274 gene copy number gain and/or a high TMB.

Exemplary Embodiment 8: A method of predicting survival of an individual having a cancer treated with a treatment comprising an immunotherapy, the method comprising acquiring knowledge of a CD274 gene copy number gain and a high TMB in one or more samples from the individual, wherein responsive to the acquisition of said knowledge, the individual is predicted to have longer survival when treated with a treatment comprising an immunotherapy, as compared to an individual whose cancer does not exhibit a CD274 gene copy number gain and/or a high TMB.

Exemplary Embodiment 9: A method of treating or delaying progression of a cancer in an individual, comprising:
(a) acquiring knowledge of a CD274 gene copy number gain and a high TMB in one or more samples from an individual having a cancer; and
(b) responsive to said knowledge, administering to the individual an effective amount of a treatment that comprises an immunotherapy.

Exemplary Embodiment 10: A method of treating or delaying progression of a cancer in an individual, comprising administering to an individual having a cancer an effective amount of a treatment that comprises an immunotherapy, wherein the immunotherapy is administered responsive to acquiring knowledge of a CD274 gene copy number gain and a high TMB in one or more samples from the individual.

Exemplary Embodiment 11: A method of monitoring, evaluating or screening an individual having a cancer, comprising acquiring knowledge of a CD274 gene copy number gain and a high TMB in one or more samples from the individual, wherein responsive to the acquisition of said knowledge, the individual is predicted to have an improved response to a treatment comprising an immunotherapy and/or longer survival when treated with a treatment comprising an immunotherapy, as compared to an individual whose cancer does not comprise a CD274 gene copy number gain and/or a high TMB.

Exemplary Embodiment 12: A method for monitoring progression or recurrence of a cancer in an individual, the method comprising:
(a) detecting the presence or absence of a CD274 gene copy number gain and a high TMB in one or more samples obtained from the individual at a first time point;
(b) detecting the presence or absence of a CD274 gene copy number gain and a high TMB in one or more samples obtained from the individual at a second time point after the first time point; and (c) providing an assessment of cancer progression or cancer recurrence in the individual based, at least in part, on the presence or absence of the CD274 gene copy number gain and the high TMB in the one or more samples obtained from the individual at the first and/or second time points.

Exemplary Embodiment 13: The method of embodiment 12, wherein the presence of the CD274 gene copy number gain and the high TMB in the one or more samples obtained from the individual at the first and/or second time points identifies the individual as having decreased risk of cancer progression or cancer recurrence when treated with a treatment comprising an immunotherapy.

Exemplary Embodiment 14: The method of embodiment 12 or embodiment 13, further comprising selecting a treatment, administering a treatment, adjusting a treatment, adjusting a dose of a treatment, or applying a treatment to the individual based, at least in part, on detecting the presence of the CD274 gene copy number gain and the high TMB in the one or more samples obtained from the individual at the first and/or second time points, wherein the treatment comprises an immunotherapy.

Exemplary Embodiment 15: A method of identifying a candidate treatment for a cancer in an individual in need thereof, comprising:
performing DNA sequencing on one or more samples obtained from the individual to determine a sequencing mutation profile, wherein the sequencing mutation profile identifies the presence of a CD274 gene copy number gain and a high TMB in the one or more samples; and
selecting a treatment for the individual based, at least in part, on the sequencing mutation profile, wherein the treatment comprises an immunotherapy.

Exemplary Embodiment 16: The method of embodiment 15, wherein the presence of the CD274 gene copy number gain and the high TMB in the one or more samples identifies the individual as one who may benefit from a treatment comprising an immunotherapy.

Exemplary Embodiment 17: The method of embodiment 15 or embodiment 16, wherein the presence of the CD274 gene copy number gain and the high TMB in the one or more samples predicts the individual to have longer survival when treated with a treatment comprising an immunotherapy, as compared to survival of an individual whose cancer does not comprise a CD274 gene copy number gain and a high TMB.

Exemplary Embodiment 18: The method of any one of embodiments 15-17, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 19: The method of embodiment 18, wherein the sequencing comprises a massively parallel sequencing technique, and the massively parallel sequencing technique comprises next-generation sequencing (NGS).

Exemplary Embodiment 20: A method of treating or delaying progression of a cancer in an individual, comprising:
(a) detecting in one or more samples from an individual having a cancer a CD274 gene copy number gain and a high TMB; and
(b) administering to the individual an effective amount of a treatment that comprises an immunotherapy.

Exemplary Embodiment 21: The method of any one of embodiments 1-20, wherein the CD274 gene copy number gain in a sample from the individual comprises a CD274 gene copy number of any of at least +1, at least +2, at least +3, at least +4, at least +5, at least +6, at least +7, at least +8, or more, as compared to the ploidy of the sample from the individual.

Exemplary Embodiment 22: The method of embodiment 21, wherein the CD274 gene copy number gain in a sample from the individual comprises a CD274 gene copy number of any of at least +2, at least +3, at least +4, at least +5, at least +6, at least +7, at least +8, or more, as compared to the ploidy of the sample from the individual.

Exemplary Embodiment 23: The method of embodiment 21, wherein the CD274 gene copy number gain in a sample from the individual comprises a CD274 gene copy number of at least +2, as compared to the ploidy of the sample from the individual.

Exemplary Embodiment 24: The method of any one of embodiments 21-23, wherein the ploidy of the sample from the individual is monoploid, diploid, triploid, tetraploid.

Exemplary Embodiment 25: The method of any one of embodiments 1-20, wherein: the CD274 gene copy number gain in a sample from the individual comprises a CD274 gene copy number of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is monoploid; the CD274 gene copy number gain in a sample from the individual comprises a CD274 gene copy number of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is diploid; the CD274 gene copy number gain in a sample from the individual comprises a CD274 gene copy number of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is triploid; or the CD274 gene copy number gain in a sample from the individual comprises a CD274 gene copy number of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is tetraploid.

Exemplary Embodiment 26: The method of embodiment 25, wherein the CD274 gene copy number is any of at least four, at least five, at least six, or more.

Exemplary Embodiment 27: The method of embodiment 25, wherein the CD274 gene copy number is at least four.

Exemplary Embodiment 28: The method of any one of embodiments 4-11 and 21-27, wherein acquiring knowledge of a CD274 gene copy number gain and a high TMB comprises detecting the CD274 gene copy number gain and the high TMB in the one or more samples.

Exemplary Embodiment 29: The method of any one of embodiments 1-3, 5, 12-14, and 20-28, wherein the CD274 gene copy number gain is detected by fluorescence in situ hybridization (FISH), comprehensive genomic profiling (CGP), comparative genomic hybridization (CGH), sequencing, or any combination thereof.

Exemplary Embodiment 30: The method of embodiment 29, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 31: The method of embodiment 30, wherein the sequencing comprises a massively parallel sequencing technique, and the massively parallel sequencing technique comprises next-generation sequencing (NGS).

Exemplary Embodiment 32: The method of any one of embodiments 1-3, 5, 12-14, and 20-31, wherein detecting a CD274 gene copy number gain comprises:
(a) providing a plurality of nucleic acid molecules obtained from a sample from an individual having a cancer, wherein the plurality of nucleic acid molecules comprises nucleic acid molecules corresponding to a CD274 gene, or a portion thereof;
(b) optionally, ligating one or more adapters onto one or more nucleic acid molecules from the plurality of nucleic acid molecules;
(c) optionally, amplifying the one or more ligated nucleic acid molecules from the plurality of nucleic acid molecules;
(d) optionally, capturing amplified nucleic acid molecules from the amplified nucleic acid molecules;
(e) sequencing, by a sequencer, the captured nucleic acid molecules to obtain a plurality of sequence reads that represent the captured nucleic acid molecules, wherein one or more sequence reads of the plurality of sequence reads correspond to a CD274 gene, or a portion thereof;
(f) analyzing the plurality of sequence reads for the presence or absence of a CD274 gene copy number gain; and
(g) based on the analyzing step, detecting the presence or absence of a CD274 gene copy number gain in the sample.

Exemplary Embodiment 33: The method of embodiment 32, wherein the sequencer comprises a next-generation sequencer.

Exemplary Embodiment 34: The method of any one of embodiments 1-3, 5, 12-14, and 20-31, wherein detecting a CD274 gene copy number gain comprises:
(a) providing a sample from an individual having a cancer, wherein the sample comprises a plurality of nucleic acid molecules;
(b) preparing a nucleic acid sequencing library from the plurality of nucleic acid molecules in the sample;
(c) amplifying said library;
(d) selectively enriching for one or more nucleic acid molecules comprising nucleotide sequences corresponding to a CD274 gene or a portion thereof in said library to produce an enriched sample;
(e) sequencing the enriched sample, thereby producing a plurality of sequence reads;
(f) analyzing the plurality of sequence reads for the presence or absence of a CD274 gene copy number gain; and
(g) detecting, based on the analyzing step, the presence or absence of a CD274 gene copy number gain in the sample from the individual.

Exemplary Embodiment 35: The method of any one of embodiments 32-33, wherein the one or more adapters comprise amplification primers, flow cell adapter sequences, substrate adapter sequences, sample index sequences, or unique molecular identifier (UMI) sequences.

Exemplary Embodiment 36: The method of embodiment 34, wherein the selectively enriching comprises: (a) combining one or more bait molecules with the library, thereby hybridizing the one or more bait molecules to one or more nucleic acid molecules comprising nucleotide sequences corresponding to a CD274 gene or a portion thereof, and producing nucleic acid hybrids; and (b) isolating the nucleic acid hybrids to produce the enriched sample.

Exemplary Embodiment 37: The method of any one of embodiments 32-33, wherein the amplified nucleic acid molecules are captured by hybridization with one or more bait molecules.

Exemplary Embodiment 38: The method of any one of embodiments 32-37, wherein the amplifying comprises performing a polymerase chain reaction (PCR) amplification technique, a non-PCR amplification technique, or an isothermal amplification technique.

Exemplary Embodiment 39: The method of any one of embodiments 1-3, 5, 12-14, and 20-31, further comprising selectively enriching for one or more nucleic acid molecules in the sample comprising nucleotide sequences corresponding to a CD274 gene or a portion thereof; wherein the selectively enriching produces an enriched sample.

Exemplary Embodiment 40: The method of embodiment 39, wherein the selectively enriching comprises: (a) combining one or more bait molecules with the sample, thereby hybridizing the one or more bait molecules to one or more nucleic acid molecules in the sample comprising nucleotide sequences corresponding to a CD274 gene or a portion thereof and producing nucleic acid hybrids; and (b) isolating the nucleic acid hybrids to produce the enriched sample.

Exemplary Embodiment 41: The method of any one of embodiments 36-38 and 40, wherein the one or more bait molecules comprise a capture nucleic acid molecule configured to hybridize to a nucleotide sequence corresponding to a CD274 gene or a portion thereof.

Exemplary Embodiment 42: The method of embodiment 41, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Exemplary Embodiment 43: The method of any one of embodiments 36-38 and 40-42, wherein the one or more bait molecules are conjugated to an affinity reagent or to a detection reagent.

Exemplary Embodiment 44: The method of embodiment 43, wherein the affinity reagent is an antibody, an antibody fragment, or biotin, or wherein the detection reagent is a fluorescent marker.

Exemplary Embodiment 45: The method of any one of embodiments 41-44, wherein the capture nucleic acid molecule comprises a DNA, RNA, or mixed DNA/RNA molecule.

Exemplary Embodiment 46: The method of any one of embodiments 34 and 38-39, wherein the selectively enriching comprises amplifying the one or more nucleic acid molecules comprising nucleotide sequences corresponding to a CD274 gene or a portion thereof using a polymerase chain reaction (PCR) to produce an enriched sample.

Exemplary Embodiment 47: The method of any one of embodiments 39-46, further comprising sequencing the enriched sample.

Exemplary Embodiment 48: The method of any one of embodiments 32-47, wherein the plurality of nucleic acid molecules comprises a mixture of cancer nucleic acid molecules and non-cancer nucleic acid molecules.

Exemplary Embodiment 49: The method of embodiment 48, wherein the cancer nucleic acid molecules are derived from a tumor portion of a heterogeneous tissue biopsy sample, and the non-cancer nucleic acid molecules are derived from a normal portion of the heterogeneous tissue biopsy sample.

Exemplary Embodiment 50: The method of embodiment 48, wherein the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample.

Exemplary Embodiment 51: The method of any one of embodiments 32-50, wherein the analyzing step comprises:
(a) generating a copy number model based on the plurality of sequence reads; and
(b) determining a CD274 gene copy number based on the copy number model.

Exemplary Embodiment 52: The method of embodiment 51, wherein the copy number model is generated by:
(a) aligning the plurality of sequence reads against a reference genome;
(b) normalizing sequence coverage distribution of the aligned plurality of sequence reads against a control sample; and
(c) generating segmentation data for the normalized plurality of sequence reads.

Exemplary Embodiment 53: The method of any one of embodiments 32-50, wherein the analyzing step comprises: determining coverage ratio data, allele fraction data, and segmentation data for one or more gene loci within one or more subgenomic intervals of the plurality of sequence reads, wherein the one or more gene loci comprise CD274; identifying a plurality of segments based on the segmentation data; determining copy numbers for the plurality of segments based on the coverage ratio data, the allele fraction data, the segmentation data, and a copy number model; detecting the presence or absence of a CD274 copy number gain based on a copy number of a segment of the plurality of segments corresponding to CD274, wherein a CD274 copy number gain is detected when the copy number for the segment of the plurality of segments corresponding to CD274 is greater than ploidy of the sample.

Exemplary Embodiment 54: The method of embodiment 53, wherein the coverage ratio data is determined by aligning a plurality of sequence reads from the sample and from a control sample to a reference genome, and determining a number of sequence reads that overlap each of the one or more gene loci within the one or more subgenomic intervals in the sample and in the control sample.

Exemplary Embodiment 55: The method of embodiment 52 or 54, wherein the control sample is a paired normal sample, a process-matched control sample, or a panel of normal control sample.

Exemplary Embodiment 56: The method of any one of embodiments 51-55, wherein the copy number model: (a) predicts a copy number for CD274 based on coverage ratio data and allele fraction data; (b) predicts a sample purity and ploidy for the sample; (c) outputs segmentation data; and any combination thereof.

Exemplary Embodiment 57: The method of any one of embodiments 52-56, wherein the segmentation data is generated by aligning a plurality of sequence reads from the sample to a reference genome, and processing aligned sequence read data, coverage ratio data, and allele fraction data to determine a number of segments required to account for the aligned sequence read data, wherein each segment has a same copy number.

Exemplary Embodiment 58: The method of any one of embodiments 1-3, 5, 12-14, and 20-29, wherein the CD274 gene copy number gain is detected by fluorescence in situ hybridization (FISH) based on a ratio of fluorescence signal from a FISH probe that binds to CD274 and from a control FISH probe.

Exemplary Embodiment 59: The method of embodiment 58, wherein the control FISH probe is a centromere enumeration probe for chromosome 9 (CEP9).

Exemplary Embodiment 60: The method of embodiment 58 or embodiment 59, wherein the CD274 gene copy number gain comprises a ratio of fluorescence signal from a FISH probe that binds to CD274 to fluorescence signal from the control FISH probe of any of at least 2, at least 3, at least 4, at least 5, at least 6 or more.

Exemplary Embodiment 61: The method of any one of embodiments 1-3, 5, 12-14, and 20-60, wherein the high TMB is detected in a sample from the individual by sequencing.

Exemplary Embodiment 62: The method of embodiment 61, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 63: The method of embodiment 62, wherein the sequencing comprises a massively parallel sequencing technique, and the massively parallel sequencing technique comprises next-generation sequencing (NGS).

Exemplary Embodiment 64: The method of any one of embodiments 1-63, wherein TMB is assessed based on about 0.79 megabases (Mb) of sequenced DNA.

Exemplary Embodiment 65: The method of any one of embodiments 1-63, wherein TMB is assessed based on about 0.80 Mb of sequenced DNA.

Exemplary Embodiment 66: The method of any one of embodiments 1-63, wherein TMB is assessed based on between about 0.83 Mb and about 1.14 Mb of sequenced DNA.

Exemplary Embodiment 67: The method of any one of embodiments 1-63, wherein TMB is assessed based on about 1.1 Mb of sequenced DNA.

Exemplary Embodiment 68: The method of any one of embodiments 1-63, wherein TMB is assessed based on up to about 1.24 Mb of sequenced DNA.

Exemplary Embodiment 69: The method of any one of embodiments 1-63, wherein TMB is assessed based on up to about 1.1 Mb of sequenced DNA.

Exemplary Embodiment 70: The method of any one of embodiments 1-69, wherein the high TMB comprises a TMB of at least about 5 mutations/Megabase (mut/Mb) or at least about 10 mut/Mb.

Exemplary Embodiment 71: The method of embodiment 70, wherein the cancer comprises a TMB of any of at least about 5 mut/Mb, at least about 10 mut/Mb, at least about 20 mut/Mb, at least about 30 mut/Mb, at least about 40 mut/Mb, at least about 50 mut/Mb, at least about 60 mut/Mb, at least about 70 mut/Mb, at least about 80 mut/Mb, at least about 90 mut/Mb, at least about 100 mut/Mb, at least about 110 mut/Mb, at least about 120 mut/Mb, at least about 130 mut/Mb, at least about 140 mut/Mb, at least about 150 mut/Mb, or more.

Exemplary Embodiment 72: The method of any one of embodiments 1-2 and 6-71, further comprising generating a report, wherein the report: (a) indicates the presence of the CD274 gene copy number gain and high TMB in the one or more samples from the individual; and/or (b) indicates a treatment or one or more treatment options identified or selected for the individual based, at least in part, on the presence of the CD274 gene copy number gain and high TMB in the one or more samples from the individual, wherein the treatment or the one or more treatment options comprise an immunotherapy.

Exemplary Embodiment 73: The method of any one of embodiments 1-71, further comprising generating a molecular profile for the individual, based, at least in part, on detecting or acquiring knowledge of the CD274 gene copy number gain and/or high TMB.

Exemplary Embodiment 74: The method of embodiment 73, wherein the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 75: The method of embodiment 73 or embodiment 74, wherein the molecular profile for the individual further comprises results from a nucleic acid sequencing-based test.

Exemplary Embodiment 76: The method of any one of embodiments 73-75, further comprising selecting a treatment, administering a treatment, or applying a treatment to the individual based on the generated molecular profile, wherein the treatment comprises an immunotherapy.

Exemplary Embodiment 77: The method of any one of embodiments 73-76, further comprising generating a report, wherein the report comprises the molecular profile for the individual.

Exemplary Embodiment 78: The method of embodiment 77, wherein the report further comprises information on a treatment or one or more treatment options identified or selected for the individual based, at least in part, on the molecular profile for the individual, wherein the treatment or one or more treatment options comprise an immunotherapy.

Exemplary Embodiment 79: The method of any one of embodiments 3-5 and 21-78, further comprising providing the report to the individual, a caregiver, a healthcare provider, a physician, an oncologist, an electronic medical record system, a hospital, a clinic, a third-party payer, an insurance company, or a government office.

Exemplary Embodiment 80: The method of any one of embodiments 1-79, further comprising assessing microsatellite instability status of the cancer in a sample from the individual.

Exemplary Embodiment 81: The method of any one of embodiments 3-5 and 21-80, wherein the report further indicates the microsatellite instability status of the cancer.

Exemplary Embodiment 82: The method of any one of embodiments 73-80, wherein the molecular profile further indicates the microsatellite instability status of the cancer.

Exemplary Embodiment 83: The method of any one of embodiments 80-82, wherein microsatellite instability is assessed by sequencing, a polymerase chain reaction (PCR) amplification technique, a non-PCR amplification technique, an isothermal amplification technique, a capillary electrophoresis method, immunohistochemistry, and any combination thereof.

Exemplary Embodiment 84: The method of embodiment 83, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 85: The method of embodiment 84, wherein the sequencing comprises a massively parallel sequencing technique, and the massively parallel sequencing technique comprises next-generation sequencing (NGS).

Exemplary Embodiment 86: The method of any one of embodiments 1-85, wherein the cancer has high microsatellite instability (MSI-high).

Exemplary Embodiment 87: The method of any one of embodiments 1-85, wherein the cancer is microsatellite stable.

Exemplary Embodiment 88: The method of any one of embodiments 1-87, further comprising assessing expression of PD-L1 protein in a sample from the individual.

Exemplary Embodiment 89: The method of any one of embodiments 3-5 and 21-88, wherein the report further indicates the PD-L1 protein expression status of the cancer.

Exemplary Embodiment 90: The method of any one of embodiments 73-88, wherein the molecular profile further indicates the PD-L1 protein expression status of the cancer.

Exemplary Embodiment 91: The method of any one of embodiments 88-90, wherein PD-L1 protein expression is determined using an immunohistochemistry assay.

Exemplary Embodiment 92: The method of embodiment 91, wherein the immunohistochemistry assay is a DAKO PD-L1 22C3 assay.

Exemplary Embodiment 93: The method of embodiment 91 or embodiment 92, wherein PD-L1 expression is assessed based on a tumor proportion score (TPS).

Exemplary Embodiment 94: The method of embodiment 93, wherein the cancer is PD-L1 positive.

Exemplary Embodiment 95: The method of embodiment 94, wherein the cancer has a TPS of at least about 1%.

Exemplary Embodiment 96: The method of embodiment 94, wherein the cancer has a TPS of between about 1% and about 49%.

Exemplary Embodiment 97: The method of embodiment 94, wherein the cancer has a TPS of at least about 50%.

Exemplary Embodiment 98: The method of embodiment 94, wherein the cancer has a TPS of at least about 1%, at least about 25%, at least about 50%, or at least about 75%.

Exemplary Embodiment 99: The method of embodiment 94, wherein the cancer has a TPS of at least about 1%, at least about 5%, at least about 10%, at least about 20%, at least about 30%, at least about 40%, at least about 50%, at least about 60%, at least about 70%, at least about 80%, at least about 90%, at least about 95%, at least about 99%, or 100%.

Exemplary Embodiment 100: The method of embodiment 93, wherein the cancer is PD-L1 negative.

Exemplary Embodiment 101: The method of embodiment 100, wherein the cancer has a TPS of less than 1%.

Exemplary Embodiment 102: The method of embodiment 91 or embodiment 92, wherein PD-L1 expression is assessed based on a combined positive score (CPS).

Exemplary Embodiment 103: The method of embodiment 102, wherein the cancer is PD-L1 positive.

Exemplary Embodiment 104: The method of embodiment 103, wherein the cancer has a CPS of at least about 1 or at least about 10.

Exemplary Embodiment 105: The method of embodiment 102, wherein the cancer is PD-L1 negative.

Exemplary Embodiment 106: The method of embodiment 105, wherein the cancer has a CPS of less than 1.

Exemplary Embodiment 107: The method of embodiment 91, wherein the immunohistochemistry assay is a VENTANA SP 142 assay.

Exemplary Embodiment 108: The method of embodiment 107, wherein PD-L1 expression is assessed based on the proportion of tumor area occupied by PD-L1-expressing tumor-infiltrating immune cells of any intensity (IC), or the percentage of PD-L1-expressing tumor cells of any intensity (TC).

Exemplary Embodiment 109: The method of embodiment 108, wherein the cancer is PD-L1 positive.

Exemplary Embodiment 110: The method of embodiment 109, wherein the cancer has a TC or IC of at least about 1%.

Exemplary Embodiment 111: The method of embodiment 108, wherein the cancer is PD-L1 negative.

Exemplary Embodiment 112: The method of embodiment 111, wherein the cancer has a TC or IC of less than 1%.

Exemplary Embodiment 113: The method of any one of embodiments 1-112, further comprising acquiring knowledge of or detecting in a sample from the individual a base substitution, a short insertion/deletion (indel), a copy number alteration, or a genomic rearrangement in one or more genes; optionally wherein the one or more genes comprise CD274, EGFR and/or ALK.

Exemplary Embodiment 114: The method of any one of embodiments 1-113, wherein the individual is a human.

Exemplary Embodiment 115: The method of any one of embodiments 1-114, wherein the cancer is a carcinoma, a sarcoma, a lymphoma, a leukemia, a myeloma, a germ cell cancer, or a blastoma.

Exemplary Embodiment 116: The method of any one of embodiments 1-115, wherein the cancer is a solid tumor.

Exemplary Embodiment 117: The method of any one of embodiments 1-115, wherein the cancer is a hematologic malignancy.

Exemplary Embodiment 118: The method of any one of embodiments 1-117, wherein the cancer is a B cell cancer, multiple myeloma, melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Exemplary Embodiment 119: The method of any one of embodiments 1-117, wherein the cancer is a non-small cell lung cancer.

Exemplary Embodiment 120: The method of embodiment 119, wherein the cancer is a non-squamous non-small cell lung cancer.

Exemplary Embodiment 121: The method of embodiment 120, wherein the CD274 gene copy number gain in a sample from the individual comprises a CD274 gene copy number of at least +2, as compared to the ploidy of the sample from the individual, optionally wherein the ploidy of the sample is diploid; and wherein the cancer comprises a TMB of at least about 10 mut/Mb.

Exemplary Embodiment 122: The method of embodiment 120, wherein the CD274 gene copy number gain in a sample from the individual comprises a CD274 gene copy number of at least 4, wherein the ploidy of the sample from the individual is diploid; and wherein the cancer comprises a TMB of at least about 10 mut/Mb.

Exemplary Embodiment 123: The method of embodiment 121 or embodiment 122, wherein administering a treatment comprising an immunotherapy to the individual results in survival of the individual for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 25 months, or more, measured from the start of treatment with the immunotherapy.

Exemplary Embodiment 124: The method of any one of embodiments 121-123, wherein administering a treatment comprising an immunotherapy to a plurality of individuals results in a median overall survival of the individuals in the plurality of at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 25 months, or more, measured from the start of treatment with the immunotherapy.

Exemplary Embodiment 125: The method of any one of embodiments 119-124, wherein the cancer is a Stage I, Stage II, Stage III, Stage IV, or unknown stage cancer.

Exemplary Embodiment 126: The method of any one of embodiments 119-125, wherein the individual has a history smoking, or does not have a history of smoking.

Exemplary Embodiment 127: The method of any one of embodiments 119-126, wherein the cancer is metastatic.

Exemplary Embodiment 128: The method of any one of embodiments 119-127, wherein the individual has an Eastern Cooperative Oncology Group status of any of 0, 1, 2, 3, or more.

Exemplary Embodiment 129: The method of any one of embodiments 1-128, wherein the cancer does not comprise any oncogenic alterations in an EGFR and/or an ALK gene.

Exemplary Embodiment 130: The method of any one of embodiments 1-129, wherein the cancer is wild type for oncogenic alterations in an EGFR and/or an ALK gene.

Exemplary Embodiment 131: The method of embodiment 129 or embodiment 130, wherein the oncogenic alterations comprise base substitutions, insertions/deletions, copy number alterations, or rearrangements.

Exemplary Embodiment 132: The method of any one of embodiments 1-131, wherein the individual was previously treated with a first-line treatment for the cancer with an anti-VEGF chemotherapy combination treatment, an EGFR tyrosine kinase inhibitor, a platinum-based chemotherapy, or a single agent chemotherapy.

Exemplary Embodiment 133: The method of any one of embodiments 1-132, wherein the immunotherapy is an immune checkpoint inhibitor, a cancer vaccine, a cell-based therapy, a T cell receptor (TCR)-based therapy, an adjuvant immunotherapy, a cytokine immunotherapy, or an oncolytic virus therapy

Exemplary Embodiment 134: The method of any one of embodiments 1-133, wherein the immunotherapy is a monotherapy.

Exemplary Embodiment 135: The method of embodiment 133 or embodiment 134, wherein the immune checkpoint inhibitor is a first-line immune checkpoint inhibitor.

Exemplary Embodiment 136: The method of any one of embodiments 133-135, wherein the immune checkpoint inhibitor is a second-line immune checkpoint inhibitor.

Exemplary Embodiment 137: The method of any one of embodiments 133-136, wherein the immune checkpoint inhibitor is a PD-1- or a PD-L1-targeted agent.

Exemplary Embodiment 138: The method of embodiment 137, wherein the immune checkpoint inhibitor is a PD-1 inhibitor.

Exemplary Embodiment 139: The method of embodiment 138, wherein the immune checkpoint inhibitor comprises one or more of nivolumab, pembrolizumab, cemiplimab, or dostarlimab.

Exemplary Embodiment 140: The method of embodiment 137, wherein the immune checkpoint inhibitor is a PD-L1-inhibitor.

Exemplary Embodiment 141: The method of embodiment 140, wherein the immune checkpoint inhibitor comprises one or more of atezolizumab, avelumab, or durvalumab.

Exemplary Embodiment 142: The method of any one of embodiments 133-136, wherein the immune checkpoint inhibitor is a CTLA-4 inhibitor.

Exemplary Embodiment 143: The method of embodiment 142, wherein the CTLA-4 inhibitor comprises ipilimumab.

Exemplary Embodiment 144: The method of any one of embodiments 1-143, wherein the cancer in the individual has not been previously treated with an immune checkpoint inhibitor.

Exemplary Embodiment 145: The method of any one of embodiments 1-144, wherein the treatment or the one or more treatment options comprise an additional anti-cancer therapy.

Exemplary Embodiment 146: The method of embodiment 145, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Exemplary Embodiment 147: The method of embodiment 146, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 148: The method of embodiment 146, wherein the nucleic acid comprises a double-stranded RNA (dsRNA), a small interfering RNA (siRNA), or a small hairpin RNA (shRNA).

Exemplary Embodiment 149: The method of any one of embodiments 1-148, further comprising obtaining the one or more samples from the individual.

Exemplary Embodiment 150: The method of any one of embodiments 1-149, wherein the one or more samples are obtained or derived from the cancer.

Exemplary Embodiment 151: The method of any one of embodiments 1-150, wherein one or more samples from the individual comprise a tissue biopsy sample, a liquid biopsy sample, or a normal control.

Exemplary Embodiment 152: The method of any one of embodiments 1-150, wherein one or more samples from the individual are from a tumor biopsy, tumor specimen, or circulating tumor cell.

Exemplary Embodiment 153: The method of any one of embodiments 1-150, wherein one or more samples from the individual are a liquid biopsy sample comprising blood, plasma, cerebrospinal fluid, sputum, stool, urine, or saliva.

Exemplary Embodiment 154: The method of any one of embodiments 1-153, wherein one or more samples from the individual comprise cells and/or nucleic acids from the cancer.

Exemplary Embodiment 155: The method of embodiment 154, wherein one or more samples from the individual comprise at least 20% tumor cell nuclear area.

Exemplary Embodiment 156: The method of embodiment 154, wherein one or more samples from the individual comprise mRNA, DNA, circulating tumor DNA (ctDNA), cell-free DNA, or cell-free RNA from the cancer.

Exemplary Embodiment 157: The method of any one of embodiments 1-150, wherein one or more samples from the individual are a liquid biopsy sample comprising circulating tumor cells (CTCs).

Exemplary Embodiment 158: The method of any one of embodiments 1-150, wherein one or more samples from the individual are a liquid biopsy sample comprising cell-free DNA (cfDNA), circulating tumor DNA (ctDNA), or any combination thereof.

Exemplary Embodiment 159: A system for identifying an individual having a cancer who may benefit from a treatment comprising an immunotherapy, the system comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from one or more samples obtained from an individual having a cancer;
(b) analyze the plurality of sequence reads for the presence of a CD274 gene copy number gain and a high TMB; and
(c) detect, based on the analyzing, the presence of the CD274 gene copy number gain and high TMB in the one or more samples;
wherein detecting the CD274 gene copy number gain and high TMB identifies the individual as one who may benefit from a treatment comprising an immunotherapy.

Exemplary Embodiment 160: A non-transitory computer readable storage medium comprising one or more programs executable by one or more computer processors for performing a method for identifying an individual having a cancer who may benefit from a treatment comprising an immunotherapy, the method comprising:
(a) obtaining, using the one or more processors, a plurality of sequence reads of one or more nucleic acid molecules, wherein the one or more nucleic acid molecules are derived from one or more samples obtained from an individual having a cancer;
(b) analyzing, using the one or more processors, the plurality of sequence reads for the presence of a CD274 gene copy number gain and a high TMB; and
(c) detecting, using the one or more processors and based on the analyzing, the CD274 gene copy number gain and high TMB in the one or more samples;
wherein detecting the CD274 gene copy number gain and high TMB identifies the individual as one who may benefit from a treatment comprising an immunotherapy.

Exemplary Embodiment 161: The system of embodiment 159, or the non-transitory computer readable storage medium of embodiment 160, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next-generation sequencing (NGS).

Exemplary Embodiment 162: The system of embodiment 159 or embodiment 161, or the non-transitory computer readable storage medium of embodiment 160 or embodiment 161, wherein the analyzing step comprises:
(a) generating a copy number model based on the plurality of sequence reads; and
(b) determining a CD274 gene copy number based on the copy number model.

Exemplary Embodiment 163: The system or the non-transitory computer readable storage medium of embodiment 162, wherein the copy number model is generated by:
(a) aligning the plurality of sequence reads against a reference genome;
(b) normalizing sequence coverage distribution of the aligned plurality of sequence reads against a control sample; and
(c) generating segmentation data for the normalized plurality of sequence reads.

Exemplary Embodiment 164: The system of embodiment 159 or embodiment 161, or the non-transitory computer readable storage medium of embodiment 160 or embodiment 161, wherein the analyzing step comprises: determining coverage ratio data, allele fraction data, and segmentation data for one or more gene loci within one or more subgenomic intervals of the plurality of sequence reads, wherein the one or more gene loci comprise CD274; identifying a plurality of segments based on the segmentation data; determining copy numbers for the plurality of segments based on the coverage ratio data, the allele fraction data, the segmentation data, and a copy number model; detecting the presence or absence of a CD274 copy number gain based on a copy number of a segment of the plurality of segments corresponding to CD274, wherein a CD274 copy number gain is detected when the copy number for the segment of the plurality of segments corresponding to CD274 is greater than ploidy of the sample.

Exemplary Embodiment 165: The system or the non-transitory computer readable storage medium of embodiment 164, wherein the coverage ratio data is determined by aligning a plurality of sequence reads from the sample and from a control sample to a reference genome, and determining a number of sequence reads that overlap each of the one or more gene loci within the one or more subgenomic intervals in the sample and in the control sample.

Exemplary Embodiment 166: The system or the non-transitory computer readable storage medium of embodiment 163 or 165, wherein the control sample is a paired normal sample, a process-matched control sample, or a panel of normal control sample.

Exemplary Embodiment 167: The system or the non-transitory computer readable storage medium of any one of embodiments 162-166, wherein the copy number model: (a) predicts a copy number for CD274 based on coverage ratio data and allele fraction data; (b) predicts a sample purity and ploidy for the sample; (c) outputs segmentation data; and any combination thereof.

Exemplary Embodiment 168: The system or the non-transitory computer readable storage medium of any one of embodiments 163-167, wherein the segmentation data is generated by aligning a plurality of sequence reads from the sample to a reference genome, and processing aligned sequence read data, coverage ratio data, and allele fraction data to determine a number of segments required to account for the aligned sequence read data, wherein each segment has a same copy number.

Exemplary Embodiment 169: The system of any one of embodiments 159 and 161-168, or the non-transitory computer readable storage medium of any one of embodiments 160-168, wherein the CD274 gene copy number gain comprises a CD274 gene copy number of any of at least +1, at least +2, at least +3, at least +4, at least +5, at least +6, at least +7, at least +8, or more, as compared to the ploidy of a sample from the individual.

Exemplary Embodiment 170: The system or the non-transitory computer readable storage medium of embodiment 169, wherein the CD274 gene copy number gain comprises a CD274 gene copy number of any of at least +2, at least +3, at least +4, at least +5, at least +6, at least +7, at least +8, or more, as compared to the ploidy of a sample from the individual.

Exemplary Embodiment 171: The system or the non-transitory computer readable storage medium of embodiment 169, wherein the CD274 gene copy number gain comprises a CD274 gene copy number of at least +2, as compared to the ploidy of a sample from the individual.

Exemplary Embodiment 172: The system or the non-transitory computer readable storage medium of any one of embodiments 169-171, wherein the ploidy of the sample from the individual is monoploid, diploid, triploid, or tetraploid.

Exemplary Embodiment 173: The system of any one of embodiments 159 and 161-168, or the non-transitory computer readable storage medium of any one of embodiments 160-168, wherein: the CD274 gene copy number gain in a sample from the individual comprises a CD274 gene copy number of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is monoploid; the CD274 gene copy number gain in a sample from the individual comprises a CD274 gene copy number of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is diploid; the CD274 gene copy number gain in a sample from the individual comprises a CD274 gene copy number of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is triploid; or the CD274 gene copy number gain in a sample from the individual comprises a CD274 gene copy number of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is tetraploid.

Exemplary Embodiment 174: The system or the non-transitory computer readable storage medium of embodiment 173, wherein the CD274 gene copy number is any of at least four, at least five, at least six, or more.

Exemplary Embodiment 175: The system or the non-transitory computer readable storage medium of embodiment 173, wherein the CD274 copy number is at least four.

Exemplary Embodiment 176: The system of any one of embodiments 159 and 161-175, or the non-transitory computer readable storage medium of any one of embodiments 160-175, wherein TMB is assessed based on about 0.79 megabases (Mb) of sequenced DNA.

Exemplary Embodiment 177: The system of any one of embodiments 159 and 161-175, or the non-transitory computer readable storage medium of any one of embodiments 160-175, wherein TMB is assessed based on about 0.80 Mb of sequenced DNA.

Exemplary Embodiment 178: The system of any one of embodiments 159 and 161-175, or the non-transitory computer readable storage medium of any one of embodiments 160-175, wherein TMB is assessed based on between about 0.83 Mb and about 1.14 Mb of sequenced DNA.

Exemplary Embodiment 179: The system of any one of embodiments 159 and 161-175, or the non-transitory computer readable storage medium of any one of embodiments 160-175, wherein TMB is assessed based on about 1.1 Mb of sequenced DNA.

Exemplary Embodiment 180: The system of any one of embodiments 159 and 161-175, or the non-transitory computer readable storage medium of any one of embodiments 160-175, wherein TMB is assessed based on up to about 1.24 Mb of sequenced DNA.

Exemplary Embodiment 181: The system of any one of embodiments 159 and 161-175, or the non-transitory computer readable storage medium of any one of embodiments 160-175, wherein TMB is assessed based on up to about 1.1 Mb of sequenced DNA.

Exemplary Embodiment 182: The system of any one of embodiments 159 and 161-181, or the non-transitory computer readable storage medium of any one of embodiments 160-181, wherein the high TMB comprises a TMB of at least about 5 mutations/Megabase (mut/Mb), or at least about 10 mut/Mb.

Exemplary Embodiment 183: The system or the non-transitory computer readable storage medium of embodiment 182, wherein the cancer comprises a TMB of any of at least about 5 mut/Mb, at least about 10 mut/Mb, at least about 20 mut/Mb, at least about 30 mut/Mb, at least about 40 mut/Mb, at least about 50 mut/Mb, at least about 60 mut/Mb, at least about 70 mut/Mb, at least about 80 mut/Mb, at least about 90 mut/Mb, at least about 100 mut/Mb, at least about 110 mut/Mb, at least about 120 mut/Mb, at least about 130 mut/Mb, at least about 140 mut/Mb, at least about 150 mut/Mb, or more.

Exemplary Embodiment 184: The system of any one of embodiments 159 and 161-183, or the non-transitory computer readable storage medium of any one of embodiments 160-183, wherein the individual is administered a treatment based, at least in part, on detecting the CD274 gene copy number gain and high TMB in the one or more samples.

Exemplary Embodiment 185: The system of any one of embodiments 159 and 161-184, wherein the one or more program instructions when executed by the one or more processors are further configured to generate, based at least in part on the detecting, a molecular profile for the individual.

Exemplary Embodiment 186: The non-transitory computer readable storage medium of any one of embodiments 160-184, wherein the method further comprises generating, based at least in part on the detecting, a molecular profile for the individual.

Exemplary Embodiment 187: The system of embodiment 185, or the non-transitory computer readable storage medium of embodiment 186, wherein the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 188: The system of embodiment 185 or embodiment 187, or the non-transitory computer readable storage medium of embodiment 186 or embodiment 187, wherein the molecular profile further comprises results from a nucleic acid sequencing-based test.

Exemplary Embodiment 189: The system of any one of embodiments 159, 161-185, and 187-188, wherein the one or more program instructions when executed by the one or more processors are further configured to generate a report, wherein the report indicates the presence of the CD274 gene copy number gain and high TMB in the one or more samples.

Exemplary Embodiment 190: The system of any one of embodiments 185 and 187-188, wherein the one or more program instructions when executed by the one or more processors are further configured to generate a report, wherein the report comprises the molecular profile.

Exemplary Embodiment 191: The non-transitory computer readable storage medium of any one of embodiments 160-184 and 186-188, wherein the method further comprises generating a report, wherein the report indicates the presence of the CD274 gene copy number gain and high TMB in the one or more samples.

Exemplary Embodiment 192: The non-transitory computer readable storage medium of any one of embodiments 186-188, wherein the method further comprises generating a report, wherein the report comprises the molecular profile.

Exemplary Embodiment 193: The system of embodiment 189 or embodiment 190, wherein the one or more program instructions when executed by the one or more processors are further configured to transmit the report to the individual, a caregiver, a healthcare provider, a physician, an oncologist, an electronic medical record system, a hospital, a clinic, a third-party payer, an insurance company, or a government office.

Exemplary Embodiment 194: The non-transitory computer readable storage medium of embodiment 191 or embodiment 192, wherein the method further comprises transmitting the report to the individual, a caregiver, a healthcare provider, a physician, an oncologist, an electronic medical record system, a hospital, a clinic, a third-party payer, an insurance company, or a government office.

Exemplary Embodiment 195: The system of embodiment 193, or the non-transitory computer readable storage medium of embodiment 194, wherein the report is transmitted via a computer network or a peer-to-peer connection.

Exemplary Embodiment 196: The system of any one of embodiments 189-190, 193 and 195, or the non-transitory computer readable storage medium of any one of embodiments 191-192 and 194-195, wherein the individual is administered a treatment based, at least in part, on the report.

Exemplary Embodiment 197: The system of any one of embodiments 185 and 187-188, or the non-transitory computer readable storage medium of any one of embodiments 186-188, wherein the individual is administered a treatment based, at least in part, on the molecular profile for the individual.

Exemplary Embodiment 198: The system or the non-transitory computer readable storage medium of any one of embodiments 184 and 196-197, wherein the treatment comprises an immunotherapy.

Exemplary Embodiment 199: The system of any one of embodiments 159, 161-185, 187-190, 193, and 195-198, or the non-transitory computer readable storage medium of any one of embodiments 160-184, 186-188, 191-192, and 194-198, wherein the immunotherapy is an immune checkpoint inhibitor, a cancer vaccine, a cell-based therapy, a T cell receptor (TCR)-based therapy, an adjuvant immunotherapy, a cytokine immunotherapy, or an oncolytic virus therapy.

Exemplary Embodiment 200: The system or the non-transitory computer readable storage medium of embodiment 198 or embodiment 199, wherein the immunotherapy is a monotherapy.

Exemplary Embodiment 201: The system or the non-transitory computer readable storage medium of embodiment 199 or embodiment 200, wherein the immune checkpoint inhibitor is a first-line immune checkpoint inhibitor.

Exemplary Embodiment 202: The system or the non-transitory computer readable storage medium of any one of embodiments 199-201, wherein the immune checkpoint inhibitor is a second-line immune checkpoint inhibitor.

Exemplary Embodiment 203: The system or the non-transitory computer readable storage medium of any one of embodiments 199-202, wherein the immune checkpoint inhibitor is a PD-1- or a PD-L1-targeted agent.

Exemplary Embodiment 204: The system or the non-transitory computer readable storage medium of embodiment 203, wherein the immune checkpoint inhibitor is a PD-1 inhibitor, a PD-L1-inhibitor, or a CTLA-4 inhibitor.

Exemplary Embodiment 205: The system or the non-transitory computer readable storage medium of embodiment 204, wherein the immune checkpoint inhibitor comprises one or more of nivolumab, pembrolizumab, cemiplimab, dostarlimab, atezolizumab, avelumab, durvalumab, or ipilimumab.

Exemplary Embodiment 206: The system of any one of embodiments 159, 161-185, 187-190, 193, and 195-205, or the non-transitory computer readable storage medium of any one of embodiments 160-184, 186-188, 191-192, and 194-205, wherein the individual is a human.

Exemplary Embodiment 207: The system of any one of embodiments 159, 161-185, 187-190, 193, and 195-206, or the non-transitory computer readable storage medium of any one of embodiments 160-184, 186-188, 191-192, and 194-206, wherein the cancer is a carcinoma, a sarcoma, a lymphoma, a leukemia, a myeloma, a germ cell cancer, or a blastoma.

Exemplary Embodiment 208: The system of any one of embodiments 159, 161-185, 187-190, 193, and 195-207, or the non-transitory computer readable storage medium of any one of embodiments 160-184, 186-188, 191-192, and 194-207, wherein the cancer is a solid tumor or a hematologic malignancy.

Exemplary Embodiment 209: The system of any one of embodiments 159, 161-185, 187-190, 193, and 195-208, or the non-transitory computer readable storage medium of any one of embodiments 160-184, 186-188, 191-192, and 194-208, wherein the cancer is a B cell cancer, multiple myeloma, melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Exemplary Embodiment 210: The system of any one of embodiments 159, 161-185, 187-190, 193, and 195-209, or the non-transitory computer readable storage medium of any one of embodiments 160-184, 186-188, 191-192, and 194-209, wherein the cancer is a non-small cell lung cancer.

Exemplary Embodiment 211: The system or the non-transitory computer readable storage medium of embodiment 210, wherein the cancer is a non-squamous non-small cell lung cancer.

Exemplary Embodiment 212: The system or the non-transitory computer readable storage medium of embodiment 211, wherein the CD274 gene copy number gain comprises a CD274 gene copy number of at least +2, as compared to the ploidy of a sample from the individual; and wherein the cancer comprises a TMB of at least about 10 mut/Mb.

Exemplary Embodiment 213: The system or the non-transitory computer readable storage medium of embodiment 211, wherein the CD274 gene copy number gain comprises a CD274 gene copy number of at least 4, wherein the ploidy of a sample from the individual is diploid; and wherein the cancer comprises a TMB of at least about 10 mut/Mb.

Exemplary Embodiment 214: The system of any one of embodiments 159, 161-185, 187-190, 193, and 195-213, or the non-transitory computer readable storage medium of any one of embodiments 160-184, 186-188, 191-192, and 194-213, wherein the cancer does not comprise any oncogenic alterations in an EGFR and/or an ALK gene.

Exemplary Embodiment 215: The system of any one of embodiments 159, 161-185, 187-190, 193, and 195-214, or the non-transitory computer readable storage medium of any one of embodiments 160-184, 186-188, 191-192, and 194-214, wherein the cancer is wild type for oncogenic alterations in an EGFR and/or an ALK gene.

Exemplary Embodiment 216: The system or the non-transitory computer readable storage medium of embodiment 214 or embodiment 215, wherein the oncogenic alterations comprise base substitutions, insertions/deletions, copy number alterations, or rearrangements

Exemplary Embodiment 217: An immunotherapy for use in a method of treating or delaying progression of cancer in an individual, wherein the method comprises administering the immunotherapy to an individual having a cancer, wherein a CD274 gene copy number gain and a high TMB are detected in one or more samples obtained from the individual.

Exemplary Embodiment 218: An immunotherapy for use in the manufacture of a medicament for treating or delaying progression of cancer in an individual, wherein the medicament is to be administered to an individual having a cancer, wherein a CD274 gene copy number gain and a high TMB are detected in one or more samples obtained from the individual.

Exemplary Embodiment 219: A method of identifying an individual having a cancer who may benefit from a treatment comprising an anti-cancer therapy, the method comprising detecting in one or more samples from the individual a cluster of differentiation 274 (CD274) gene copy number gain and a high tumor mutational burden (TMB), wherein detection of the CD274 gene copy number gain and the high TMB in the one or more samples identifies the individual as one who may benefit from a treatment comprising an anti-cancer therapy.

Exemplary Embodiment 220: A method of selecting a treatment for an individual having a cancer, the method comprising detecting in one or more samples from the individual a CD274 gene copy number gain and a high TMB, wherein detection of the CD274 gene copy number gain and the high TMB in the one or more samples identifies the individual as one who may benefit from a treatment comprising an anti-cancer therapy.

Exemplary Embodiment 221: A method of identifying one or more treatment options for an individual having a cancer, the method comprising:
(a) detecting in one or more samples from the individual a CD274 gene copy number gain and a high TMB; and
(b) generating a report comprising one or more treatment options identified for the individual based, at least in part, on detection of the CD274 gene copy number gain and the high TMB in the one or more samples, wherein the one or more treatment options comprise an anti-cancer therapy.

Exemplary Embodiment 222: A method of identifying one or more treatment options for an individual having a cancer, the method comprising:
(a) acquiring knowledge of a CD274 gene copy number gain and a high TMB in one or more samples from the individual; and
(b) generating a report comprising one or more treatment options identified for the individual based, at least in part, on said knowledge, wherein the one or more treatment options comprise an anti-cancer therapy.

Exemplary Embodiment 223: A method of selecting a treatment for an individual having a cancer, comprising acquiring knowledge of a CD274 gene copy number gain and a high TMB in one or more samples from the individual, wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

Exemplary Embodiment 224: A method of predicting survival of an individual having a cancer, comprising acquiring knowledge of a CD274 gene copy number gain and a high TMB in one or more samples from the individual, wherein responsive to the acquisition of said knowledge, the individual is predicted to have longer survival when treated with a treatment comprising an anti-cancer therapy, as compared to survival of an individual whose cancer does not comprise a CD274 gene copy number gain and/or a high TMB.

Exemplary Embodiment 225: A method of predicting survival of an individual having a cancer treated with a treatment comprising an anti-cancer therapy, the method comprising acquiring knowledge of a CD274 gene copy number gain and a high TMB in one or more samples from the individual, wherein responsive to the acquisition of said knowledge, the individual is predicted to have longer survival when treated with a treatment comprising an anti-cancer therapy, as compared to an individual whose cancer does not exhibit a CD274 gene copy number gain and/or a high TMB.

Exemplary Embodiment 226: A method of treating or delaying progression of a cancer in an individual, comprising:
(a) acquiring knowledge of a CD274 gene copy number gain and a high TMB in one or more samples from an individual having a cancer; and
(b) responsive to said knowledge, administering to the individual an effective amount of a treatment that comprises an anti-cancer therapy.

Exemplary Embodiment 227: A method of treating or delaying progression of a cancer in an individual, comprising administering to an individual having a cancer an effective amount of a treatment that comprises an anti-cancer therapy, wherein the anti-cancer therapy is administered responsive to acquiring knowledge of a CD274 gene copy number gain and a high TMB in one or more samples from the individual.

Exemplary Embodiment 228: A method of monitoring, evaluating or screening an individual having a cancer, comprising acquiring knowledge of a CD274 gene copy number gain and a high TMB in one or more samples from the individual, wherein responsive to the acquisition of said knowledge, the individual is predicted to have an improved response to a treatment comprising an anti-cancer therapy and/or longer survival when treated with a treatment comprising an anti-cancer therapy, as compared to an individual whose cancer does not comprise a CD274 gene copy number gain and/or a high TMB.

Exemplary Embodiment 229: A method for monitoring progression or recurrence of a cancer in an individual, the method comprising:
(a) detecting the presence or absence of a CD274 gene copy number gain and a high TMB in one or more samples obtained from the individual at a first time point;
(b) detecting the presence or absence of a CD274 gene copy number gain and a high TMB in one or more samples obtained from the individual at a second time point after the first time point; and
(c) providing an assessment of cancer progression or cancer recurrence in the individual based, at least in part, on the presence or absence of the CD274 gene copy number gain and the high TMB in the one or more samples obtained from the individual at the first and/or second time points.

Exemplary Embodiment 230: The method of embodiment 229, wherein the presence of the CD274 gene copy number gain and the high TMB in the one or more samples obtained from the individual at the first and/or second time points identifies the individual as having decreased risk of cancer progression or cancer recurrence when treated with a treatment comprising an anti-cancer therapy.

Exemplary Embodiment 231: The method of embodiment 229 or embodiment 230, further comprising selecting a treatment, administering a treatment, adjusting a treatment, adjusting a dose of a treatment, or applying a treatment to the individual based, at least in part, on detecting the presence of the CD274 gene copy number gain and the high TMB in the one or more samples obtained from the individual at the first and/or second time points, wherein the treatment comprises an anti-cancer therapy.

Exemplary Embodiment 232: A method of identifying a candidate treatment for a cancer in an individual in need thereof, comprising:
performing DNA sequencing on one or more samples obtained from the individual to determine a sequencing mutation profile, wherein the sequencing mutation profile identifies the presence of a CD274 gene copy number gain and a high TMB in the one or more samples; and
selecting a treatment for the individual based, at least in part, on the sequencing mutation profile, wherein the treatment comprises an anti-cancer therapy.

Exemplary Embodiment 233: The method of embodiment 232, wherein the presence of the CD274 gene copy number gain and the high TMB in the one or more samples identifies the individual as one who may benefit from a treatment comprising an anti-cancer therapy.

Exemplary Embodiment 234: The method of embodiment 232 or embodiment 233, wherein the presence of the CD274 gene copy number gain and the high TMB in the one or more samples predicts the individual to have longer survival when treated with a treatment comprising an anti-cancer therapy, as compared to survival of an individual whose cancer does not comprise a CD274 gene copy number gain and a high TMB.

Exemplary Embodiment 235: A method of treating or delaying progression of a cancer in an individual, comprising:
(a) detecting in one or more samples from an individual having a cancer a CD274 gene copy number gain and a high TMB; and
(b) administering to the individual an effective amount of a treatment that comprises an anti-cancer therapy.

Exemplary Embodiment 236: The method of any one of embodiments 219-235, wherein the anti-cancer therapy is any anti-cancer therapy or agent known in the art or provided herein.

Exemplary Embodiment 237: The method of any one of embodiments 219-236, wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 238: A method of identifying an individual having a cancer who may benefit from a treatment comprising an immunotherapy, the method comprising detecting in one or more samples from the individual a cluster of differentiation 274 (CD274) gene copy number gain and a high tumor mutational burden (TMB), wherein detection of the CD274 gene copy number gain and the high TMB in the one or more samples identifies the individual as one who may not benefit from a treatment comprising an anti-cancer therapy and may benefit from an immunotherapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 239: A method of selecting a treatment for an individual having a cancer, the method comprising detecting in one or more samples from the individual a CD274 gene copy number gain and a high TMB, wherein detection of the CD274 gene copy number gain and the high TMB in the one or more samples identifies the individual as one who may not benefit from a treatment comprising an anti-cancer therapy and may benefit from an immunotherapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 240: A method of selecting a treatment for an individual having a cancer, comprising acquiring knowledge of a CD274 gene copy number gain and a high TMB in one or more samples from the individual, wherein responsive to the acquisition of said knowledge the individual is identified as unlikely to respond to a treatment that comprises an anti-cancer therapy and likely to respond to an immunotherapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 241: A method of predicting survival of an individual having a cancer, comprising acquiring knowledge of a CD274 gene copy number gain and a high TMB in one or more samples from the individual, wherein responsive to the acquisition of said knowledge, the individual is predicted to have longer survival when treated with a treatment comprising an immunotherapy, as compared to survival of a corresponding individual treated with an anti-cancer therapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 242: A method of predicting survival of an individual having a cancer treated with a treatment comprising an immunotherapy, the method comprising acquiring knowledge of a CD274 gene copy number gain and a high TMB in one or more samples from the individual, wherein responsive to the acquisition of said knowledge, the individual is predicted to have longer survival when treated with a treatment comprising an immunotherapy, as compared to a corresponding individual treated with an anti-cancer therapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 243: A method of monitoring, evaluating or screening an individual having a cancer, comprising acquiring knowledge of a CD274 gene copy number gain and a high TMB in one or more samples from the individual, wherein responsive to the acquisition of said knowledge, the individual is predicted to have an improved response to a treatment comprising an immunotherapy and/or longer survival when treated with a treatment comprising an immunotherapy, as compared to a corresponding individual treated with an anti-cancer therapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 244: A method of identifying a candidate treatment for a cancer in an individual in need thereof, comprising:
performing DNA sequencing on one or more samples obtained from the individual to determine a sequencing mutation profile, wherein the sequencing mutation profile identifies the presence of a CD274 gene copy number gain and a high TMB in the one or more samples; and
selecting a treatment for the individual based, at least in part, on the sequencing mutation profile.

Exemplary Embodiment 245: The method of embodiment 244, wherein the presence of the CD274 gene copy number gain and the high TMB in the one or more samples identifies the individual as one who may benefit from a treatment comprising an immunotherapy.

Exemplary Embodiment 246: The method of embodiment 244 or embodiment 245, wherein the presence of the CD274 gene copy number gain and the high TMB in the one or more samples predicts the individual to have longer survival when treated with a treatment comprising an immunotherapy, as compared to survival of a corresponding individual treated with an anti-cancer therapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 247: A system for identifying an individual having a cancer who may benefit from a treatment comprising an anti-cancer therapy, the system comprising: a memory configured to store one or more program instructions; and one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from one or more samples obtained from an individual having a cancer;
(b) analyze the plurality of sequence reads for the presence of a CD274 gene copy number gain and a high TMB; and
(c) detect, based on the analyzing, the presence of the CD274 gene copy number gain and high TMB in the one or more samples;
wherein detecting the CD274 gene copy number gain and high TMB identifies the individual as one who may benefit from a treatment comprising an anti-cancer therapy.

Exemplary Embodiment 248: A non-transitory computer readable storage medium comprising one or more programs executable by one or more computer processors for performing a method for identifying an individual having a cancer who may benefit from a treatment comprising an anti-cancer therapy, the method comprising:
(a) obtaining, using the one or more processors, a plurality of sequence reads of one or more nucleic acid molecules, wherein the one or more nucleic acid molecules are derived from one or more samples obtained from an individual having a cancer;
(b) analyzing, using the one or more processors, the plurality of sequence reads for the presence of a CD274 gene copy number gain and a high TMB; and
(c) detecting, using the one or more processors and based on the analyzing, the CD274 gene copy number gain and high TMB in the one or more samples;
   wherein detecting the CD274 gene copy number gain and high TMB identifies the individual as one who may benefit from a treatment comprising an anti-cancer therapy.

Exemplary Embodiment 249: A system for identifying an individual having a cancer who may benefit from a treatment comprising an immunotherapy, the system comprising:
a memory configured to store one or more program instructions; and one or more processors configured to execute the one or more program instructions, the one or more program instructions when executed by the one or more processors are configured to:
(a) obtain a plurality of sequence reads of one or more nucleic acid molecules, wherein the one or more nucleic acid molecules are derived from one or more samples obtained from an individual having a cancer;
(b) analyze the plurality of sequence reads for the presence of a CD274 gene copy number gain and a high TMB; and
(c) detect, based on the analyzing, the presence of the CD274 gene copy number gain and high TMB in the one or more samples;
   wherein detecting the CD274 gene copy number gain and high TMB identifies the individual as one who may not benefit from a treatment comprising an anti-cancer therapy and may benefit from an immunotherapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 250: A non-transitory computer readable storage medium comprising one or more programs executable by one or more computer processors for performing a method for identifying an individual having a cancer who may benefit from a treatment comprising an immunotherapy, the method comprising:
(a) obtaining, using the one or more processors, a plurality of sequence reads of one or more nucleic acid molecules, wherein the one or more nucleic acid molecules are derived from one or more samples obtained from an individual having a cancer;
(b) analyzing, using the one or more processors, the plurality of sequence reads for the presence of a CD274 gene copy number gain and a high TMB; and
(c) detecting, using the one or more processors and based on the analyzing, the CD274 gene copy number gain and high TMB in the one or more samples;
   wherein detecting the CD274 gene copy number gain and high TMB identifies the individual as one who may not benefit from a treatment comprising an anti-cancer therapy and may benefit from an immunotherapy; optionally wherein the anti-cancer therapy is a chemotherapy or chemotherapeutic agent, a targeted therapy, an alkylating agent, an antimetabolite, a natural product, a hormone, a radiation therapy, or a therapy configured to target a defect in a specific cell signaling pathway.

Exemplary Embodiment 251: An anti-cancer therapy for use in a method of treating or delaying progression of cancer in an individual, wherein the method comprises administering the anti-cancer therapy to an individual having a cancer, wherein a CD274 gene copy number gain and a high TMB are detected in one or more samples obtained from the individual.

Exemplary Embodiment 252: An anti-cancer therapy for use in the manufacture of a medicament for treating or delaying progression of cancer in an individual, wherein the medicament is to be administered to an individual having a cancer, wherein a CD274 gene copy number gain and a high TMB are detected in one or more samples obtained from the individual.

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Association of CD274 Copy Number Alterations with Response to Immune Checkpoint Inhibitor Therapy

This Example describes a retrospective clinical study evaluating the association of CD274 copy number and tumor mutational burden (TMB) with responses to immune checkpoint inhibitor (ICI) therapy.

### Materials and Methods

### Patient Cohort

A U.S.-based de-identified clinico-genomic database (CGDB) was used for the study. The de-identified data originated from approximately 280 cancer clinics (˜800 sites of care). Retrospective longitudinal clinical data were derived from electronic health record data, including patient-level structured and unstructured data, curated via technology-enabled abstraction, and linked to genomic data derived from comprehensive genomic profiling (CGP) tests in the CGDB by de-identified, deterministic matching as previously described (Singal, G. et al., (2019), JAMA 21(14): 1391-1399).

The study included 621 patients that satisfied the following cohort-inclusion criteria: (1) a chart-confirmed diagnosis of non-squamous non-small cell lung cancer (NSCLC); (2) had at least two documented clinical visits; (3) underwent CGP testing on a pathologist-confirmed non-squamous NSCLC tumor specimen on or after the date of chart-confirmed initial diagnosis of non-squamous NSCLC, on a sample collected no earlier than 30 days before the diagnosis date; (4) were wild type for any oncogenic EGFR and ALK genomic alteration as determined by CGP tests; and (5) were treated with second line ICI monotherapy, e.g., atezolizumab, durvalumab, nivolumab or pembrolizumab. Patients who had already received any form of ICI in the first line setting were excluded.

### Comprehensive Genomic Profiling

Clinical cases of non-squamous NSCLC (as diagnosed by the treating physician and confirmed on hematoxylin and eosin-stained slides) underwent CGP testing using previously described assays (see, e.g., Frampton, G. M., et al. (2013), Nat. Biotechnol. 31(11):1023-31). All samples submitted for sequencing featured a minimum of 20% tumor cell nuclear area and yielded a minimum of 50 ng of extracted DNA. CGP was performed on hybridization-captured, adapter-ligation based libraries, to identify genomic alterations (base substitutions, small insertions/deletions, copy number alterations and rearrangements) in greater than 300 cancer-associated genes, tumor mutational burden (TMB; see, e.g., Chalmers, Z. R., et al. (2017), Genome Med. 9(1):34), and microsatellite instability status (MSI; see, e.g., Trabucco, S. E., et al. (2019), J. Mol. Diagnostics 21(6): 1053-1066).

### CD274 Copy Number Calling

CD274 copy number alterations were detected using a comparative genomic hybridization-like method applied to next-generation sequencing data (see, e.g., Frampton, G. M., et al. (2013), Nat. Biotechnol. 31(11):1023-31; Sun, J. X., et al. (2018), PLoS Pathog. 14(2):e1005965). In the laboratory, each specimen was analyzed alongside a process-matched normal control (an internally validated mixture of 10 heterozygous diploid samples from the HapMap project), with custom algorithms to normalize the sequence coverage distribution across captured DNA regions. Log-ratios of normalized coverage data for exonic, intronic, and SNP targets accounting for stromal admixture, as well as genome-wide SNP frequencies, were used to generate the profiles. Using circular binary segmentation, custom algorithms further clustered groups of targets and SNP frequencies to define upper and lower bounds of genomic segments. Empirical Bayesian algorithms employed a distribution of parameters including purity and base ploidy, and probability matrices were derived using different statistical sampling methodologies to fit these data. Specimen level ploidy was estimated as previously described (see, e.g., Sun, J. X., et al. (2018), PLoS Pathog. 14(2): e1005965). Computational models were reviewed by expert analysts for each sample (see, e.g., Frampton, G. M., et al. (2013), Nat. Biotechnol. 31(11):1023-31).

### PD-L1 Expression

PD-L1 Immunohistochemistry (IHC) testing was performed for a subset of specimens in the CGDB cohort. DAKO PD-L1 IHC 22C3 pharmDx's tumor proportion scoring (TPS) method was used to score the cases (DAKO PD-L1 IHC 22C3 pharmDx Interpretation Manual—NSCLC. Accessible at: www.agilent.com/cs/library/usermanuals/public/29158_pd-11-ihc-22C3-pharmdx-nsclc-interpretation-manual.pdf). TPS was calculated as the number of PD-L1 positive tumor cells divided by the total number of PD-L1 positive and PD-L1 negative tumor cells.

### Outcomes and Statistical Analyses

The primary clinical end point of this study was overall survival (OS) from start of second line ICI monotherapy until death or loss of follow-up. To account for delayed entry into the real-world clinico-genomic cohort, risk set adjustment was performed to adjust for left truncation bias. The Kaplan-Meier method along with the log-rank test were used to estimate differences between outcome estimates. Categorical variables were compared using the two-sided Fisher's exact test, while the two-sided Wilcoxon rank sum test was used to compare continuous variables. All analyses were performed using the R software (Ihaka, R., and Gentleman, R. (1996), Journal of Computational and Graphical Statistics 5(3): 299-314) version 4.0.3.

### Results

### Patient Characteristics

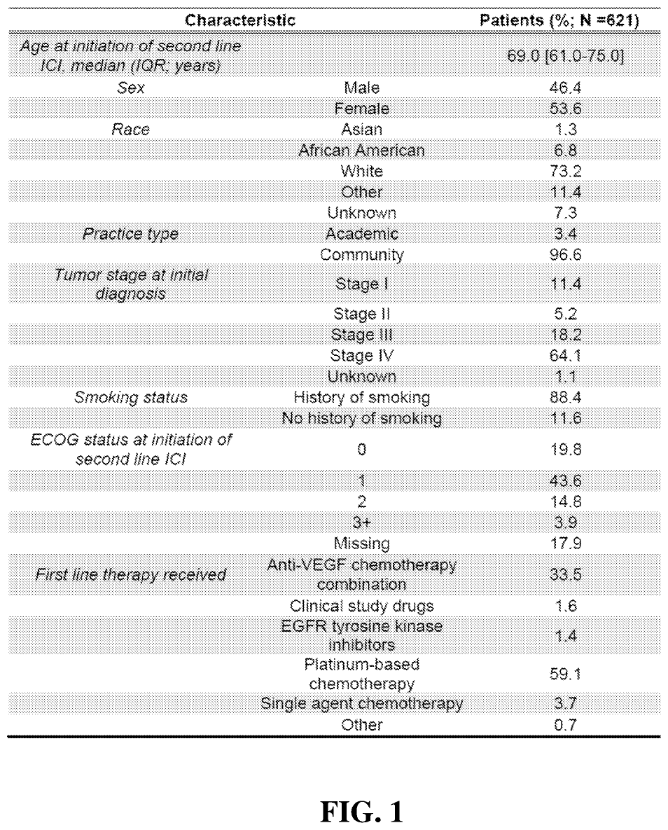

Overall, 621 EGFR- and ALK-wild-type non-squamous NSCLC patients treated with second line ICI monotherapy that fit the pre-defined inclusion criteria were identified. The median (inter-quartile range) follow-up time was 10.9 (3.7-23.4) months, and as of the CGDB data cut-off date, 73.3% had died. As shown in FIG. 1, the majority of the patients were female (53.6%), had a self-reported race as White (73.2%), and were stage IV at the time of initial diagnosis (64.1%). Moreover, 18.7% had an Eastern Cooperative Oncology Group (ECOG) score over 2 at initiation of second line ICI monotherapy, and 88.4% had a history of smoking. Among the 621 patients, 59.1% and 33.5% of the patients had received either platinum-based chemotherapy or anti-VEGF combination therapy, respectively, in the first-line setting (FIG. 1).

### Biomarker Characteristics

Out of the 621 patients, 20% (124/621) were assessed for PD-L1 IHC expression. Among the patients assessed for PD-L1 expression, 41.1% had a PD-L1 TPS score greater than or equal to 50%, 30.6% had a PD-L1 TPS score between 1% and 49%, and 28.2% had a PD-L1 TPS score of less than 1%. The median TMB of the CGDB cohort (N=621) was 8.8 (inter-quartile range [IQR]=3.5-14.8) mutations per Mb (mut/Mb), while 45.4% of patients had a TMB greater than or equal to 10 mut/Mb. An MSI-high status was observed for 0.5% of the cohort.

Association of CD274 Copy Number with Response to ICI Blockade

Across the overall cohort, 1.4% of patients had a CD274 copy number (CN) greater than or equal to specimen ploidy +4, 2.4% had a CD274 CN greater than or equal to specimen ploidy +3, 4.7% had a CD274 CN greater than or equal to specimen ploidy +2, and 15.0% had a CD274 CN greater than or equal to specimen ploidy +1. In addition, 36.9% of patients had a CD274 CN equal to specimen ploidy. Among patients with a CD274 loss, 48.1% had a CD274 copy number lesser than or equal to specimen ploidy −1, 11.8% had a CD274 CN lesser than or equal to specimen ploidy −2, and 1.1% had a CD274 CN lesser than or equal to specimen ploidy −3.

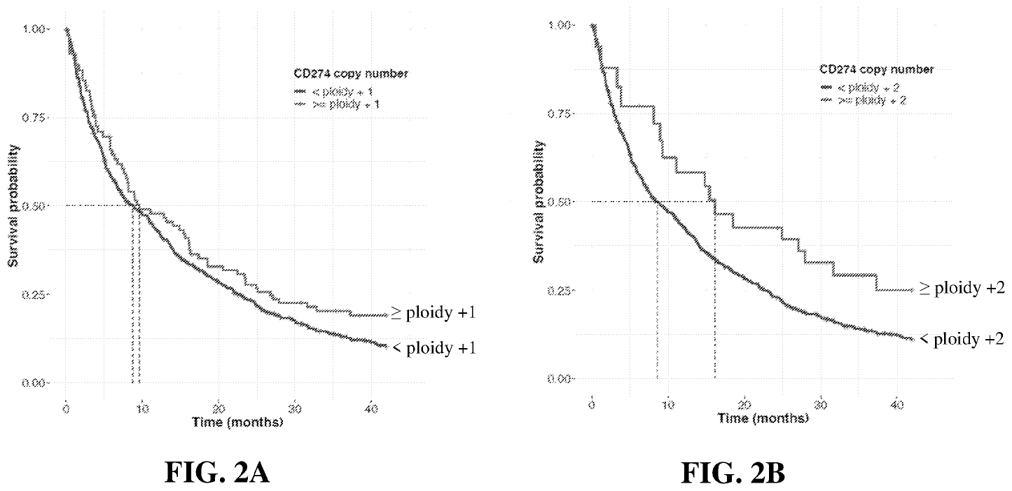

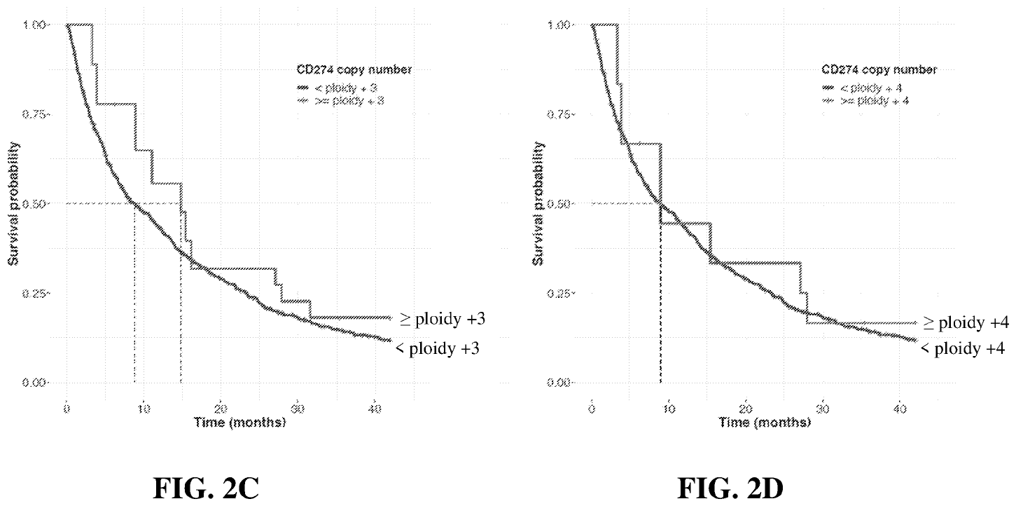

To examine the association of CD274 CN and ICI blockade responses, the overall survival (OS) of patients from the start of second-line ICI monotherapy was assessed at various CD274 CN thresholds. When assessing the effect of CD274 CN gain as a positive predictor of OS with ICI monotherapy, it was observed that at a CD274 CN threshold of greater than or equal to specimen ploidy +1, the gain group had a higher median OS (mOS) when compared to the rest of the patients (FIG. 2A; gain group (N=93) mOS=9.6 (95% CI=7.6-16.2) months; rest of patients (N=538) mOS=8.8 (95% CI=6.9-11.2) months; p=0.09). At a CD274 CN threshold of greater than or equal to specimen ploidy +2, the gain group had significantly higher mOS when compared to the rest of the patients (FIG. 2B; gain group (N=29) mOS=16.1 (95% CI=8.9-37.3) months; rest of patients (N=592) mOS=8.6 (95% CI=7.1-10.9) months; p=0.05). At a CD274 CN threshold of greater than or equal to specimen ploidy +3, the gain group had higher mOS when compared to the rest of the patients (FIG. 2C; gain group (N=15) mOS=14.8 (95% CI=8.9-NA) months; rest of patients (N=606) mOS=8.8 (95% CI=7.3-11) months; p=0.5). Finally, at a CD274 CN threshold of greater than or equal to specimen ploidy +4, the gain group had a mOS comparable to the rest of the patients (FIG. 2D; gain group (N=9) mOS=8.94 (95% CI=3.8-NA); rest of patients (N=612) mOS=8.9 (95% CI=7.3-11.2) months; p=0.7). As the CD274 copy number threshold was increased from at least specimen ploidy +1 to at least specimen ploidy +4, the 1-year OS rate among the patients with CD274 CN gains was observed to be 61.1%, 73.3%, 75% and 66.7%, respectively.

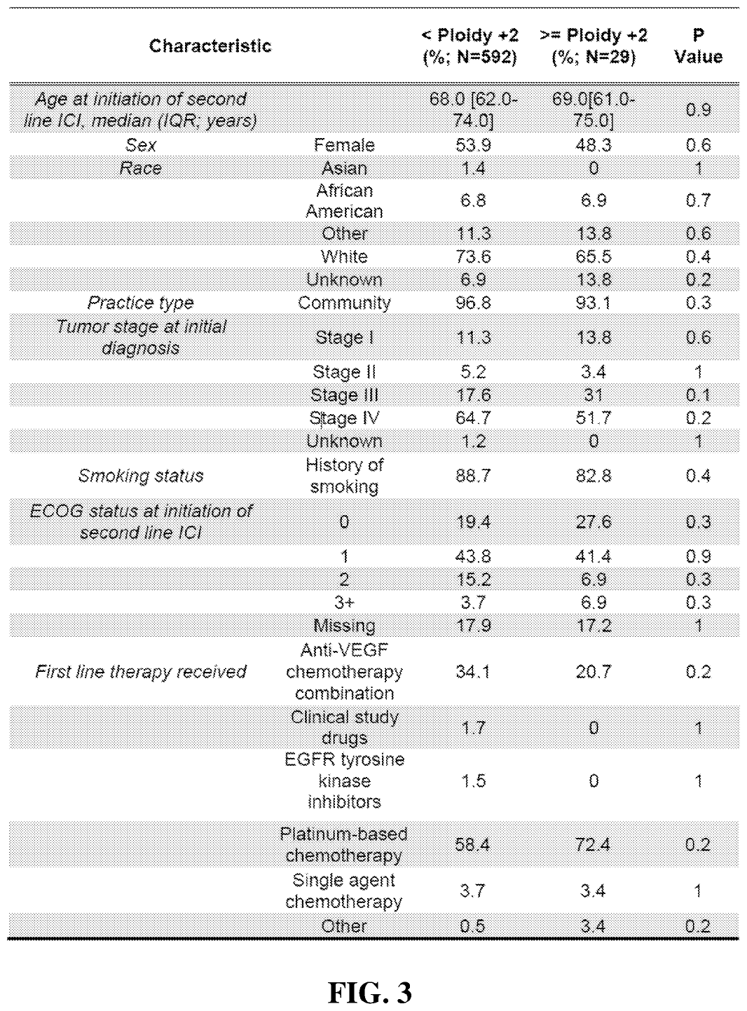

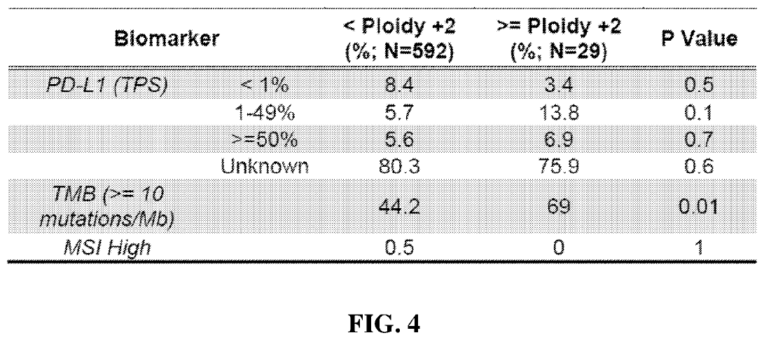

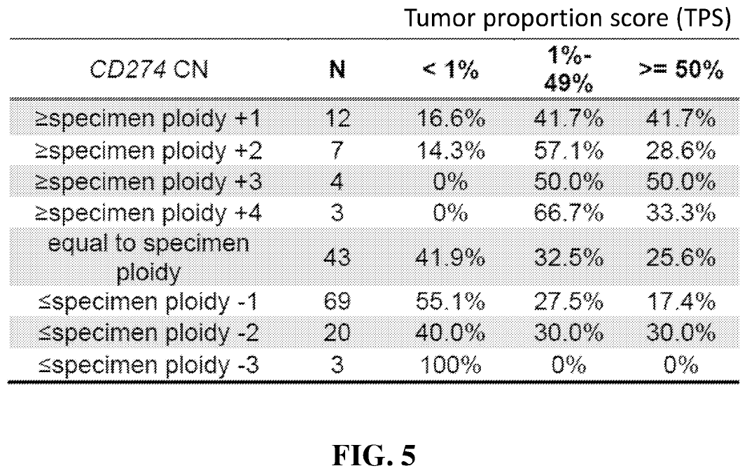

Given the significantly higher survival of patients at a CD274 CN threshold of greater than or equal to specimen ploidy +2, the patient cohort was specifically examined using the ploidy +2 cut-off. No significant differences in the demographics and clinical characteristics of the gain group at a CD274 CN threshold greater than or equal to specimen ploidy +2 were observed as compared to the rest of the patients (FIG. 3). However, among the well-studied ICI biomarkers of PD-L1, TMB and MSI, TMB-High status (at a threshold of 10 mutations/Mb) was significantly enriched in the gain group as compared to the rest of the patients (FIG. 4). For the subset of patients with available PD-L1 protein expression data, CD274 CN changes were overall correlated with PD-L1 protein expression (FIG. 5), but CD274 CN changes were not entirely concordant with PD-L1 protein expression (i.e., cases with CD274 CN gain with no PD-L1 protein expression, as well as cases with CD274 CN loss with PD-L1 protein expression, existed in this cohort).

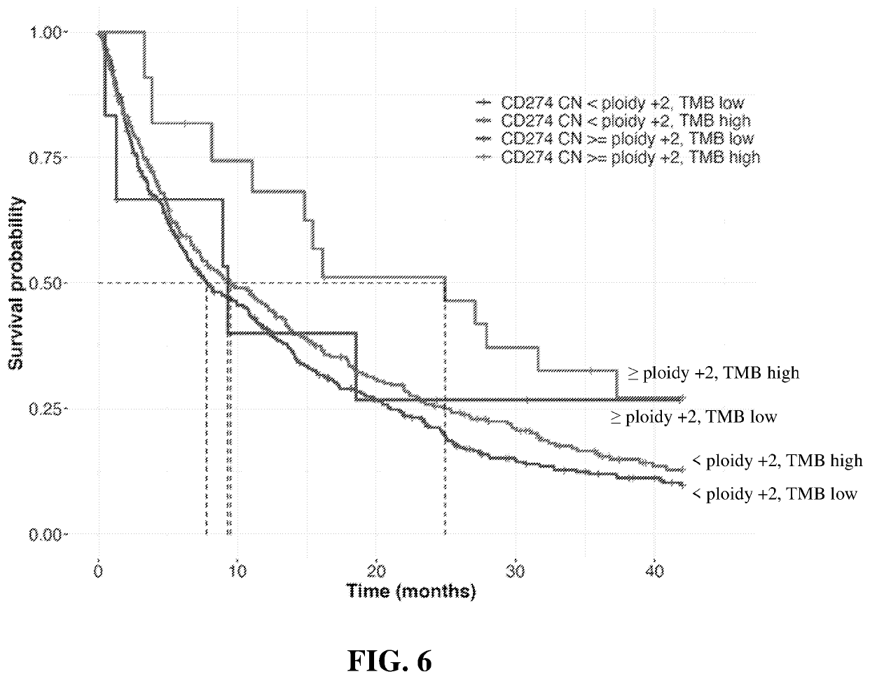

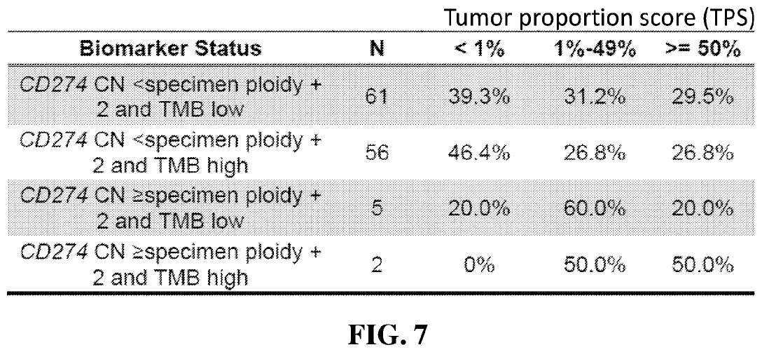

At a CD274 CN gain threshold of 2, when the OS from the start of second-line ICI monotherapy was stratified by TMB-high status, a synergistic pattern emerged. The mOS of patients with CD274 CN less than specimen ploidy +2 and TMB-low status (N=330) was the lowest at 7.7 (CI=6.3-10.9) months. The mOS of patients with CD274 CN less than specimen ploidy +2 and TMB-high status (N=262) was comparable to that of patients with CD274 CN greater than or equal to specimen ploidy +2 and TMB-low status (N=9), at 9.5 (CI=7.1-13.2) months and 9.3 (CI=1.3-NA) months, respectively. Unexpectedly, the mOS of patients with CD274 CN greater than or equal to ploidy +2 and TMB-high status (N=20) was the highest at 24.9 months (CI=11.1-NA; p=0.04; FIG. 6). Moreover, the increase in mOS between the TMB-high and TMB-low groups was much higher in patients with CD274 CN greater than or equal to specimen ploidy +2 (15.6 months) compared to that for patients with CD274 CN lower than specimen ploidy +2 (1.8 months). FIG. 7 shows the distribution of PD-L1 IHC status across TMB (at a threshold of 10 mut/Mb) and CD274 CN (at a CN gain threshold of 2) groups.

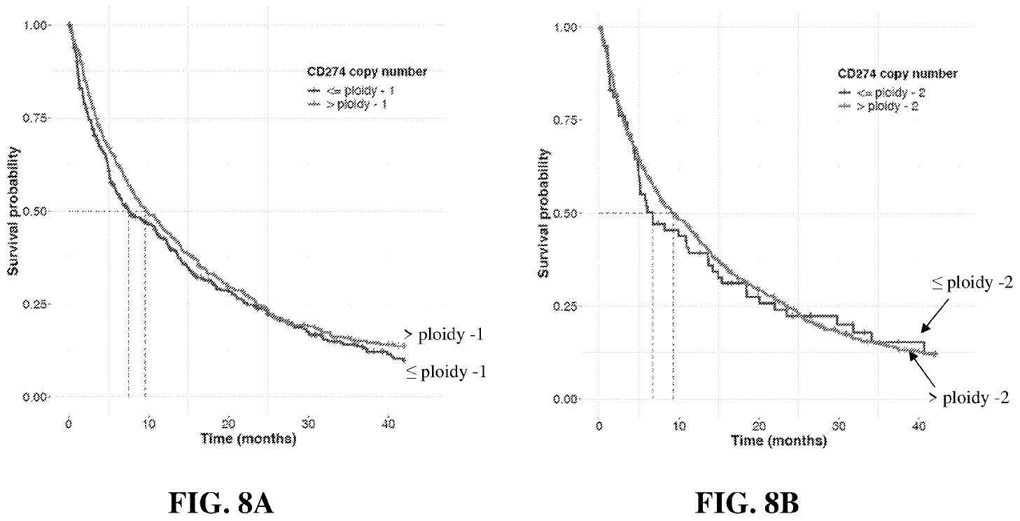

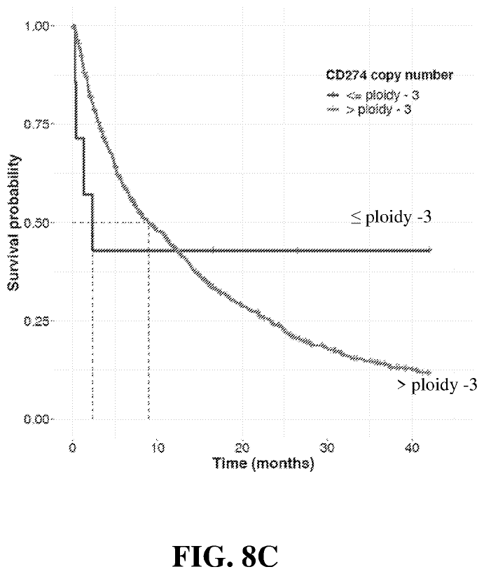

It was also observed that CD274 loss, defined as a CD274 CN lesser than or equal to specimen ploidy −1 (N=299), trended towards lower mOS (mOS=7.5 [CI=5.9-11.3]months), when compared to the rest of the cohort (N=322, mOS=9.6 [CI=7.9-12.8]months; p=0.3; FIG. 8A). When the CD274 loss threshold was lowered to a CD274 CN lesser than or equal to specimen ploidy −2, the mOS for the loss group (N=73) was 6.7 (CI=4.9-14.2) months, whereas the rest of the cohort (N=548) had a mOS of 9.3 (CI=7.5-11.5) months (p=0.8; FIG. 8B). Finally, at a CD274 CN threshold of lesser than or equal to specimen ploidy −3, the mOS for the loss group (N=7) dropped further to 2.3 (CI=0.4-NA) months, whereas the rest of the cohort (N=614) had a mOS of 8.9 (CI=7.4-11.2) months (p=0.6; FIG. 8C).

### CONCLUSIONS

CD274 copy number changes can be used as positive or negative predictors of response to ICI therapy. Moreover, CD274 copy number gains at a copy number of specimen ploidy +2 (which corresponds to at least 4 copies of CD274) in combination with TMB-high status can be used to predict response to ICI therapy better than either of these biomarkers alone.

What is claimed is:

1. A method of treating or delaying progression of a cancer in an individual, comprising:
(a) detecting in one or more samples from an individual having a cancer a cluster of differentiation 274 (CD274) gene copy number gain and a high tumor mutational burden (TMB); and
(b) administering to the individual an effective amount of a treatment that comprises an immunotherapy.

2. A method of identifying one or more treatment options for an individual having a cancer, the method comprising:
(a) detecting in one or more samples from the individual a CD274 gene copy number gain and a high TMB; and
(b) generating a report comprising one or more treatment options identified for the individual based, at least in part, on detection of the CD274 gene copy number gain and the high TMB in the one or more samples, wherein the one or more treatment options comprise an immunotherapy.

3. A method of identifying a candidate treatment for a cancer in an individual in need thereof, comprising:
performing DNA sequencing on one or more samples obtained from the individual to determine a sequencing mutation profile, wherein the sequencing mutation profile identifies the presence of a CD274 gene copy number gain and a high TMB in the one or more samples; and
selecting a treatment for the individual based, at least in part, on the sequencing mutation profile, wherein the treatment comprises an immunotherapy.

4. The method of claim 1, wherein the CD274 gene copy number gain in a sample from the individual comprises a CD274 gene copy number of any of at least +1, at least +2, at least +3, at least +4, at least +5, at least +6, at least +7, at least +8, or more, as compared to the ploidy of the sample from the individual.

5. The method of claim 4, wherein the CD274 gene copy number gain in a sample from the individual comprises a CD274 gene copy number of at least +2, as compared to the ploidy of the sample from the individual.

6. The method of claim 4, wherein the ploidy of the sample from the individual is monoploid, diploid, triploid, or tetraploid.

7. The method of claim 1, wherein:
the CD274 gene copy number gain in a sample from the individual comprises a CD274 gene copy number of any of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is monoploid;
the CD274 gene copy number gain in a sample from the individual comprises a CD274 gene copy number of any of at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is diploid;
the CD274 gene copy number gain in a sample from the individual comprises a CD274 gene copy number of any of at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is triploid; or
the CD274 gene copy number gain in a sample from the individual comprises a CD274 gene copy number of any of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, or more, wherein the ploidy of the sample from the individual is tetraploid.

8. The method of claim 1, wherein the CD274 gene copy number gain is detected by fluorescence in situ hybridization (FISH), comprehensive genomic profiling (CGP), comparative genomic hybridization (CGH), sequencing, or any combination thereof.

9. The method of claim 1, wherein detecting a CD274 gene copy number gain comprises:
(a) providing a plurality of nucleic acid molecules obtained from a sample from an individual having a cancer, wherein the plurality of nucleic acid molecules comprises nucleic acid molecules corresponding to a CD274 gene, or a portion thereof;
(b) optionally, ligating one or more adapters onto one or more nucleic acid molecules from the plurality of nucleic acid molecules;
(c) optionally, amplifying the one or more ligated nucleic acid molecules from the plurality of nucleic acid molecules;
(d) optionally, capturing amplified nucleic acid molecules from the amplified nucleic acid molecules;
(e) sequencing, by a sequencer, the captured nucleic acid molecules to obtain a plurality of sequence reads that represent the captured nucleic acid molecules, wherein one or more sequence reads of the plurality of sequence reads correspond to a CD274 gene, or a portion thereof;
(f) analyzing the plurality of sequence reads for the presence or absence of a CD274 gene copy number gain; and
(g) based on the analyzing step, detecting the presence or absence of a CD274 gene copy number gain in the sample.

10. The method of claim 1, wherein detecting a CD274 gene copy number gain comprises:
(a) providing a sample from an individual having a cancer, wherein the sample comprises a plurality of nucleic acid molecules;
(b) preparing a nucleic acid sequencing library from the plurality of nucleic acid molecules in the sample;
(c) amplifying said library;
(d) selectively enriching for one or more nucleic acid molecules comprising nucleotide sequences corresponding to a CD274 gene or a portion thereof in said library to produce an enriched sample;
(e) sequencing the enriched sample, thereby producing a plurality of sequence reads;
(f) analyzing the plurality of sequence reads for the presence or absence of a CD274 gene copy number gain; and
(g) detecting, based on the analyzing step, the presence or absence of a CD274 gene copy number gain in the sample from the individual.

11. The method of claim 9, wherein the analyzing step comprises:
(a) generating a copy number model based on the plurality of sequence reads; and
(b) determining a CD274 gene copy number based on the copy number model.

12. The method of claim 9, wherein the analyzing step comprises:
determining coverage ratio data, allele fraction data, and segmentation data for one or more gene loci within one or more subgenomic intervals of the plurality of sequence reads, wherein the one or more gene loci comprise CD274;
identifying a plurality of segments based on the segmentation data;
determining copy numbers for the plurality of segments based on the coverage ratio data, the allele fraction data, the segmentation data, and a copy number model; and
detecting the presence or absence of a CD274 copy number gain based on a copy number of a segment of the plurality of segments corresponding to CD274, wherein a CD274 copy number gain is detected when the copy number for the segment of the plurality of segments corresponding to CD274 is greater than ploidy of the sample.

13. The method of claim 1, wherein the high TMB comprises a TMB of at least about 5 mutations/Megabase (mut/Mb) or at least about 10 mut/Mb.

14. The method of claim 1, further comprising:
(a) assessing microsatellite instability status of the cancer in a sample from the individual;
(b) assessing expression of PD-L1 protein in a sample from the individual; and/or
(c) acquiring knowledge of or detecting in a sample from the individual a base substitution, a short insertion/deletion (indel), a copy number alteration, or a genomic rearrangement in one or more genes.

15. The method of claim 1, wherein the individual is a human.

16. The method of claim 1, wherein the cancer is:
(a) a solid tumor or a hematologic malignancy; or
(b) a B cell cancer, multiple myeloma, melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

17. The method of claim 1, wherein the cancer is a non-small cell lung cancer or a non-squamous non-small cell lung cancer.

18. The method of claim 17, wherein the CD274 gene copy number gain in a sample from the individual comprises:
(a) a CD274 gene copy number of at least +2, as compared to the ploidy of the sample from the individual; or
(b) a CD274 gene copy number of at least 4, wherein the ploidy of the sample from the individual is diploid;
and wherein the cancer comprises a TMB of at least about 10 mut/Mb.

19. The method of claim 1, wherein the cancer does not comprise any oncogenic alterations in an EGFR and/or an ALK gene, or is wild type for oncogenic alternations in an EGFR and/or an ALK gene.

20. The method of claim 1, wherein the immunotherapy is an immune checkpoint inhibitor, a cancer vaccine, a cell-based therapy, a T cell receptor (TCR)-based therapy, an adjuvant immunotherapy, a cytokine immunotherapy, or an oncolytic virus therapy.


SEQUENCE LISTING (2 sequences)

SEQ ID NO: 1 - DNA, 3634 bp, Homo sapiens
  source 1..3634: mol_type = unassigned DNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET